(19) United States
(12) Patent Application Publication
(10) Pub. No.: US 2004/0146872 A1
(43) Pub. Date: Jul. 29, 2004

(54) Fat regulated genes, uses thereof and compounds for modulating same

(76) Inventors: Michael David Winther, Vancouver (CA); Leah Christine Knickle, Hammonds Plains (CA); Martin Haardt, Edmonton (CH); Stephen John Allen, Coldbrooke (CA); Andre Ponton, St. Hubert (CA); Roberto Justo De Antueno, Coldbrook (CA); D. Kenneth Jenkins, Coldbrook (CA); Solomon O. Nwaka, Halifax (CA); Y. Paul Goldberg, Vancouver (CA)

Correspondence Address: David J Heller, Ridout & Maybee, Suite 2400, One Queen Street East, Toronto Ontario, M5C 3B1, CA

(21) Appl. No.: 10/416,914

(22) PCT Filed: Nov. 19, 2001
(86) PCT No.: PCT/CA01/01632

(51) Int. Cl. C12Q 1/68; A01K 67/027; C07H 21/04

(52) U.S. Cl. 435/6; 435/69.1; 435/320.1; 435/325; 530/350; 530/388.1; 536/23.2; 800/14

(57) ABSTRACT

The present invention relates to methods for identifying genes regulated by fat and their subsequent use in determining compositions for use in the treatment of disease, by utilizing the identified genes and their proteins. The identified compositions regulate the expression of the fat regulated genes or modulate the activity of their protein products. The nucleotide and amino acid sequences for METP, a novel mitochondrial carrier protein and FTF1, a fat responsive transcription factor, are taught. The nucleotide sequence for A1BG, a plasma glycoprotein is taught. The nucleotide and amino add sequences for human GPAT and rat GLOL are taught. Control sequences for human METP, GLOL, FTF1, SCD, GPAT and A1BG are taught. Polyunsaturated fatty acid regulation functions are taught for SCD1, SCD2, SCD, G6PD, GPAT, FAS, COMT, Apo A-1, INSIG1, FTF1, GLOL, A1GB, Spot-14, delta-3, delta-2-enoyl-CoA isomerase, and METP

### FIELD OF THE INVENTION

This invention relates to methods for the identification of (a) compositions that modulate the activities of proteins whose genes are fat regulated, (b) compositions that modulate the activity of fat regulated genes and (c) compositions that effectively regulate the expression of the fat regulated genes, and to compositions so identified.

### BACKGROUND OF THE INVENTION

Deficiencies in polyunsaturated fatty acids (PUFAs) have been associated with a number of diseases such as eczema, cardiovascular disorders, inflammation, psychiatric disorders, cancer, cystic fibrosis, pre-menstrual syndrome and diabetes (Horrobin D. F., 1990, Pathophysiology and Roles in Clinical Medicine, Wiley-Liss, NY and Mazza G. and Domah B. D.(eds.), 2000, Herbs, Botanicals and Teas, Technomic Publishers, Lancaster, Pa.). Supplementing diet with PUFAs have been attempted as a treatment for a number of these conditions. The level of success for such applications has varied considerably.

It has been observed that PUFAs can alleviate and correct some of the symptoms of diabetic neuropathy (Dines et al., 1993, Diabetologia, 36: 1132-1138 and Cotter et al., 1995, Diabetic Neuropathy: New Concepts and Insights, Elsevier Science B. V., Amsterdam, pp. 115-120). Researchers have speculated that the production or modulation of the cyclooxygenase and lipoxygenase metabolites of the n-3 and n-6 fatty acid families is responsible for some of these beneficial effects. Cameron et al. (1993, Br. J. Pharmacol., 109: 972-979) and Dines et al. (1996, Prostaglandins Leukotr. Essent. Fatty, Acids, 55: 159-165) both hypothesized that by increasing the levels of vasodilator prostaglandins or by reducing vasoconstrictor and platelet aggregatory prostaglandins, symptoms of diabetic neuropathy may be alleviated. Only limited success in developing fatty acids as drugs has been achieved. In part this is due to the complex metabolism of orally administered fatty acids which do not behave like many of the xenobiotic drugs.

### SUMMARY OF THE INVENTION

The present invention teaches an isolated polynucleotide segment, comprising a polynucleotide sequence which is selected from the group consisting of: (a) a sequence comprising SEQ ID NO:1; (b) a sequence comprising SEQ ID NO:3; (c) a sequence comprising SEQ ID NO:6; (d) a sequence comprising SEQ ID NO:11; (e) a sequence comprising SEQ ID NO:13; (f) a sequence comprising SEQ ID NO:16; (g) a sequence comprising SEQ ID NO:18; (h) a sequence comprising SEQ ID NO:24; (i) a sequence which is at least 80% homologous with a sequence of any of (a) to (h); (j) a sequence which is at least 90% homologous with a sequence of any of (a) to (h); (k) a sequence which is at least 95% homologous with a sequence of any of (a) to (h); (i) a sequence which is at least 98% homologous with a sequence of any of (a) to (h); (m) a sequence which is at least 99% homologous with a sequence of any of (a) to (h); (n) a sequence which hybridizes to any of (a) to (m) under stringent conditions; and, (o) a sequence with is a functional derivative of any of (a) to (m). The invention includes an isolated polynucleotide segment, comprising a polynucleotide sequence which retains substantially the same biological function or activity as the polynucleotide of the invention. In an embodiment, the isolated polynucleotide segment is cDNA.

The invention teaches an isolated polypeptide segment comprising an isolated polypeptide selected from the group consisting of: (a) a sequence comprising SEQ ID NO:2; (b) a sequence comprising SEQ ID NO:4; (c) a sequence comprising SEQ ID NO:7; (d) a sequence comprising SEQ ID NO:12; (e) a sequence comprising SEQ ID NO:14; (f) a sequence comprising SEQ ID NO:17; (g) a sequence comprising SEQ ID NO: 25; (h) a sequence which is at least 80% homologous with a sequence of any of (a) to (g); (i) a sequence which is at least 90% homologous with a sequence of any of (a) to (g); (j) a sequence which is at least 95% homologous with a sequence of any of (a) to (g); (k) a sequence which is at least 98% homologous with a sequence of any of (a) to (g); and, (l) a sequence which is at least 99% homologous with a sequence of any of (a) to (g).

The invention includes a host cell comprising a polypeptide segment of the invention in a host cell which is heterologous to the segment. The invention also teaches a process for producing a polypeptide of a segment of the invention comprising the step of culturing the host cell of the invention under conditions sufficient for the production of the polypeptide. The invention includes a polypeptide sequence which retains substantially the same biological function or activity as the polypeptide in a segment of the invention.

The invention also teaches a method for identifying a compound which inhibits or promotes the activity of a polypeptide segment of the invention, comprising the steps of: (a) selecting a control animal having the segment and a test animal having the segment; (b) treating the test animal using a compound; (c) determining the relative quantity or relative activity of an expression product of the segment or of the segment, as between the control animal and the test animal. The animals may be mammals. The mammals may be rats.

In another embodiment, the invention teaches a method for identifying a compound which inhibits or promotes the activity of a polypeptide segment of the invention, comprising the steps of: (a) selecting a host cell of the invention; (b) cloning the host cell and separating the clones into a test group and a control group; (c) treating the test group using a compound; and (d) determining the relative quantity or relative activity of an expression product of the segment or of the segment, as between the test group and the control group.

In another embodiment, the invention includes a method for identifying a compound which inhibits or promotes the activity of a polypeptide segment of the invention, comprising the steps of: (a)selecting a test group having a host cell of the invention, a part thereof or an isolated polypeptide thereof and a control group; (b) treating the test group using a compound; and (c) determining the relative quantity or relative activity of a product of the segment or of the segment, as between the test group and the control group.

In another embodiment, the invention teaches an isolated polynucleotide segment, comprising a polynucleotide sequence which is selected from the group consisting of: (a) a sequence comprising SEQ ID NO:5; (b) a sequence comprising SEQ ID NO:10; (c) a sequence comprising SEQ ID NO:15; (d) a sequence comprising SEQ D NO:20; (e) a sequence comprising SEQ ID NO:21; (f a sequence comprising SEQ ID NO:26; (g) a sequence which is at least 80% homologous with a sequence of any of (a) to (f); (h) a sequence which is at least 90% homologous with a sequence of any of (a) to (f); (i) a sequence which is at least 95% homologous with a sequence of any of (a) to (f); (j) a sequence which is at least 98% homologous with a sequence of any of (a) to (f); (k) a sequence which is at least 99% homologous with a sequence of anyof (a) to (f); and; (l) a sequence which hybridizes to any of (a) to (k) under stringent conditions. The isolated polynucleotide segment may be genomic DNA.

The invention also teaches a vector comprising a polynucleotide segment of the invention. The vector may be heterologous to the segment. The vector may contain or encode a tag. The invention teaches a host cell comprising a polynucleotide segment of the invention in a host cell which is heterologous to the segment.

In other embodiments, the invention includes an isolated polynucleotide fragment selected from the group consisting of: (a) a sequence having at least 15 sequential bases of nucleotides of a segment of the invention; (b) a sequence having at least 30 sequential bases of nucleotides of a segment of the invention; and (c) a sequence having at least 50 sequential bases of nucleotides of a segment of the invention.

The invention teaches a method for identifying a compound which inhibits or promotes the activity of a polynucleotide segment of the invention, comprising the steps of: (a) selecting a control animal having the segment and a test animal having the segment; (b) treating the test animal using a compound; and, (c) determining the relative quantity of an expression product of the segment, as between the control animal and the test animal. The animals may be mammals. The mammals may be rats.

The invention teaches a method for identifying a compound which inhibits or promotes the activity of a polynucleotide segment of the invention, comprising the steps of: (a) selecting a host cell of the invention; (b) cloning the host cell and separating the clones into a test group and a control group; (c) treating the test group using a compound; and (d) determining the relative quantity of an expression product of the segment, as between the test group and the control group.

The invention further teaches a method for identifying a compound which inhibits or promotes the activity of a polynucleotide segment of the invention, comprising the steps of: (a) selecting a test group having a host cell of the invention, a part thereof or an isolated polynucleotide thereof and a control group; (b) treating the test group using a compound; and (c) determining the relative quantity or relative activity of a product of the segment or of the segment, as between the test group and the control group.

In another embodiment, the invention teaches a composition for treating a disorder involving fatty acid regulated genes, the composition comprising a compound which modulates a segment according to the invention and a pharmaceutically acceptable carrier. The invention further teaches the use of a composition of the invention for treating a disorder involving fatty acid regulated genes.

The invention also teaches a method for diagnosing the presence of or a predisposition for a disorder involving fatty acid regulated genes in a subject, the method comprising detecting a germline alteration in a segment of the invention in the subject, comprising comparing the germline sequence of a segment of the invention from a tissue sample from the subject with the germline sequence of a wild-type of the segment, wherein an alteration in the germline sequence of the subject indicates the presence of or a predisposition to the disorder.

The invention teaches a method for diagnosing the presence of or a predisposition for a disorder involving fatty acid regulated genes in a subject, the method comprising comparing the sequence of a polypeptide of the invention from a tissue sample from the subject with the sequence of a wild-type of the polypeptide, wherein an alteration in the sequence of the subject as compared to the wild-type indicates the presence of or a predisposition to the disorder involving genes altered by fatty acids.

The invention teaches a method for identifying a compound which inhibits or promotes the activity of a polynucleotide, wherein the polynucleotide selected from the group consisting of SCD1, SCD2, SCD, G6PD, GPAT, FAS, COMT, ApoA-1, INSIG1, GLOL, Spot-14 and delta-3, delta-2-enoyl-CoA isomerase, comprising the steps of: (a) selecting a control animal having the polynucleotide and a test animal having the polynucleotide; (b) treating the test animal using a compound; and, (c) determining the relative quantity of an expression product of the polynucleotide, as between the control animal and the test animal.

The invention teaches a method for identifying a compound which inhibits or promotes the activity of a polynucleotide, wherein the polynucleotide selected from the group consisting of SCD1, SCD2, SCD, G6PD, GPAT, FAS, COMT, ApoA-1, INSIG1, GLOL, Spot-14 and delta-3, delta-2-enoyl-CoA isomerase, comprising the steps of: (a) selecting a host cell comprising the polynucleotide wherein such host cell is heterologous to the polynucleotide; (b) cloning the host cell and separating the clones into a test group and a control group; (c) treating the test soup using a compound; and (d) determining the relative quantity of an expression product of the polynucleotide, as between the test group and the control group.

The invention teaches a method for identifying a compound which inhibits or promotes the activity of a polynucleotide, wherein the polynucleotide selected from the group consisting of SCD1, SCD2, SCD, G6PD, GPAT, FAS, COMT, ApoA-1, INSIG1, GLOL, Spot-14 and delta-3, delta-2-enoyl-CoA isomerase, comprising the steps of: (a) selecting a test group having a host cell comprising the polynucleotide wherein such host cell is heterologous to the polynucleotide, a part thereof or an isolated polynucleotide thereof and a control group; (b) treating the test group using a compound; and (c) determining the relative quantity or relative activity of a product of the polynucleotide or of the polynucleotide, as between the test group and the control group.

The invention teaches a method for identifying a compound which inhibits or promotes the activity of a polypeptide, wherein the polypeptide selected from the group consisting of SCD1, SCD2, SCD, G6PD, GPAT, FAS, COMT, ApoA-1, INSIG1, GLOL, A1BG, Spot-14 and delta-3, delta-2-enoyl-CoA isomerase, comprising the steps of: (a) selecting a control animal having the polypeptide and a test animal having the polypeptide; (b) treating the test animal using a compound; (c) determining the relative quantity or relative activity of an expression product of the polypeptide or of the polypeptide, as between the control animal and the test animal.

The invention teaches a method for identifying a compound which inhibits or promotes the activity of a polypeptide, wherein the polypeptide selected from the group consisting of SCD1, SCD2, SCD, G6PD, GPAT, FAS, COMT, ApoA-1, INSIG1, GLOL, A1BG, Spot-14 and delta-3, delta-2-enoyl-CoA isomerase, comprising the steps of: (a) selecting a host cell comprising the polypeptide wherein such host cell is heterologous to the polypeptide; (b) cloning the host cell and separating the clones into a test group and a control group; (c) treating the test group using a compound; and (d) determining the relative quantity or relative activity of an expression product of the polypeptide or of the polypeptide, as between the test group and the control group.

The invention teaches a method for identifying a compound which inhibits or promotes the activity of a polypeptide, selected from the group consisting of SCD1, SCD2, SCD, G6PD, GPAT, FAS, COMT, ApoA-1, INSIG1, GLOL, A1BG, Spot-14 and delta-3, delta-2-enoyl-CoA isomerase, comprising the steps of: (a) selecting a test group having a host cell comprising the polypeptide wherein such host cell is heterologous to the polypeptide, a part thereof or an isolated polypeptide thereof and a control group; (b) treating the test group using a compound; and (c) determining the relative quantity or relative activity of a product of the polypeptide or of the polypeptide, as between the test group and the control group.

The invention teaches a method for identifying a compound which inhibits or promotes the activity of a human polynucleotide, wherein the human polynucleotide is a control region of a gene selected from the group consisting of G6PD, FAS, COMT, ApoA-1, INSIG1, Spot-14 and delta-3, delta-2-enoyl-CoA isomerase, comprising the steps of: (a) selecting a control animal having the human polynucleotide and a test animal having the human polynucleotide; (b) treating the test animal using a compound; and, (c) determining the relative quantity of an expression product of an operably linked polynucleotide to the human polynucleotide, as between the control animal and the test animal.

The invention teaches a method for identifying a compound which inhibits or promotes the activity of a human polynucleotide, wherein the human polynucleotide is a control region of a gene selected from the group consisting of G6PD, FAS, COMT, ApoA-1, INSIG1, Spot-14 and delta-3, delta-2-enoyl-CoA isomerase, comprising the steps of: (a) selecting a host cell comprising the human polynucleotide wherein such host cell is heterologous to the polynucleotide; (b) cloning the host cell and separating the clones into a test group and a control group; (c) treating the test group using a compound; and (d) determining the relative quantity of an expression product of an operably linked polynucleotide to the human polynucleotide, as between the test group and the control group.

The invention teaches a method for identifying a compound which inhibits or promotes the activity of a human polynucleotide, wherein the human polynucleotide is a control region of a gene selected from the group consisting of G6PD, FAS, COMT, ApoA-1, INSIG 1, Spot-14 and delta-3, delta-2-enoyl-CoA isomerase, comprising the steps of: (a) selecting a test group having a host cell comprising the human polynucleotide wherein such host cell is heterologous to the polynucleotide, a part thereof or an isolated polynucleotide thereof and a control group; (b) treating the test group using a compound; and (c) determining the relative quantity of an expression product of an operably linked polynucleotide to the human polynucleotide, as between the test group and the control group.

The invention teaches a composition for treating a disorder involving fatty acid regulated genes, the composition comprising a compound which modulates a polynucleotide from the coding sequence selected from the group consisting of SCD1, SCD2, SCD, G6PD, GPAT, FAS, COMT, ApoA-1, INSIG1, GLOL, Spot-14 and delta-3, delta-2-enoyl-CoA isomerase and a pharmaceutically acceptable carrier.

The invention teaches a composition for treating a fatty acid disorder comprising a compound which modulates a polypeptide selected from the group consisting of SCD1, SCD2, SCD, G6PD, GPAT, FAS, COMT, ApoA-1, INSIG1, GLOL, A1BG, Spot-14 and delta-3, delta-2-enoyl-CoA isomerase and a pharmaceutically acceptable carrier.

The invention teaches compositions for treating a fatty acid disorder comprising a compound which modulates a control region selected from the group consisting of G6PD, FAS, COMT, ApoA-1, INSIG1, Spot-14 and delta-3, delta-2-enoyl-CoA isomerase and a pharmaceutically acceptable carrier.

In another embodiment, the invention teaches a method for diagnosing the presence of or a predisposition for a disorder involving fatty acid regulated genes in a subject, the method comprising detecting a germline alteration in a polynucleotide representing the coding sequence selected from the group consisting of SCD1, SCD2, SCD, G6PD, GPAT, FAS, COMT, ApoA-1, INSIG1, GLOL, Spot-14 and delta-3, delta-2-enoyl-CoA isomerase in the subject, comprising comparing the germline sequence of the polynucleotide from a tissue sample from the subject with the germline sequence of a wild-type of the polynucleotide, wherein an alteration in the germline sequence of the subject indicates the presence of or a predisposition to the disorder.

The invention also teaches a method for diagnosing the presence of or a predisposition for a disorder involving fatty acid regulated genes in a subject, the method comprising detecting a germline alteration in a human polynucleotide representing the control region selected from the group consisting of G6PD, FAS, COMT, ApoA-1, INSIG1, Spot-14 and delta-3, delta-2-enoyl-CoA isomerase in the subject, comprising comparing the germline sequence of the human polynucleotide from a tissue sample from the subject with the germline sequence of a wild-type of the human polynucleotide, wherein an alteration in the germline sequence of the subject indicates the presence of or a predisposition to the disorder.

The invention also teaches a method for diagnosing the presence of or a predisposition for a disorder involving fatty acid regulated genes in a subject, the method comprising comparing the sequence of a polypeptide selected from the group consisting of SCD1, SCD2, SCD, G6PD, GPAT, FAS, COMT, ApoA-1, INSIG1, GLOL, A1BG, Spot-14 and delta-3, delta-2-enoyl-CoA isomerase from a tissue sample from the subject with the sequence of a wild-type of the polypeptide, wherein an alteration in the sequence of the subject as compared to the wild-type indicates the presence of or a predisposition to the disorder involving genes altered by fatty acids.

The invention further teaches a method for identifying a compound which inhibits or promotes a disorder involving fatty acid regulated genes, the method comprising the steps of: (a) selecting a control animal and a test animal both having a gene selected from the group consisting of SCD1, SCD2, SCD, G6PD, GPAT, FAS, COMT, ApoA-1, INSIG1, FTF1, GLOL, A1GB, Spot-14, delta-3, delta-2-enoyl-CoA isomerase, and METP or a control region sequence thereof; (b) treating the test animal using a compound; and, (c) determining the relative quantity of an expression product of the gene, as between the control animal and the test animal.

The invention also teaches a method for identifying a compound which inhibits or promotes a disorder involving fatty acid regulated genes, the method comprising the steps of: (a) selecting a host cell containing a gene selected from the group consisting of SCD1, SCD2, SCD, G6PD, GPAT, FAS, COMT, ApoA-1, INSIG1, FTF1, GLOL, A1GB, Spot-14, delta-3, delta-2-enoyl-CoA isomerase, and METP or a control region sequence thereof; (b) cloning the host cell and separating the clones into a test group and a control group; (c) treating the test group using a compound; and, (d) determining the relative quantity of an expression product of the gene, as between the test group and the control group.

The invention teaches a method for detecting the presence of or the predisposition for a disorder involving fatty acid regulated genes, the method comprising determining the level of expression of an expression product of a gene selected from a polynucleotide segment of the invention in a subject relative to a predetermined control level of expression, wherein a modified expression of the expression product as compared to the control is indicative of the presence of or the predisposition for a disorder involving genes altered by fatty acids.

The invention teaches a method for detecting the presence of or the predisposition for a disorder involving genes altered by fatty acids, the method comprising determining the level of expression of an expression product of a gene selected from the group consisting of SCD1, SCD2, SCD, G6PD, GPAT, FAS, COMT, ApoA-1, INSIG1, Spot-14, delta-3, delta-2-enoyl-CoA isomerase, FTF1, GLOL, A1BG and METP, in a subject relative to a predetermined control level of expression, wherein a modified expression of the expression product as compared to the control is indicative of the presence of or the predisposition for a disorder involving genes altered by fatty acids.

In another embodiment, the invention teaches an antibody immunoreactive with a polypeptide of the invention or an immunogenic portion thereof. In embodiments, the antibody is immunoreactive with a polypeptide selected from the group consisting of FTF1, GLOL and METP or an immunogenic portion thereof.

The invention teaches a method for screening a medium for a polypeptide of the invention or selected from the group consisting of FTF1, GLOL and METP, comprising: (a) labelling an antibody of the invention with a marker molecule to form a conjugate; (b) exposing the conjugate to the medium; and (c) determining whether there is binding between the conjugate and a biomolecule in the medium, wherein the binding indicates the presence of the polypeptide.

The invention teaches a method for screening a medium for a polypeptide of the invention or selected from the group consisting of FTF1, GLOL and METP, comprising: (a) exposing an antibody of the invention to the medium; (b) exposing the antibody to a marker molecule; and (c) determing whether there is binding between the marker molecule and a biomolecule in the medium, wherein the binding indicates the presence of the polypeptide.

The invention teaches a method for identifying genes or proteins regulated by fat, comprising: (a) selecting a species of animals and separating them into a test group and a control group; (b) feeding the test group and the control group a fat free diet for a period of time; (c) subsequently providing the test group enterally or parenterally with highly purified polyunsaturated fatty acids for a second period of time; (d) subsequently removing tissues from the control group and the test group; (e) comparing RNA from the tissues of the control group with tissues from the test group and selecting RNA which is expressed at a different level as between the test group and the control group; and (f) determining the genes or proteins associated with the selected RNA. The period of time may be at least 2 weeks. The second period of time is at least 2 weeks. The comparing may be by differential display and Northern blotting.

The invention teaches a method for identifying a compound which inhibits or promotes the activity of two or more human polynucleotides, wherein the human polynucleotides are control regions of genes selected from the group consisting of METP, GLOL, FTF1, A1BG, SCD, GPAT, G6PD, FAS, COMT, ApoA-1, INSIG1, Spot-14 and delta-3, delta-2-enoyl-CoA isomerase, the method comprising the steps of: (a) selecting two or more host cells comprising the human polynucleotides wherein such host cells are heterologous to the polynucleotides; (b) cloning the host cells and separating the clones into a test group and a control group; (c) treating the test group using a compound; and (d) determining the relative quantities of expression products of operably linked polynucleotides to the human polynucleotides, as between the test group and the control group.

The invention teaches a method for identifying a compound which inhibits or promotes the activity of two or more human polynucleotides, wherein the human polynucleotides are control regions of genes selected from the group consisting of METP, GLOL, FTF1, A1BG, SCD, GPAT, G6PD, FAS, COMT, ApoA-1, INSIG1, Spot-14 and delta-3, delta-2-enoyl-CoA isomerase, comprising the steps of: (a) selecting a test group having two or more host cells containing the human polynucleotides wherein such host cells are heterologous to the polynucleotides, parts thereof or isolated polynucleotides thereof and a control group; (b) treating the test group using a compound; and (c) determining the relative quantities of expression products of operably linked polynucleotides to the human polynucleotides, as between the test group and the control group.

The invention teaches a composition for treating a disorder involving fatty acid regulated genes comprising a compound which modulates two or more human polynucleotide control regions of genes selected from the group consisting of of METP, GLOL, FTF1, A1BG, SCD, GPAT, G6PD, FAS, COMT, ApoA-1, INSIG1, Spot-14 and delta-3, delta-2-enoyl-CoA isomerase and a pharmaceutically acceptable carrier.

The invention teaches a method for detecting the presence of or the predisposition for a disorder involving genes altered by fatty acids, the method comprising determining the level of expression of two or more expression products of genes selected from the group consisting of human SCD, G6PD, GPAT, FAS, COMT, ApoA-1, INSIG1, Spot-14, delta-3, delta-2-enoyl-CoA isomerase, FTF1, GLOL, A1BG and METP, in a subject relative to a predetermined control level of expression, wherein any modified expression of the expression products as compared to the control indicates the presence of or the predisposition for a disorder involving genes altered by fatty acids.

The comparing or determining of the invention may be performed by a method selected from the group consisting of immunoblotting, immunocytochemistry, enzyme-linked immunosorbent assay, DNA fingerprinting, in situ hybridization, polymerase chain reaction, reverse transcription polymerase chain reaction, radioimmunoassay, immunoradiometric assay and immunoenzymatic assay.

The compositions of the invention maybe selected from the group consisting of small organic molecules, peptides, polypeptides, antisense molecules, oligonucleotides, polynucleotides, fatty acids, antibodies of the invention, and functional and chemical derivatives thereof.

The invention teaches the use of a composition of the invention for treating a fatty acid disorder.

The disorder may be a PUFA disorder. The disorder may be selected from the group consisting of eczema, cardiovascular disorders (including but not limited to hypertriglyceridemia, dyslipidemia, atherosclerosis, coronary artery disease, cerebrovascular disease and peripheral vascular disease), inflammation (including but not limted to sinusitis, asthma, pancreatitis, osteoarthritis, rheumatoid arthritis and acne), body weight disorders (including but not limted to obesity, cachexia and anorexia), psychiatric disorders, cancer, cystic fibrosis, pre-menstrual syndrome, diabetes and diabetic complications.

### BRIEF DESCRIPTION OF THE DRAWINGS

The invention will now be described with reference to the drawings, wherein:

FIG. 1. FIG. 1 shows the nucleotide sequence and the amino acid sequence of the rat mitochondrial energy transfer protein, METP. These sequences correspond to SEQ. ID. Nos. 1 and 2.

FIG. 2. FIG. 2 shows the nucleotide sequence and the amino acid sequence of the human mitochondrial energy transfer protein, METP. These sequences correspond to SEQ. ID. Nos. 3 and 4.

FIG. 3. FIG. 3 shows a pairwise alignment between the rat (rMETP) and human (hMETP) mitochondrial energy transfer proteins. These sequences correspond to SEQ. ID. Nos. 2 and 4.

FIG. 4. FIG. 4 shows a multiple alignment among four members of the human mitochondrial energy transfer protein gene family, METP (SEQ. ID. No. 4), MCAT (mitochondrial carnitine/acylcarnitine translocase), TXTP (tricarboxylate transport protein) and UCP2 (mitochondrial uncoupling protein 2), highlighting in the box the PROSITE signature motif, PS00215, for this protein family.

FIG. 5. FIG. 5 shows a graph illustrating the Dense Alignment Surface (DAS) method prediction for transmembrane regions for the human METP.

FIG. 6. FIG. 6 shows the nucleotide sequence of the control region of the human METP gene, position −1 being the nucleotide immediately upstream of the ATG. This sequence corresponds to SEQ. ID. NO. 5.

FIG. 7. FIG. 7 shows the nucleotide sequence and the amino acid sequence of the rat glyoxalase II-like protein, GLOL. These sequences correspond to SEQ. ID. Nos. 6 and 7.

FIG. 8. FIG. 8 shows the nucleotide sequence and the amino acid sequence of the human glyoxalase II-like protein, GLOL. These sequences correspond to SEQ. ID. Nos. 8 and 9.

FIG. 9. FIG. 9 shows a pairwise alignment between the rat (rGLOL) and human (hGLOL) glyoxalase II-like proteins. These sequences correspond to SEQ. ID. Nos. 7 and 9.

FIG. 10. FIG. 10 shows a pairwise alignment between the human glyoxalase II-like protein (GLOL; SEQ. ID. NO. 9) and human glyoxalase II (GLO), indicating the high degree of similarity between these two proteins in domain one (underlined) and the invariant residues involved in metal ion coordination (marked by asterisks).

FIG. 11. FIG. 11 shows a three dimensional ribbon representation of the human glyoxalase II enzyme with the two zinc ions indicated as filled circles and domain one, the template upon which GLOL was modeled, surrounded by an oval.

FIG. 12. FIG. 12 shows a three dimensional ribbon representation of the modeled human GLOL.

FIG. 13. FIG. 13 shows the nucleotide sequence of the control region of the human GLOL gene, position −1 being the nucleotide immediately upstream of the ATG. This sequence corresponds to SEQ. ID. NO. 10.

FIG. 14. FIG. 14 shows the nucleotide sequence and the amino acid sequence of the rat fat responsive transcription factor, FTF1. These sequences correspond to SEQ. ID. Nos. 11 and 12.

FIG. 15. FIG. 15 shows the nucleotide sequence and the amino acid sequence of the human fat responsive transcription factor, FTF1. These sequences correspond to SEQ. ID. Nos. 13 and 14.

FIG. 16. FIG. 16 shows a multiple alignment among the mouse OASIS gene (MUS) and the rat (RAT) and human (HUM) FTF1 proteins. These sequences correspond to SEQ. ID. Nos. 12 and 14.

FIG. 17. FIG. 17 shows a three dimensional ribbon representation of c-fos and c-jun binding to a DNA template.

FIG. 18. FIG. 18 shows a three dimensional ribbon representation of c-fos and the modeled FTF1 binding to a DNA template.

FIG. 19. FIG. 19 shows the nucleotide sequence of the control region of the human FTF1 gene, position −1 being the nucleotide immediately upstream of the ATG. This sequence corresponds to SEQ. ID. Nos. 15.

FIG. 20. FIG. 20 shows the nucleotide sequence and the amino acid sequence of the rat alpha-1B-glycoprotein, A1BG. These sequences correspond to SEQ. ID. Nos. 16 and 17.

FIG. 21. FIG. 21 shows the nucleotide sequence and the amino acid sequence of the human alpha-1B-glycoprotein, A1BG. These sequences correspond to SEQ. ID. Nos. 18 and 19.

FIG. 22. FIG. 22 shows a pairwise alignment between the rat (rA1BG) and human (hA1BG) alpha-1B-glycoproteins. These sequences correspond to SEQ. ID. Nos. 17 and 19.

FIG. 23. FIG. 23 shows a three dimensional ribbon representation of the extracellular ligand-binding portion of the human killer cell inhibitory receptor, KIR2DL2.

FIG. 24. FIG. 24 shows a three dimensional ribbon representation of the modeled fourth and fifth domains of the human A1BG.

FIG. 25. FIG. 25 shows the nucleotide sequence of the control region of the human A1BG gene, position −1 being the nucleotide immediately upstream of the ATG. This sequence corresponds to SEQ. ID. NO. 20.

FIG. 26. FIG. 26 shows the nucleotide sequence of the control region of the human SCD gene, position −1 being the nucleotide immediately upstream of the ATG. This sequence corresponds to SEQ. ID. NO. 21.

FIG. 27. FIG. 27 shows a comparison of the polyunsaturated fatty acids response region (PUFA-RR) among the promoters for the mouse SCD1 (mSCD1), the mouse SCD2 (mSCD2) and the human SCD (hSCD; SEQ. ID. Nos. 22 and 23), position −1 being the nucleotide immediately upstream of the ATG.

FIG. 28. FIG. 28 shows the nucleotide sequence and the amino acid sequence of the human glycerol-3-phosphate acyltransferase, hGPAT. These sequences correspond to SEQ. ID. Nos. 24 and 25.

FIG. 29. FIG. 29 shows a multiple alignment among the human (hGPAT), the rat (rGPAT) and mouse (mGPAT) glycerol-3-phosphate acyltransferase proteins. This sequence corresponds to SEQ. ID. NO. 25.

FIG. 30. FIG. 30 shows the nucleotide sequence of the control region of the human GPAT gene, position −1 being the nucleotide immediately upstream of the ATG. This sequence corresponds to SEQ. ID. NO. 26.

### DETAILED DESCRIPTION OF EMBODIMENTS

It is well established that fatty acids are important in maintaining cellular membranes and processes. They are also seen as having important, yet poorly understood roles in human disease. Since the clinical effects of fatty acids are so variable, the present inventors postulated that fatty acids could be regulating specific genes in certain disease conditions. To date, little is known about how fatty acids regulate genes or gene expression.

The present inventors used a fat free dietary model supplemented with various PUFAs, to elicit tissue fatty acid changes. By using this dietary model and differential display techniques, the present inventors identified genes which are regulated by fatty acids. Some of these identified genes are novel while some are well known. The inventors' discovery of fat regulated genes leads to a better understanding of why certain diseases can be treated more effectively with fatty acids. The present invention thus teaches the development of compounds for the treatment of human or mammalian fatty acid metabolism disorders.

Drugs that modify fatty acid metabolism should produce more dramatic changes in fatty acid levels than would be possible through dietary intervention. This enables the development of effective therapeutics which would not be possible using fatty acids as drugs.

Definitions

The following illustrative explanations are provided to facilitate understanding of certain terms used frequently herein. The explanations are provided as a convenience and are not limitative of the invention.

Agonist refers to any molecule or pharmaceutical agent, such as a drug or hormone, which enhances the activity of another molecule.

Antagonist refers to any molecule or pharmaceutical agent, such as a drug or hormone, which inhibits or extinguishes the activity of another molecule.

Chemical Derivative. As used herein, a molecule is said to be a “chemical derivative” of another molecule when it contains additional chemical moieties not normally a part of the molecule. Such moieties can improve the molecule's solubility, absorption, biological half life, and the like. The moieties can alternatively decrease the toxicity of the molecule, eliminate or attenuate any undesirable side effect of the molecule, and the like. Moieties capable of mediating such effects are disclosed in Mack E. W., 1990, Remington's Pharmaceutical Sciences, Mack Publishing Company, Easton, Pa., 13^{th} edition

Procedures for coupling such moieties to a molecule are well known in the art.

Compositions include genes, proteins, polynucleotides, peptides, compounds, drugs, and pharmacological agents.

Control region refers to a nucleic acid sequence capable of, or required for, assisting or impeding initiation, termination, or otherwise regulating the transcription of a gene. The control region may include a promoter, enhancer, silencer and/or any other regulatory element. A control region also includes a nucleic acid sequence that may or may not be independently or exclusively sufficient to initiate, terminate, or otherwise regulate transcription, however, is capable of effecting such regulation in association with other nucleic acid sequences.

Desaturase refers to a fatty acid desaturase, which is an enzyme capable of generating a double bond in the hydrocarbon region of a fatty acid molecule.

Disorder as used herein refers to derangement or abnormality of structure or function. Disorder includes disease.

Drug. Drugs include, but are not limited to proteins, peptides, degenerate peptides, agents purified from conditioned cell medium, organic molecules, inorganic molecules, antibodies or oligonucleotides. The drug can be naturally occurring or synthetically or recombinantly produced.

Enhancer is a nucleic acid sequence comprising a DNA regulatory element that enhances or increases transcription when bound by a specific transcription factor or factors. Moreover, an enhancer may function in either orientation and in any location (upstream or downstream relative to the promoter) to effect and generate increased levels of gene expression when bound by specific factors. In addition, according to the present invention, an enhancer also refers to a compound (i.e. test compound) that increases or promotes the enzymatic activity of the fatty acid regulated gene, and/or increases or promotes the transcription of the gene.

Fatty Acids are a class of compounds comprising a long saturated or mono or polyunsaturated hydrocarbon chain and a terminal carboxyl group.

Functional Derivative. A “functional derivative” of a sequence, either protein or nucleic acid, is a molecule that possesses a biological activity (either functional or structural) that is substantially similar to a biological activity of the protein or nucleic acid sequence. A functional derivative of a protein can contain post-translational modifications such as covalently linked carbohydrate, depending on the necessity of such modifications for the performance of a specific function. The term “functional derivative” is intended to include the “fragments,” “segments,” “variants,” “analogs,”or “chemical derivatives” of a molecule.

Gene refers to a nucleic acid molecule or a portion thereof, the sequence of which includes information required for the production of a particular protein or polypeptide chain. The polypeptide can be encoded by a full-length sequence or any portion of the coding sequence, so long as the functional activity of the protein is retained. A gene may comprise regions preceding and following the coding region as well as intervening sequences (introns) between individual coding segments (exons). A “heterologous” region of a nucleic acid construct (i.e. a heterologous gene) is an identifiable segment of DNA within a larger nucleic acid construct that is not found in association with the other genetic components of the construct in nature. Thus, when the heterologous gene encodes a mammalian fatty acid regulated gene, the gene will usually be flanked by a promoter that does not flank the structural genomic DNA in the genome of the source organism.

Host system may comprise a cell, tissue, organ, organism or any part thereof, which provides an environment or conditions that allow for, or enable, transcription and/or transcription.

Identity, similarity, homology or homologous, refer to relationships between two or more polynucleotide sequences, as determined by comparing the sequences. In the art, identity also means the degree of sequence relatedness between polynucleotide sequences, as the case may be, as determined by the match between strings of such sequences. Both identity and similarity can be readily calculated (Lesk A. M., ed., 1988, Computational Molecular Biology, Oxford University Press, NY; Smith D. W., ed., 1993, Biocomputing: Informatics and Genome Project, Academic Press, NY; Griffin A. M. and Griffin H. G., eds., 1994, Computer Analysis of Sequence Data, Part 1, Humana Press, NJ; von Heijne G., 1987, Sequence Analysis in Molecular Biology, Academic Press, NY and Gribskov M. and Devereux J., eds., 1991, Sequence Analysis Primer, M Stockton Press, NY). While there exist a number of methods to measure identity and similarity between two polynucleotide sequences, both terms are well known to skilled artisans (von Heijne G., 1987, Sequence Analysis in Molecular Biology, Academic Press, NY; Gribskov M. and Devereux J., eds., 1991, Sequence Analysis Primer, M Stockton Press, NY; Carillo H. and Lipman D., 1988, SIAM J. Applied Math., 48: 1073). Methods commonly employed to determine identity or similarity between sequences include, but are not limited to those disclosed in Carillo H. and Lipman D., 1988, SIAM J. Applied Math., 48: 1073. Methods to determine identity and similarity are codified in computer programs. Computer program methods to determine identity and similarity between two sequences include, but are not limited to, GCG program package (Devereux et al., 1984, Nucl. Acid Res., 12: 387-395), BLASTP, BLASTN and FASTA (Altschul et al., 1990, J. Molec. Biol., 215: 403-410).

Isolated means altered “by the hand of man” from its natural state; i.e., that, if it occurs in nature, it has been changed or removed from its original environment, or both. For example, a naturally occurring polynucleotide naturally present in a living organism in its natural state is not “isolated,” but the same polynucleotide separated from coexisting materials of its natural state is “isolated”, as the term is employed herein. As part of or following isolation, such polynucleotides can be joined to other polynucleotides, such as DNA, for mutagenesis, to form fusion proteins, and for propagation or expression in a host, for instance. The isolated polynucleotides, alone or joined to other polynucleotides such as vectors, can be introduced into host cells, in culture or in whole organisms. Introduced into host cells in culture or in whole organisms, such DNA still would be isolated, as the term is used herein, because they would not be in their naturally occurring form or environment. Similarly, the polynucleotides may occur in a composition, such as a media formulations, solutions for introduction of polynucleotides, for example, into cells, compositions or solutions for chemical or enzymatic reactions, for instance, which are not naturally occurring compositions, and, therein remain isolated polynucleotides within the meaning of that term as it is employed herein.

Mutation. A “mutation” is any detectable change in the genetic material. A mutation can be any (or a combination of) detectable, unnatural change affecting the chemical or physical constitution, mutability, replication, phenotypic function, or recombination of one or more deoxyribonucleotides; nucleotides can be added, deleted, substituted for, inverted, or transposed to new positions with and without inversion. Mutations can occur spontaneously and can be induced experimentally by application of mutagens or by site-directed mutagenesis. A mutant polypeptide can result from a mutant nucleic acid molecule.

Nucleic acid construct refers to any genetic element, including, but not limited to, plasmids and vectors, that incorporate polynucleotide sequences. For example, a nucleic acid construct may be a vector comprising a promoter or control region that is operably linked to a heterologous gene.

Operably linked as used herein indicates the association of a promoter or control region of a nucleic acid construct with a heterologous gene such that the presence or modulation of the promoter or control region influences the transcription of the heterologous gene, including genes for reporter sequences. Operably linked sequences may also include two segments that are transcribed onto the same RNA transcript. Thus, two sequences, such as a promoter and a “reporter sequence” are operably linked if transcription commencing in the promoter produces an RNA transcript of the reporter sequence.

Plasmids. Starting plasmids disclosed herein are either commercially available, publicly available, or can be constructed from available plasmids by routine application of well known, published procedures. Many plasmids and other cloning and expression vectors that can be used in accordance with the present invention are well known and readily available to those of skill in the art. Moreover, those of skill readily may construct any number of other plasmids suitable for use in the invention.

Polynucleotides(s) of the present invention may be in the form of RNA, such as mRNA, or in the form of DNA, including, for instance, cDNA and genomic DNA obtained by cloning or produced by chemical synthetic techniques or by a combination thereof. The DNA may be double-stranded or single-stranded. Single-stranded polynucleotides may be the coding strand, also known as the sense strand, or it may be the non-coding strand, also referred to as the anti-sense strand. Polynucleotides generally refers to any polyribonucleotide or polydeoxribonucleotide, which may be unmodified RNA or DNA or modified RNA or DNA. Thus, for instance, polynucleotides as used herein refers to, among others, single-and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded, or a mixture of single- and double-stranded regions. In addition, polynucleotide as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term polynucleotide also includes DNA or DNA that contain one or more modified bases. Thus, DNA or DNA with backbones modified for stability or for other reasons are “polynucleotides” as that term is intended herein. Moreover, DNA or DNA comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including simple and complex cells, inter alia. Polynucleotides embraces short polynucleotides often referred to as oligonucleotide(s). It will also be appreciated that RNA made by transcription of this doubled stranded nucleotide sequence, and an antisense strand of a nucleic acid molecule of the invention or an oligonucleotide fragment of the nucleic acid molecule, are contemplated within the scope of the invention. An antisense sequence is constructed by inverting the sequence of a nucleic acid molecule of the invention, relative to its normal presentation for transcription. Preferably, an antisense sequence is constructed by inverting a region preceding the initiation codon or an unconserved region. The antisense sequences may be constructed using chemical synthesis and enzymatic ligation reactions using procedures known in the art.

Promoter refers to a nucleic acid sequence comprising a DNA regulatory element capable of binding RNA polymerase directly or indirectly to initiate transcription of a downstream (3′ direction) gene. In accordance with the present invention, a promoter of a nucleic acid construct that includes a nucleotide sequence, wherein the nucleotide sequence may be linked to a heterologous gene such that the induction of the promoter influences the transcription of the heterologous gene.

Purified. A “purified” protein or nucleic acid is a protein or nucleic acid preparation that is generally free of contaminants, whether produced recombinantly, chemically synthesized or purified from a natural source.

Recombinant refers to recombined or new combinations of nucleic acid sequences, genes, or fragments thereof which are produced by recombinant DNA techniques and are distinct from a naturally occurring nucleic acid sequence

Regulatory element refers to a deoxyribonucleotide sequence comprising the whole, or a portion of, a nucleic acid sequence to which an activated transcriptional regulatory protein, or a complex comprising one or more activated transcriptional regulatory proteins, binds so as to transcriptionally modulate the expression of an associated gene or genes, including heterologous genes.

Reporter gene is a nucleic acid coding sequence whose product is a polypeptide or protein that, is not otherwise produced by the host cell or host system, or which is produced in minimal or negligible amounts in the host cell or host system, and which is detectable by various known methods such that the reporter gene product may be quantitatively assayed to analyse the level of transcriptional activity in a host cell or host system. Examples include genes for luciferase, chloramphenicol acetyl transferase (CAT), beta-galactosidase, secreted placental alkaline phosphatase and other secreted enzymes.

Silencer refers to a nucleic acid sequence or segment of a DNA control region such that the presence of the silencer sequence in the region of a target gene suppresses the transcription of the target gene at the promoter through its actions as a discrete DNA segment or through the actions of trans-acting factors that bind to these genetic elements and consequently effect a negative control on the expression of a target gene.

Stringent hybridization conditions are those which are stringent enough to provide specificity, reduce the number of mismatches and yet are sufficiently flexible to allow formation of stable hybrids at an acceptable rate. Such conditions are known to those skilled in the art and are described, for example, in Sambrook et al., 1989, Molecular Cloning, 2nd Edition, Cold Spring Harbor Laboratory Press, Cold Spring Harbour, N.Y. or Ausubel et al., 1994-, Current Protocols in Molecular Biology, John Wiley & Sons, NY. By way of example only, stringent hybridization with short nucleotides may be carried out at 5-10° C. below the T_{M} using high concentrations of probe such as 0.01-1.0 pmole/ml. Preferably, the term “stringent conditions” means hybridization will occur only if there is at least 95% and preferably at least 97% identity between the sequences.

Tag refers to a specific short amino acid sequence, or the oligonucleotide sequence that encodes it, wherein said amino acid or nucleic acid sequence may comprise or encode, for example, a c-myc epitope and/or a string of six histidine residues recognizable by commercially available antibodies. In practice, a tag facilitates the subsequent identification and purification of a tagged protein.

Tagged protein as used herein refers to a protein comprising a linked tag sequence. For example, a tagged protein includes a mammalian fatty acid regulated polypeptide linked to a c-myc epitope and six histidine residues at the carboxyl terminus of the amino acid sequence.

Test compounds as used herein encompass small molecules (e.g. small organic molecules), pharmacological compounds or agents, peptides, proteins, antibodies or antibody fragments, and nucleic acid sequences, including DNA and RNA sequences.

Transfection refers to a process whereby exogenous or heterologous DNA (i.e. a nucleic acid construct) is introduced into a recipient eukaryotic host cell. Therefore, in eukaryotic cells, the acquisition of exogenous DNA into a host cell is referred to as transfection. In prokaryotes and eukaryotes (for example, yeast and mammalian cells) introduced DNA may be maintained on an episomal element such as a plasmid or integrated into the host genome. With respect to eukaryotic cells, a stably transfected cell is one in which the introduced DNA has become integrated into a chromosome so that it is inherited by daughter cells through chromosome replication. This stability is demonstrated by the ability of the eukaryotic cell to establish cell lines or clones comprised of a population of daughter cells containing the introduced DNA.

Transformation refers to a process whereby exogenous or heterologous DNA (i.e. a nucleic acid construct) is introduced into a recipient prokaryotic host cell. Therefore, in prokaryotic cells, the acquisition of exogenous DNA into a host cell is referred to as transformation. Transformation in eukaryotes refers to the conversion or transformation of eukaryotic cells to a state of unrestrained growth in culture, resembling a tumorigenic condition. In prokaryotes and eukaryotes (for example, yeast and mammalian cells) introduced DNA may be maintained on an episomal element such as a plasmid or integrated into the host genome. With prokaryotic cells, a stably transformed bacterial cell is one in which the introduced DNA has become integrated into a chromosome so that it is inherited by daughter cells through chromosome replication. This stability is demonstrated by the ability of the prokaryotic cell to establish cell lines or clones comprised of a population of daughter cells containing the introduced DNA.

Transfection/transformation as used herein refers to a process whereby exogenous or heterologous DNA (e.g. a nucleic acid construct) has been introduced into a eukaryotic or prokaryotic host cell or into a host system.

Variant(s) of polynucleotides are polynucleotides that differ in nucleotide sequence from another, reference polynucleotide. A “variant” of a protein or nucleic acid is meant to refer to a molecule substantially similar in structure and biological activity to either the protein or nucleic acid. Thus, provided that two molecules possess a common activity and can substitute for each other, they are considered variants as that term is used herein even if the composition or secondary, tertiary, or quaternary structure of one of the molecules is not identical to that found in the other, or if the amino acid or nucleotide sequence is not identical. Generally, differences are limited so that the nucleotide sequences of the reference and the variant are closely similar overall and, in many regions, identical. Changes in the nucleotide sequence of the variant may be silent. That is, they may not alter the amino acids encoded by the polynucleotide. Where alterations are limited to silent changes of this type a variant will encode a polypeptide or polynucleotide with the same amino acid sequence as the reference. Changes in the nucleotide sequence of the variant may alter the amino acid sequence of a polypeptide encoded by the reference polynucleotide. Such nucleotide changes may result in amino acid substitutions, additions, deletions, fusions and truncations in the polypeptide or polynucleotide encoded by the reference sequence.

Vector. A plasmid or phage DNA or other DNA sequence into which DNA can be inserted to be cloned. The vector can replicate autonomously in a host cell, and can be further characterized by one or a small number of endonuclease recognition sites at which such DNA sequences can be cut in a determinable fashion and into which DNA can be inserted. The vector can further contain a marker suitable for use in the identification of cells transformed with the vector. Markers, for example, are tetracycline resistance or ampicillin resistance. The words “cloning vehicle” are sometimes used for “vector.”

As hereinbefore mentioned, the present inventors have identified and sequenced various DNA sequences encoding genes associated with fatty acid metabolism and their promoters, herein referred to as “subject polynucleotide(s)”. The proteins, peptides and polypeptides produced by or expressed from the subject polynucleotides are herein referred to as “subject polypeptides.”

It will be appreciated that the invention includes nucleotide or amino acid sequences which have substantial sequence homology with, respectively, the subject polynucleotides or polypeptides. The term “sequences having substantial sequence homology” means those nucleotide and amino acid sequences which have slight or inconsequential sequence variations from the subject polynucleotides; i.e. the homologous sequences function in substantially the same manner to produce substantially the same polypeptides as the actual sequences. The variations may be attributable to local mutations or structural modifications. It is expected that a sequence having 85-90% sequence homology with the DNA sequence of the invention will provide functional subject polypeptides which retain substantially the same biological function or activity as the polynucleotide encoded by the subject polynucleotides. Further embodiments of the invention are polynucleotides that are at least 70% identical over their entire length to a subject polynucleotide, and polynucleotides which are complementary to such polynucleotides. Other embodiments are polynucleotides that comprise a region that is at least 80% identical over their entire length to a subject polynucleotide and polynucleotides complementary thereto. This includes polynucleotides at least 90% identical over their entire length to the same, and among these embodiments are polynucleotides with at least 95% homology. Furthermore, those with at least 97% are highly preferred among those with at least 95%, and among these those with at least 98% and at least 99% are particularly highly preferred, with at least 99% being the more preferred.

Differential Gene Expressions in Liver of Rats Fed Diets Supplemnented with PUFAs

The present inventors have identified genes that were modulated or regulated by dietary fat using a differential display technique (Liang P. and Pardee A. B., 1992, Science, 257: 967-971). Groups of female Wistar rats were fed fat free diet for two weeks. After two weeks all groups but one (controls) were supplemented with highly purified PUFAs for an additional two weeks. The rats were sacrificed and tissues were removed to obtain RNA for differential display and Northern blotting. Differential display identified genes regulated by dietary fat by comparing mRNA populations from rat liver.

For the study, the rats were divided into six groups and fed fat free diet for 14 days. Each group was fed fat free diet for an additional two weeks with the following supplements: group 1—no supplement (fat free control); group 2—linoleic acid (LA, 18:2n-6); group 3—gamma-linolenic acid (GLA, 18:3n-6); group 4—alpha-linolenic acid (ALA, 18:3n-3); group 5—eicosapentaenoic acid (EPA, 20:5n-3) and group 6—docosahexaenoic acid (DHA, 22:6n-3). Fatty acids were administered in the diet at a fatty acid-specific concentration of 5% (w/w), as described in Example 1. The rats were fed ad libitum.

The protocol used in the present differential display experiment is based on, and closely followed, the Clontech Delta Differential Display Kit (Clontech Catalog No. K1810-1). Each primer combination was used with reverse-transcribed cDNA from three rats from each treatment group and all experiments were done independently in duplicate.

The inventors' preliminary differential display studies showed no differences between control and LA-treated groups. These previous studies along with other technical reasons led the present inventors to choose groups 1 (no supplement, fat free control), 3 (GLA) and 6 (DHA) for the complete differential display screen.

Rat liver cDNA was amplified using 90 combinations of 10 arbitrary P primers and 9 anchored T primers as well as 55 combinations of P primers only. Control, GLA and DHA samples were prepared in triplicate for a total of nine samples for each primer pair. Typically, reactions for five primer pairs were carried out at the same time. These samples were run together on a polyacrylamide gel. The experiment was repeated to verify the results. A total of 2610 individual reactions were performed and analyzed. Only bands which were differentially expressed in both experiments were further characterized.

Results

Inspection of the differential display gels revealed 144 bands that appeared to be differentially expressed in at least two independent experiments. Each experiment included three rats from each of the diet groups 1, 3 and 6. A total of 84 unique bands, being the PCR products of P and T primers, were identified as well as an additional 60 duplicate bands apparently resulting from an amplification reaction using only one of the two primers present in the reaction. One hundred of the 144 differentially displayed hits were cloned into pCRII (Invitrogen) using the TA-cloning technique, and used for Northern blot analyses. The 100 Northern blots resulted in the confirmation of 61 clones which were differentially expressed and 39 clones which were not. The Northern blot membranes contained rat liver RNA isolated from the control, LA-, GLA-, EPA- and DHA-treated animal groups.

Subsequent sequencing of the 61 positively-identified clones on Northern blots led to the identification of 28 unique DNA sequences belonging to 14 different genes. The mRNA levels of eleven of these genes are higher under fat free conditions and reduced when fed PUFAs while the mRNA level of three of these genes are lower under fat free conditions compared to PUFA supplementation. These 14 genes are described in Tables 1 and 2.

Genes Discovered

I. METP Gene aid Polypeptide

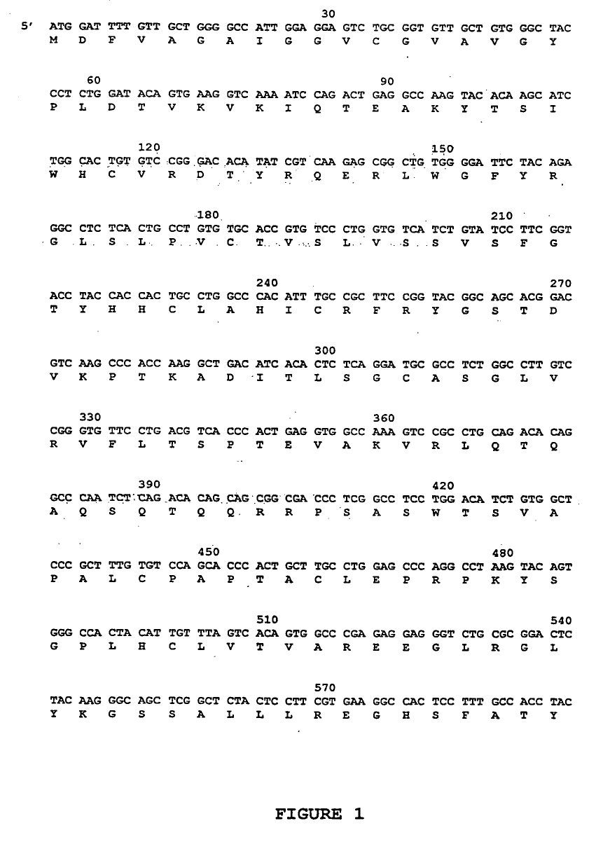

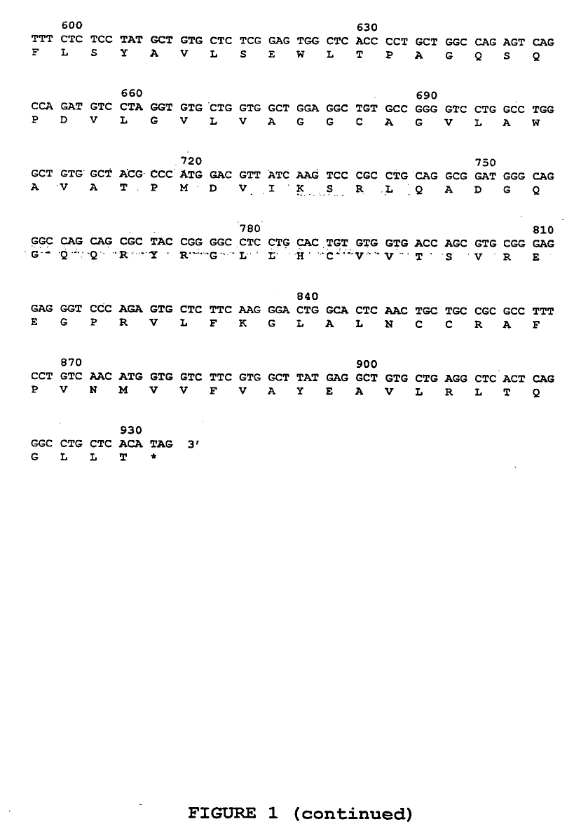

One of the differentially expressed bands identified during the study was a rat partial cDNA clone identified as belonging to an uncharacterized and unknown mitochondrial carrier protein gene, which was designated METP (mitochondrial energy transfer protein). Using GeneTrapper™ technology (Gibco BRL), the rat METP gene was cloned. The full length coding sequence (CDS) was determined by DNA sequencing (FIG. 1).

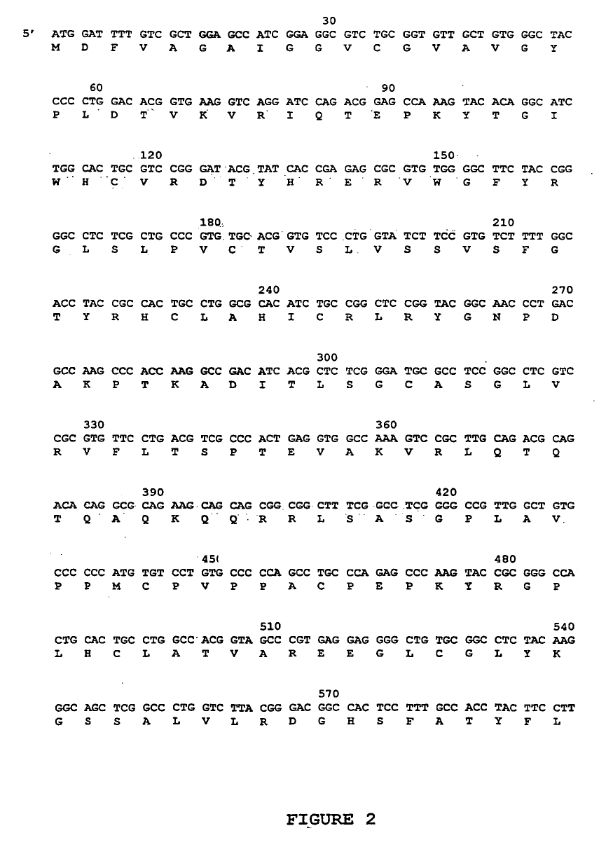

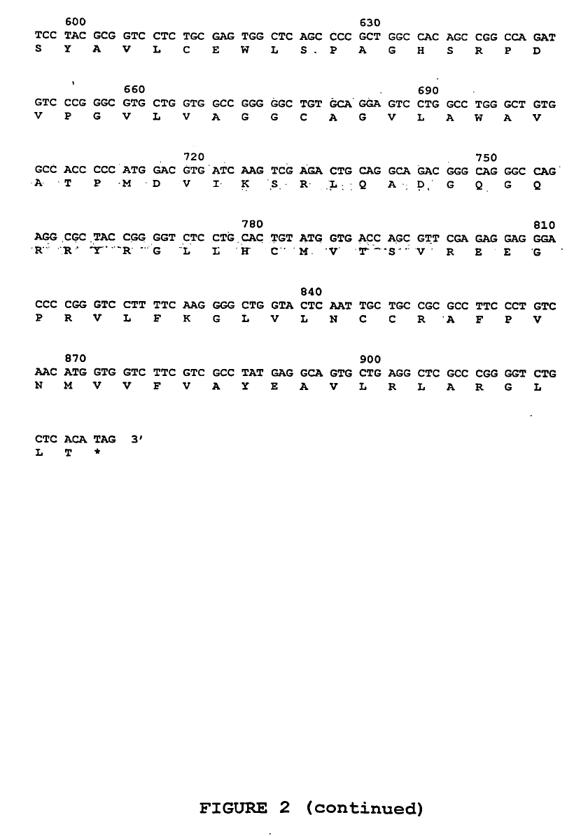

Using bioinformatic techniques, the human gene was located in GenBank's HTGS database on a fragment of genomic DNA from chromosome 14 (GenBank Accession No. AL135838). Oligonucleotide primers were synthesized and the proposed human gene sequence was verified by cloning and DNA sequencing (FIG. 2). Exons were mapped onto genomic DNA from AL135838 and the gene was found to comprise 6 coding exons.

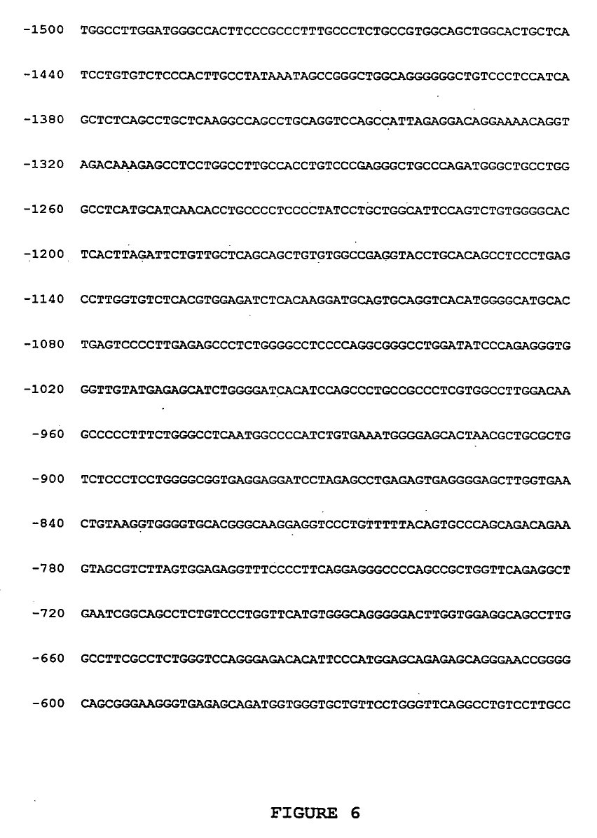

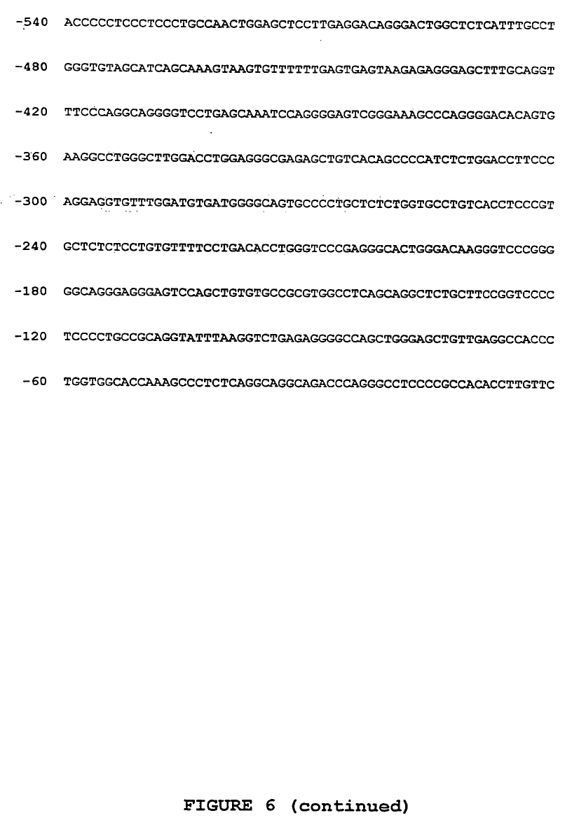

Using bioinformatic techniques, the control region of the human METP gene was identified and mapped out. The control region between positions-1 and -1500 from the ATG is shown in FIG. 6.

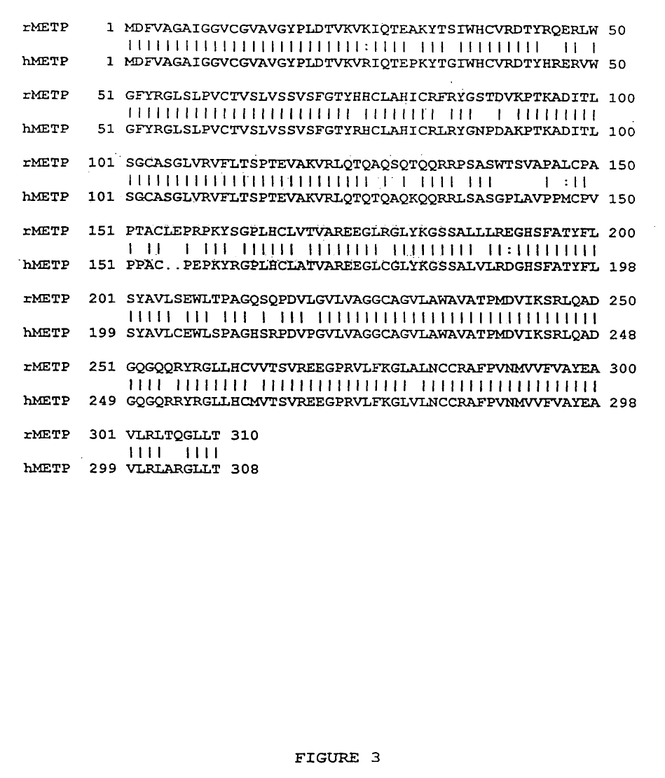

The human and rat gene were compared and found to be 87% identical (89% positive) at the amino acid sequence level (FIG. 3).

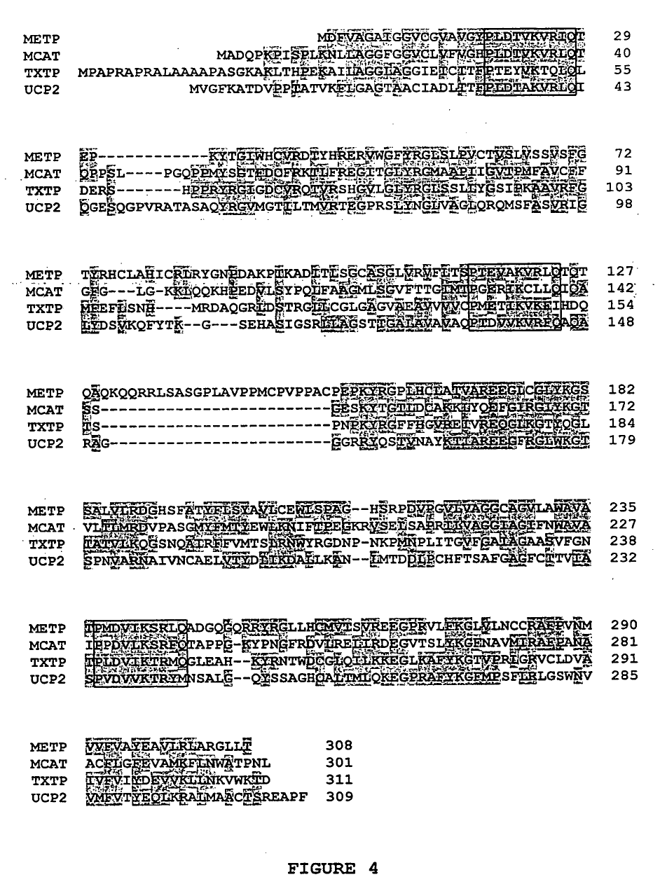

The human and rat proteins were found to belong to the mitochondrial energy transfer (carrier) protein family and contain 3 signature motifs which are characteristic of this protein family. FIG. 4 shows a multiple alignment among four members of the human mitochondrial energy transfer protein gene family, METP, MCAT (mitochondrial carnitine/acylcarnitine translocase; Genbank Accession Number NP_{—}000378), TXTP (tricarboxylate transport protein; Genbank Accession Number P53007) and UCP2 (mitochondrial uncoupling protein 2; Genbank Accession Number NP_{—}003346), highlighting in the box the PROSITE signature motif, PS00215, for this protein family. The motifs for the human protein are located at amino acid positions 19-28, 115-124 and 237-246 (boxed areas in FIG. 4). The consensus pattern for the motif is P-x-[DE]-x-[LIVAT]-[RK]-x-[LRH]-[LIVMFY]-[QGAIVM] (Prosite pattern PS00215).

A multiple sequence alignment showing the relationship among METP and closely related human proteins of the mitochondrial carrier protein family is shown in FIG. 4.

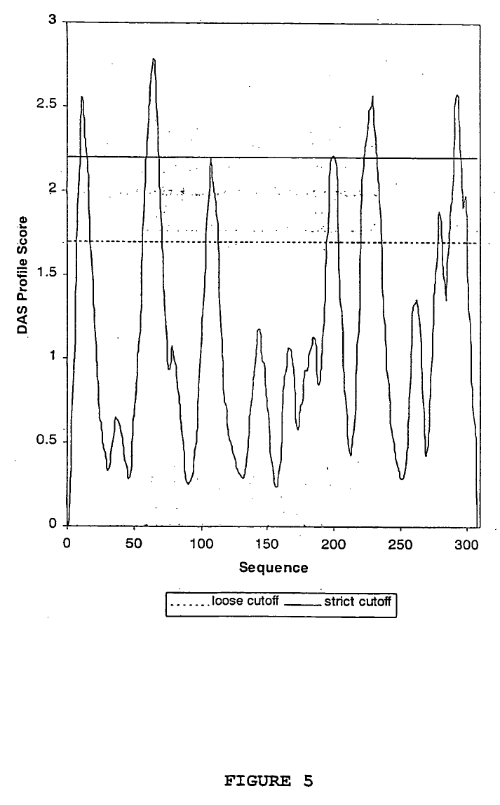

The rat and human METP protein has a tripartite domain structure, which is indicative of this family of proteins. A graphic of the dot matrix analysis of the protein sequence was generated (data not shown). Each of the internally similar domains contains 2 transmembrane regions and a short loop. This is evident based on the Dense Alignment Surface (DAS) prediction (Cserzo et al., 1997, Prot. Eng., 10: 673-676) result shown in FIG. 5 for the human gene where the six peaks reaching the strict cutoff limit are the predicted transmembrane regions.

Using the rat partial cDNA clone as the probe for Northern blotting, the relative abundance of mRNA for this gene in rat liver was shown to be dramatically increased after PUFA treatment (Tables 1 and 2).

Northern blot studies evaluating tissue distribution (see Example 37) in human and rat showed that METP transcript is only detected in liver.

These observations confirm that METP is a liver specific mitochondrial carrier protein regulated by polyunsaturated fatty acids. The present inventors' data suggest that mRNA abundance is increased in STZ-induced diabetic rats. Thus, the present invention describes a novel drug target, i.e. METP, which includes the use of the gene as well as the promoter to screen for novel drugs or chemical entities that modulate expression of the gene or activity of the gene product.

II. GLOL Gene and Polypeptide

Another of the differentially expressed bands identified during the study was a rat partial cDNA clone highly similar to a human gene of unknown function. This human gene is referred to as a “mRNA expressed in thyroid gland” in GenBank Accession Nos. D83198 and NM_{—}014297. Although the CDS for this human gene sequence has been submitted to GenBank there are no existing publications for it.

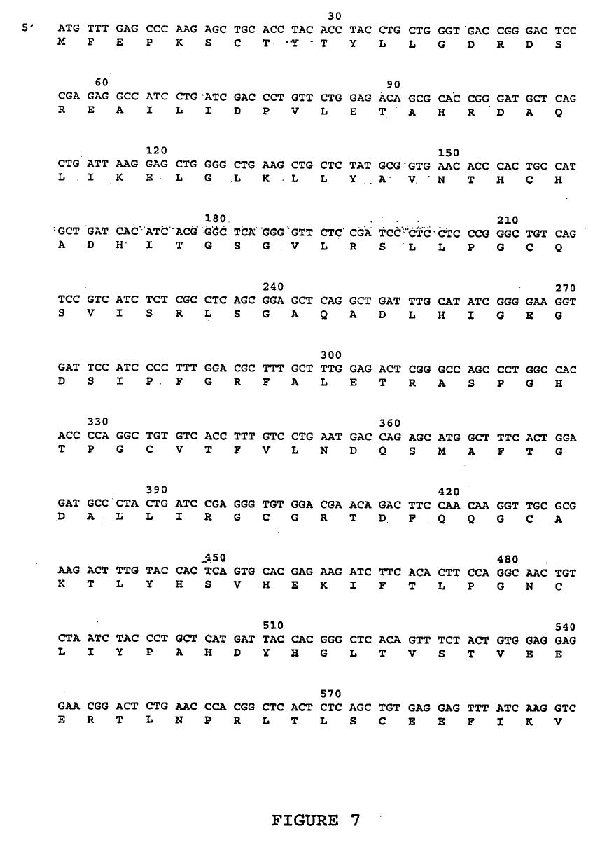

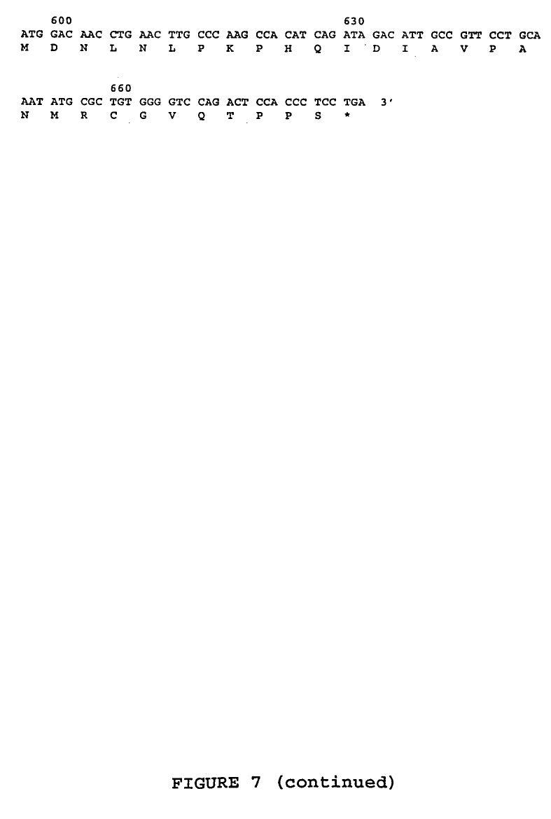

GenBank's EST division was searched using BLASTN to identify rodent sequences containing the 5′ end of the gene. Using a forward primer designed from one such identified mouse EST (GenBank Accession No. AW106717) and a reverse primer from the cloned rat partial cDNA, the fill length coding sequence (CDS) of the rat GLOL (glyoxalase II-like) gene was determined. The sequence is shown in FIG. 7.

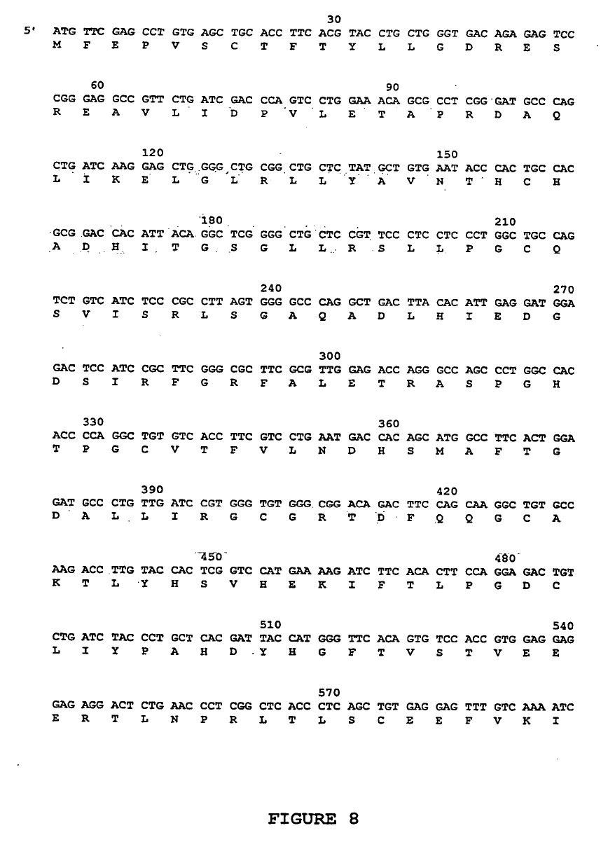

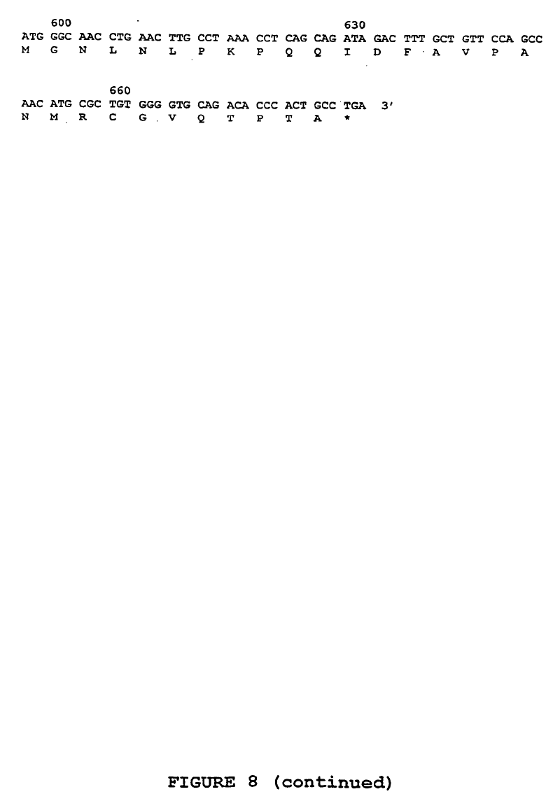

The human sequence in GenBank was verified by PCR cloning (using primers designed from D83198) and DNA sequencing (FIG. 8).

Using bioinformatic techniques, the human gene was located in GenBank's HTGS division on a sequence assigned to chromosome 19 (GenBank Accession No. AC068785). Exons were mapped onto this genomic DNA and the gene was found to contain 6 coding exons.

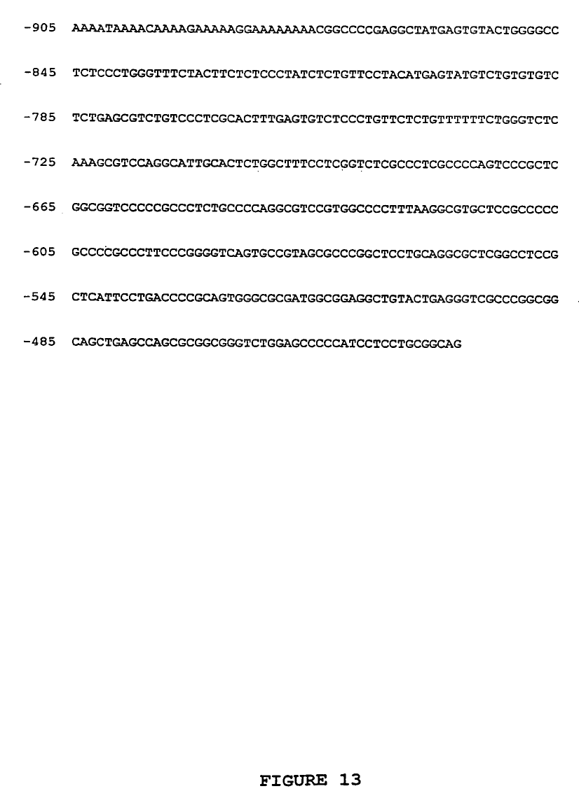

Using bioinformatic techniques, the control region of the human GLOL gene was identified and mapped out. By searching GenBank's EST division using BLASTN with the human GLOL CDS, a collection of human ESTs containing 5′ UTR for the gene was identified. This experiment revealed the presence of an upstream intron 437 bp long. The present inventors' results differed from that listed in GenBank Accession No. D83198 in which the end of this intron is incorrectly identified as 5′ UTR. The present inventors further identified an area containing highly repetitive elements upstream from the -905 position. The control region between positions-438 and -905 from the ATG was identified and is shown in FIG. 13.

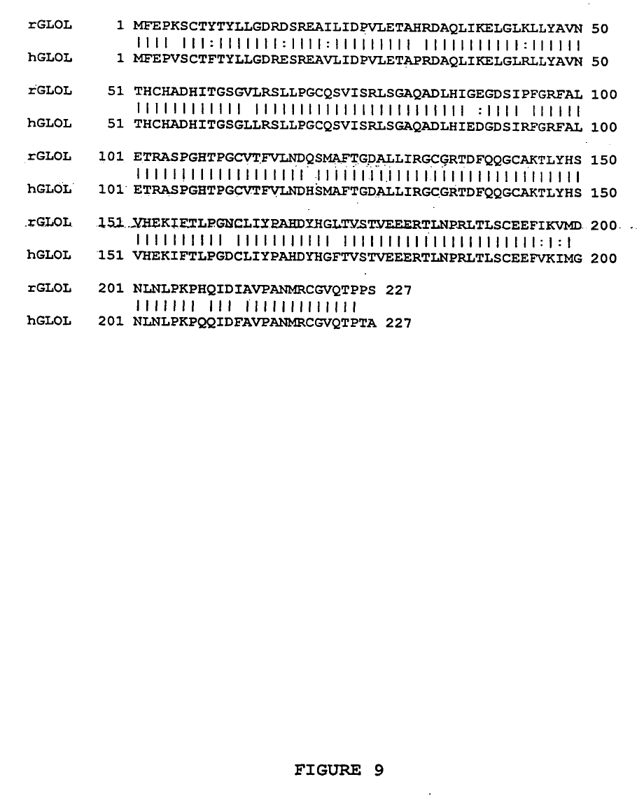

The amino acid sequences for the rat and the human genes were compared and found to be 92% identical or 95% similar (FIG. 9).

The present inventors determined that GLOL is a member of the metallo-beta-lactamase superfamily of proteins. The protein is composed of two domains. Domain 1 comprises residues 1-168 and has homology to other metallo-beta-lactamases, while domain 2 comprises residues 169-227. Apart from the beta-lactamases a number of other proteins belong to this family. These proteins include thiolesterases, members of the glyoxalase II family that catalyse the hydrolysis of S-D-lactoyl-glutathione to form glutathione and D-lactic acid and a competence protein that is essential for natural transformation in Neisseria gonorrhoeae and appears to be a transporter involved in DNA uptake. Except for the competence protein these proteins bind two zinc ions per molecule as cofactor.

The human gene was searched against the Protein Data Bank's 3D structural database using BLASTP to identify possible structures against which GLOL may be modeled. The PDB ID code entries 1QH5 (human glyoxalase II with S-[N-hydroxy-N-bromophenylcarbamoyl] glutathione) and 1QH3 (human glyoxalase II with cacodylate and acetate ions present in the active site) were shown to be similar enough based on the primary sequence to warrant an effective modeling (25% identical, 43% similar over a stretch of amino acids 175 residues long in the GLOL sequence). The three dimensional representation of the human glyoxlase II is presented in FIG. 11.

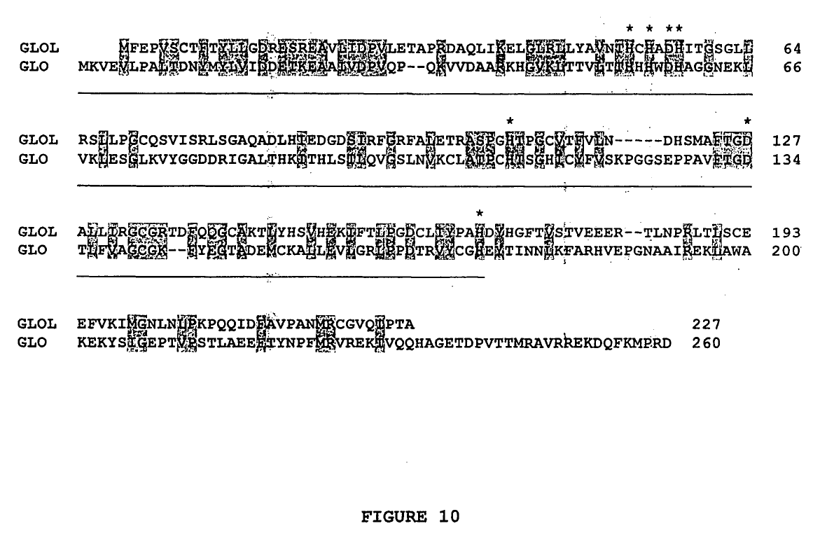

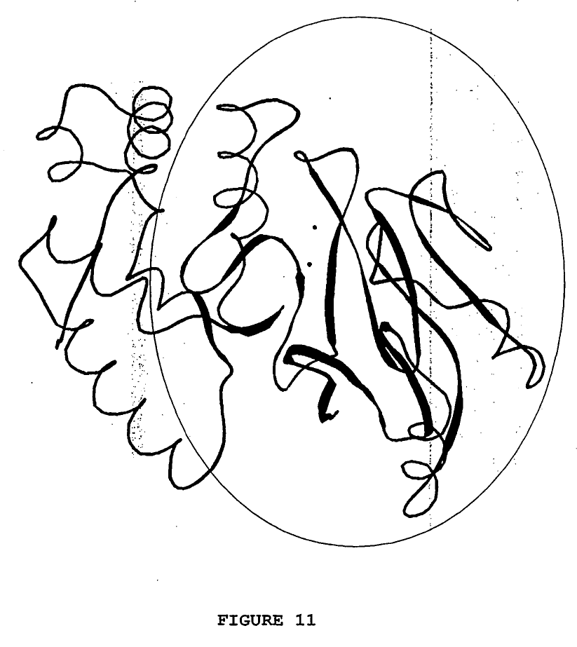

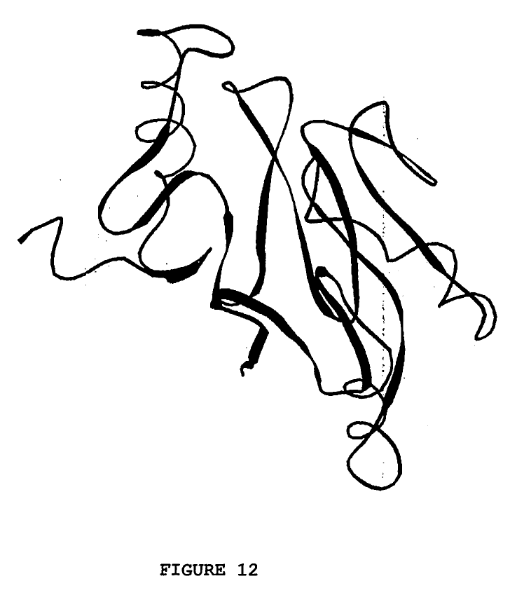

The model of GLOL is for domain one (residue 1-168 in GLOL). The three dimensional representation of the GLOL is presented in FIG. 12. All 7 of the major amino acid residues involved in metal coordination in the human glyoxalase II (Genbank Accession Number NP_{—}005317), listed below, are completely conserved in GLOL in primary structure (FIG. 10) and are highly conserved in tertiary structural space.

| Residue in glyoxalase II | Residue in GLOL |
|---|---|
| H54 | H52 |
| H56 | H54 |
| D58 | D56 |
| H59 | H57 |
| H110 | H108 |
| D134 | D127 |
| H173 | H168 |

Other important residues involved in the binding of glutathione to glyoxalase II or to the formation of the active site (Cameron et al., 1999, Structure Fold. Des. 7: 1067-1078) are listed below along with how these residues correlate to similar residues in GLOL.

| Residue in glyoxalase II | Residue in GLOL |
|---|---|
| D29 | D25 |
| T53 | T51 |
| T111 | T109 |
| G133 | G126 |
| C141 | C134 |
| K143 | R136 |
| E174 | D169 |
| Y175 | Y170 |

These models provide important confirmation of the catalytic activity of GLOL and point to residues that are critical at the active site.

Using the rat partial cDNA clone as the probe for Northern blotting, the relative abundance of mRNA for the GLOL gene in rat liver was dramatically decreased after PUFA treatment (see Tables 1 and 2).

Northern blot studies evaluating tissue distribution in rat showed that GLOL transcript is expressed in all tissues examined, with high levels in liver and kidney. Similar studies in human also showed ubiquitous expression with high levels in liver, kidney and skeletal muscle and highest levels in colon.

These observations confirm that GLOL is a ubiquitously expressed glyoxalase II-like protein regulated by polyunsaturated fatty acids. Thus, the present invention describes a novel drug target, i.e. GLOL, which includes the use of the gene as well as the promoter to screen for novel drugs or chemical entities that modulate expression of the gene or activity of the gene product.

III FTF1 Gene and Polypeptide

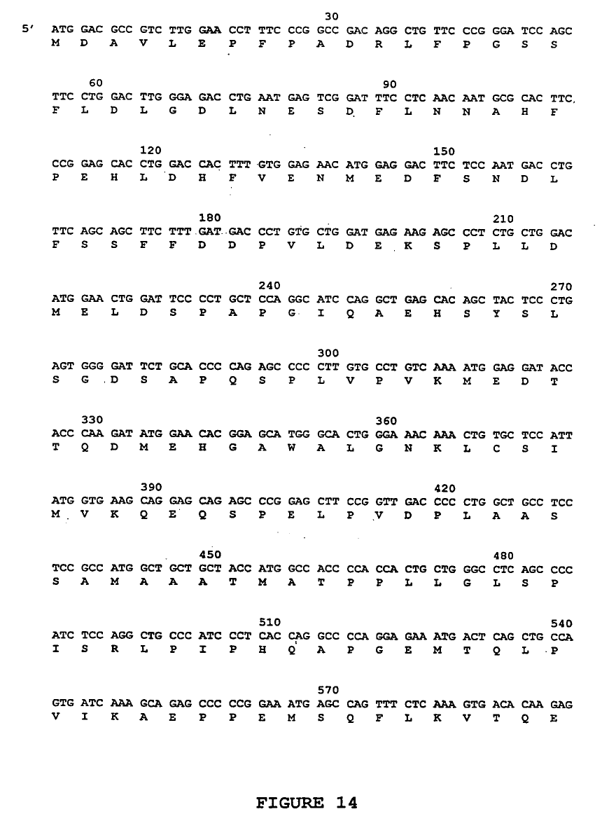

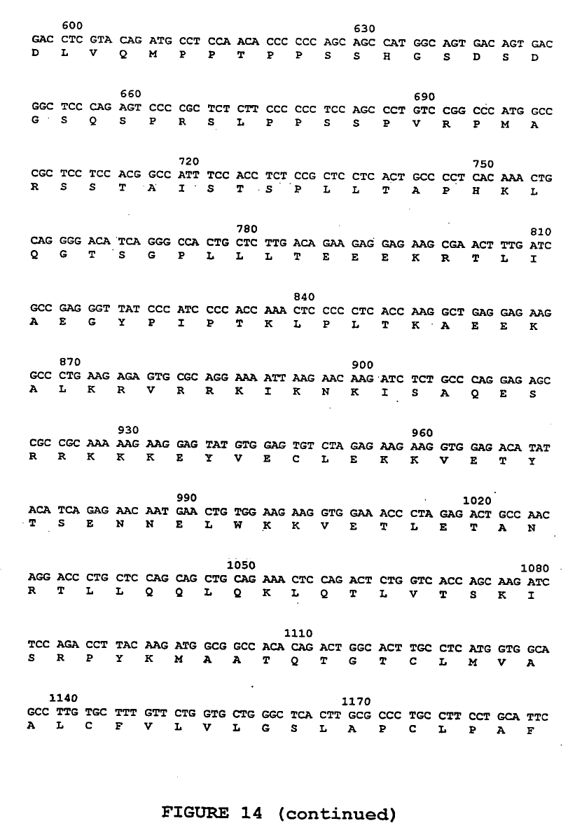

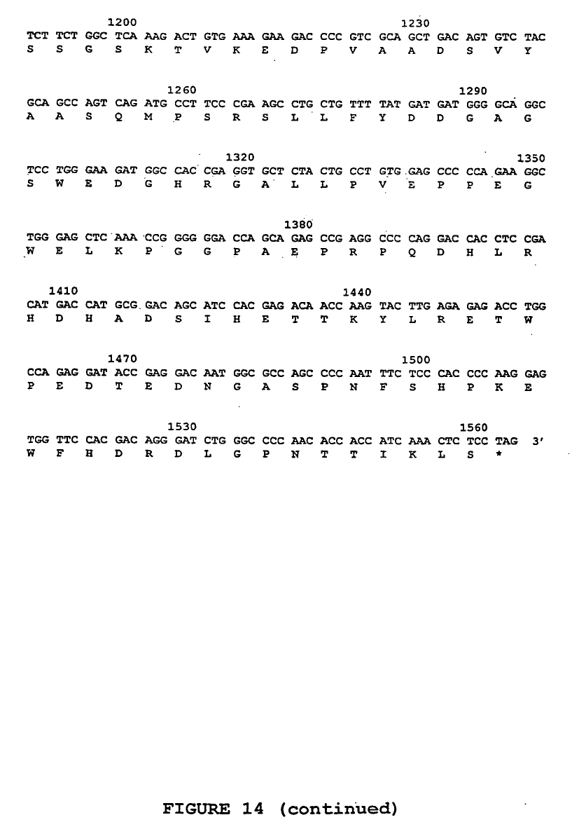

Another of the differentially expressed bands identified during the study was a rat partial cDNA clone orthologous to the mouse OASIS gene. Using 5′-rapid amplification of cDNA ends (RACE), the 5′ end of the rat FTF1 (fat responsive transcription factor 1) gene was cloned and the full length coding sequence (CDS) determined by DNA sequencing (FIG. 14).

Honma et al. (1999, Mol. Brain Res., 69: 93-103) have identified and characterized the mouse OASIS gene (old-astrocyte specifically-induced substance). They indicate that the gene encodes a transcription factor belonging to the CREB/ATF gene family and have shown that it contains a leucine zipper motif and basic DNA-binding domain. These investigators showed that expression of the gene was induced upon cryo-injury to the mouse brain. They did not indicate any association of this transcription factor with PUFAs or with diabetes.

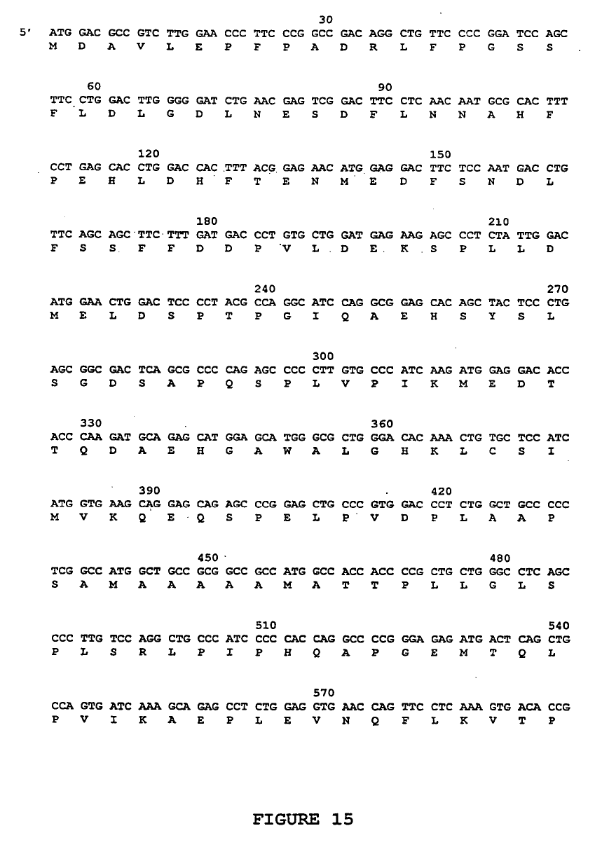

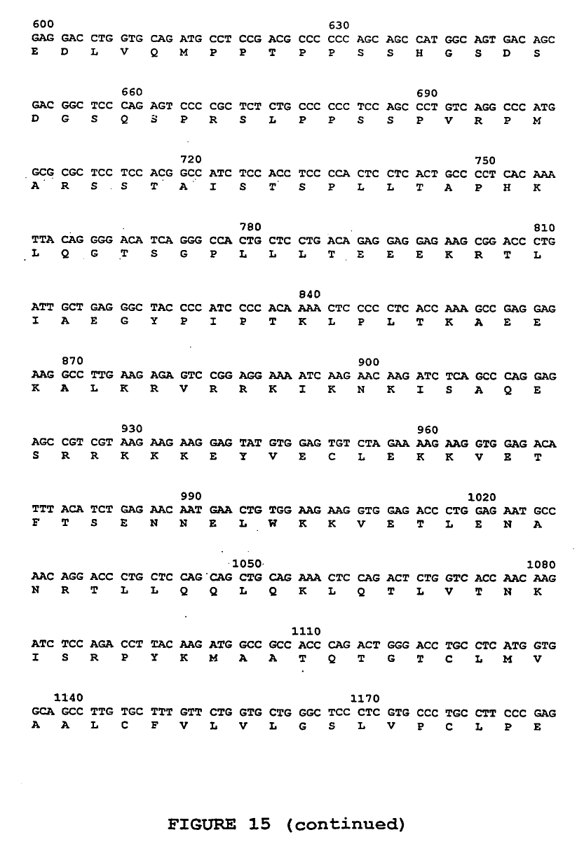

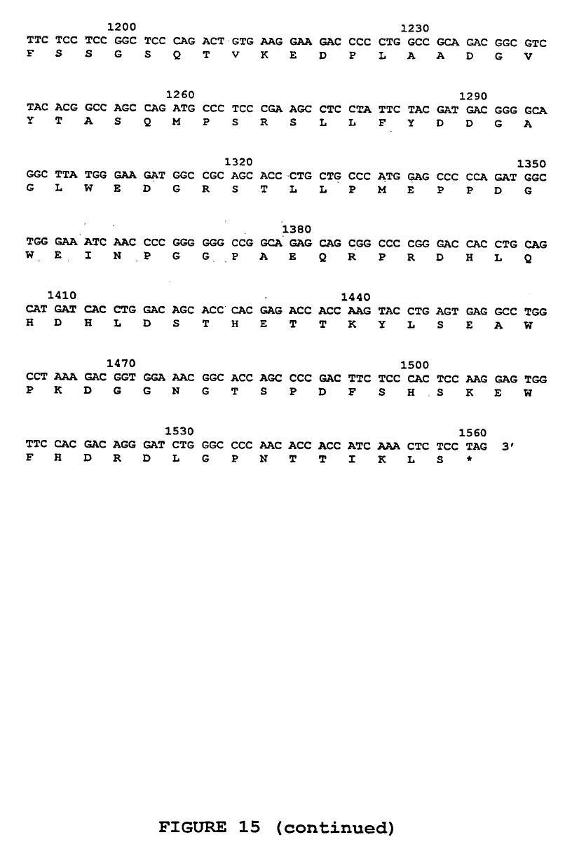

The human gene was identified by searching GenBank's HTGS database with the mouse OASIS amino acid sequence using TBLASTN and the mouse OASIS cDNA sequence using BLASTN. Exons were mapped onto this sequence (GenBank Accession No. AC009647) of genomic DNA using standard bioinformatics methods and the gene was identified as being composed of 12 exons. The human gene was cloned using primers designed using the sequence for hFTF1 derived from the GenBank HTGS database and the full length CDS was sequenced (FIG. 15).

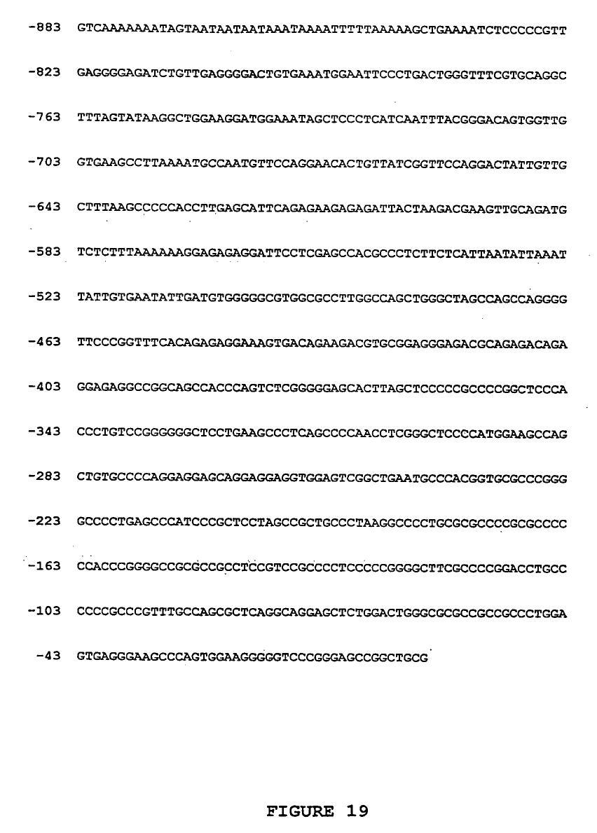

Using bioinformatic techniques, the present inventors identified and mapped out the control region of the human FIF1 gene. By searching GenBank's EST division, they identified a collection of human ESTs containing 5′ UTR for the gene and identified an area containing highly repetitive elements upstream from the -883 position. The control region between position-1 and -883 from the ATG is shown in FIG. 19.

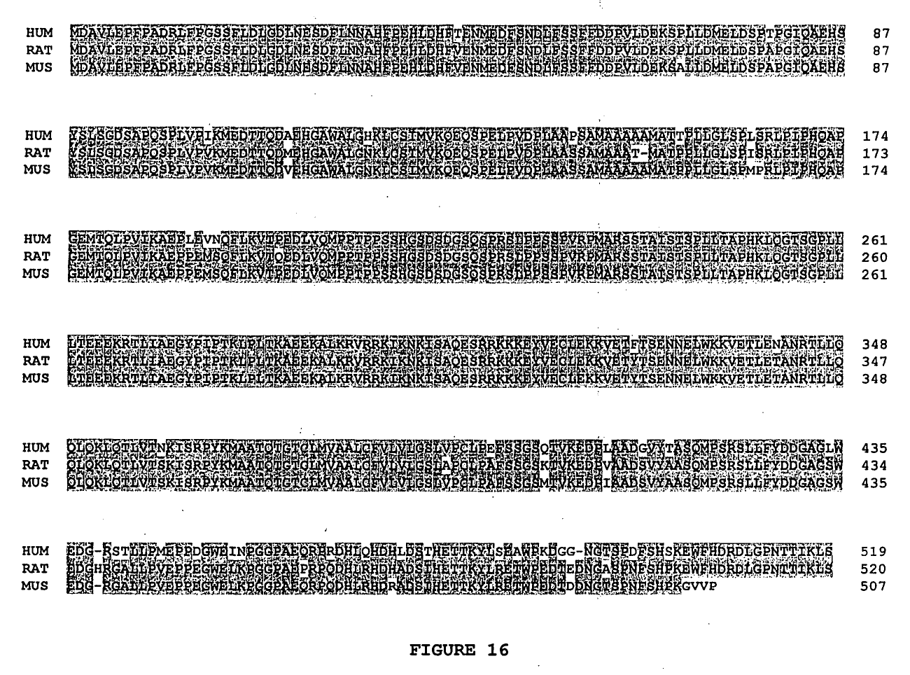

A multiple sequence alignment indicating the similarities among the human and rat FTF1 genes and the mouse OASIS gene is presented in FIG. 16. The human and rat FTF1 genes are 91% identical or 93% similar. The human and mouse genes are 90% identical while the rat and mouse genes are 95% identical.

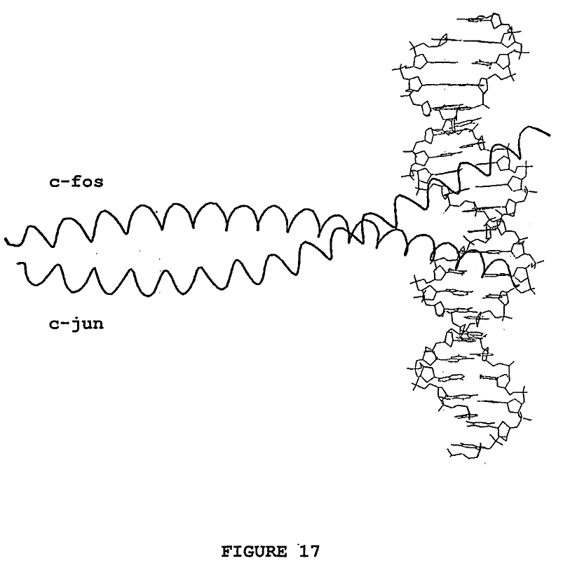

GenBank's PDB database containing three-dimensional structures was searched using BLASTP to identify possible structures against which FTF1 may be modeled. A portion of the structure of the transcription factor c-jun (PDB ID code: 1FOS, c-jun proto-oncogene [transcription factor ap-1] dimerized with c-fos and complexed with DNA) was identified as the template structure (FIG. 17). FTF1 was 37% identical to c-jun over a 43 amino acid stretch of sequence.

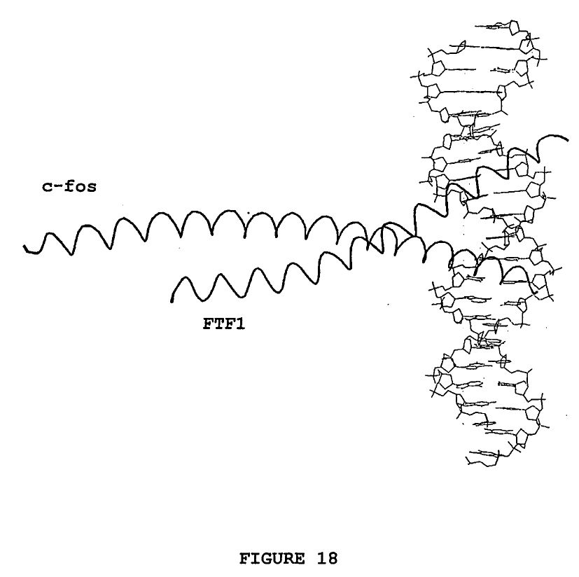

Using comparative modeling, amino acid residue positions 295-337 were modeled onto the structure of c-jun. This corresponds to the basic DNA binding region as well as the leucine zipper motif (FIG. 18).

The human FTF1 gene contains the “bZIP transcription factors basic domain signature” (amino acids 295-310) and the “leucine zipper pattern” motifs (amino acids 332-353) as assessed by PROSITE. These motifs fall within the coiled coil prediction region (amino acids 285-359). The 3D-modeled region contains the bZIP basic domain signature and the N-terminal end of the beginning of the leucine zipper towards the C-terminus.

Residues 285-359 were mapped with high certainty as containing an amino acid sequence highly favored to form a coiled coil structure using the COILS v2.1 software (Lupas et al., 1991, Science, 252: 1162-1164). This coiled coil structure can be seen in the 3D model presented in FIG. 18.

Using the rat partial cDNA clone as the probe for Northern blotting, the relative abundance of mRNA for the FTF1 gene in rat liver was dramatically decreased after PUFA treatment (Tables 1 and 2).

Northern blot studies evaluating tissue distribution in rat showed that FTF1 transcript is expressed in all tissues examined, with highest levels in adipose, ovaries and nerve. Similar studies in human showed high levels of expression in colon, placenta, lung, small intestine and heart.

These observations confirm that FTF1 is a widely expressed transcription factor regulated by polyunsaturated fatty acids. Thus, the present invention describes a novel drug target, i.e. FTF1, which includes the use of the gene and/or the use of the promoter to screen for novel drugs or chemical entities that modulate expression of fat regulated transcription factors.

IV. A1BG Gene and Polypeptide

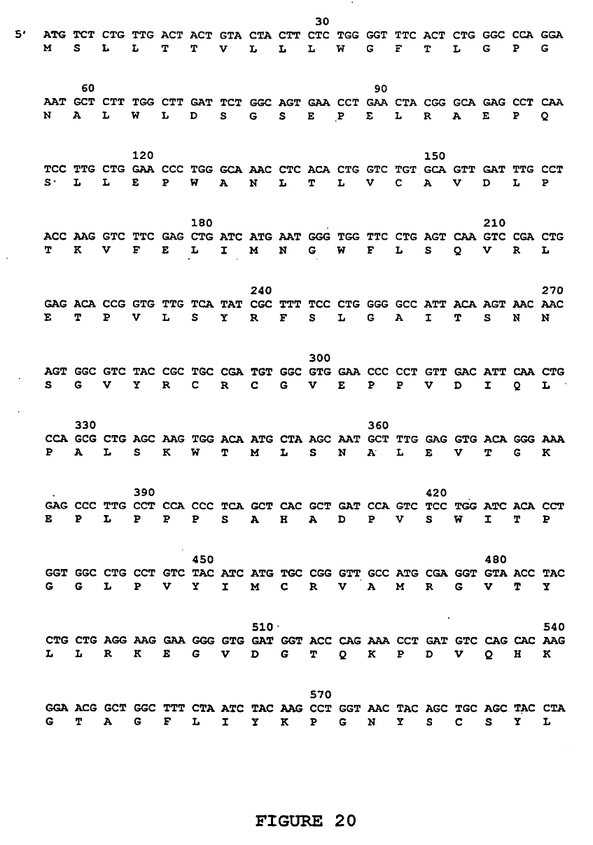

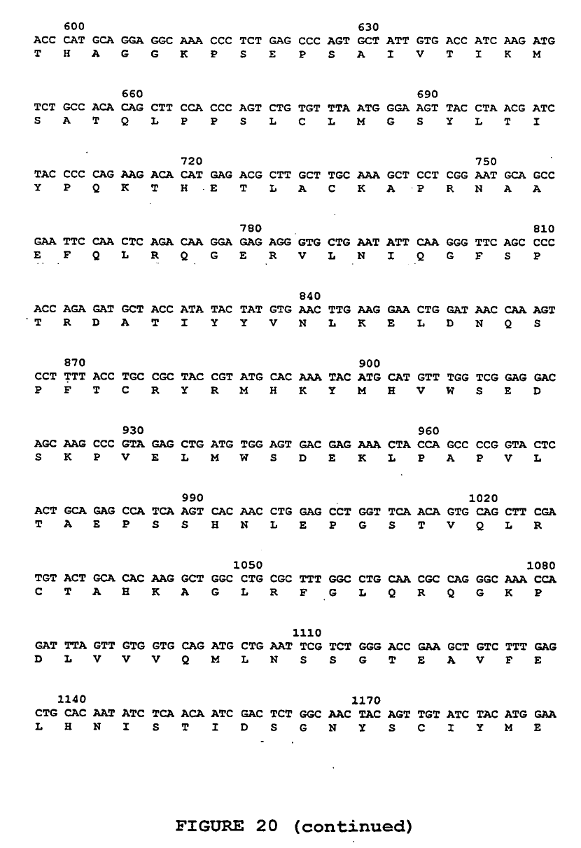

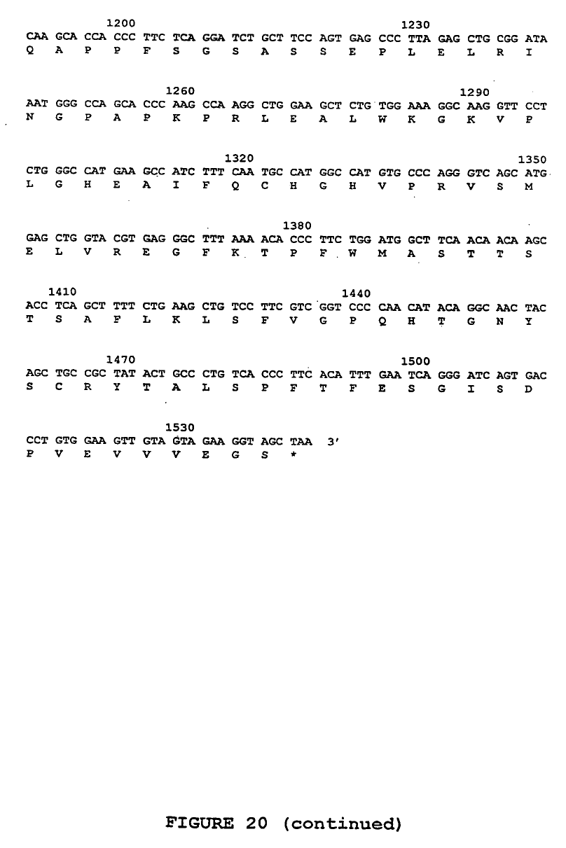

Another of the differentially expressed bands identified during the study was a rat partial cDNA clone highly similar to human alpha-1b-glycoprotein (A1BG). Using 5′-rapid amplification of cDNA ends (RACE), the 5′ end of the rat A1BG was cloned and the full length coding sequence (CDS) was determined by DNA sequencing (FIG. 20).

The mature human A1BG protein (474 amino acid residues) has been previously purified and the amino acid sequence of the protein determined by conventional protein sequencing (Ishioka et al., 1986, Proc. Natl. Acad. Sci., 83: 2363-2367) but the gene has never been cloned or characterized.

The sequenced protein for A1BG did not begin with a methionine. Since A1BG is a plasma protein, it was hypothesized that the nascent protein should contain a signal sequence for the secretory pathway. Using a TBLASTN query with the human protein sequence in GenBank's EST division (dbest), the present inventors discovered a human EST (W25099) for the 5′ end of the A1BG gene, which contained the missing exons for the beginning of the transcript. This EST extended the knowledge of the 5═ end of the gene to include 15 nucleotides of 5′ UTR in exon 1 and most of exon 2 (see below).

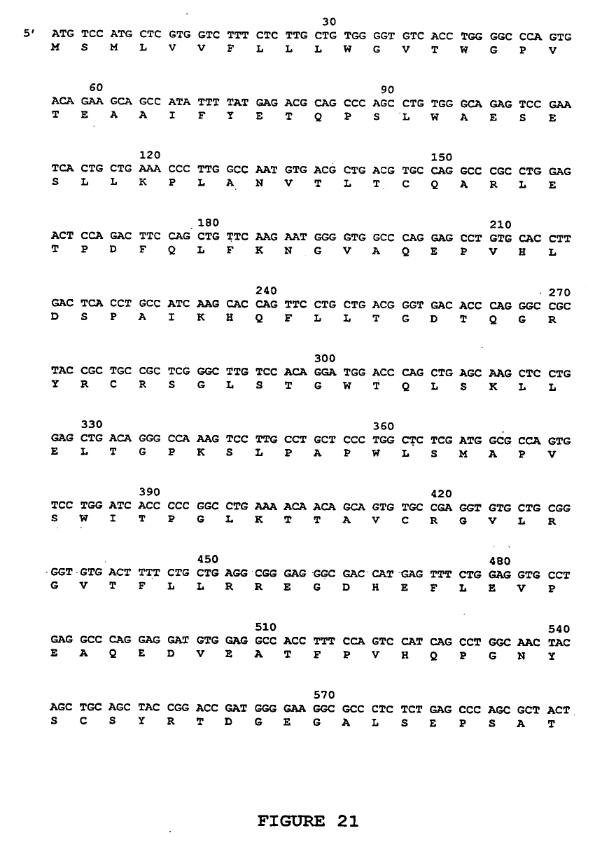

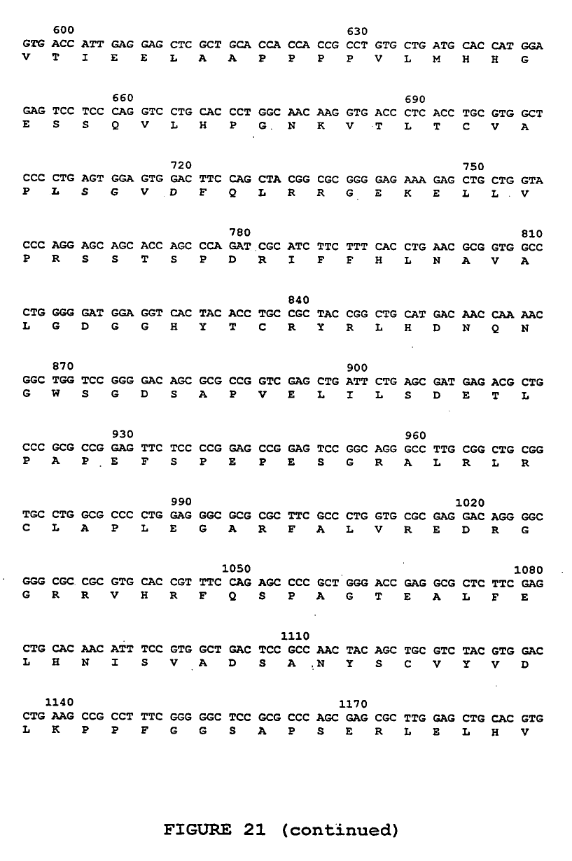

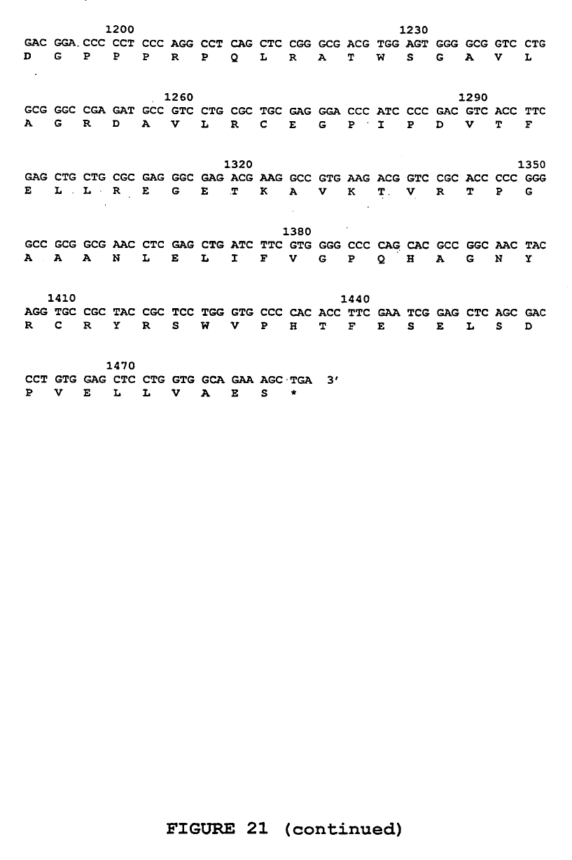

Using bioinformatic techniques, the human A1BG gene was located in the GenBank HTGS database on a fragment of genomic DNA from chromosome 19 using a TBLASTN query with the human protein sequence (GenBank Accession No. AC012313). Exons were mapped onto this sequence showing that the gene was constructed of 8 exons. Oligonucleotide primers were synthesized and the human gene sequence was verified by cloning and DNA sequencing. The human A1BG gene sequence is presented in FIG. 21.

The amino acid sequence in this newly discovered N-terminus contains 21 residues that conform well to classic signal sequences with a predicted cleavage site that generates the N-terminus of the mature protein. Both a neural network and a hidden Markov model predicted the same cleavage site with high probability (Henrik et al., 1997, Prot. Eng., 10: 1-6). Therefore, the nascent protein contains 495 amino acid residues rather than 474 residues as previously reported.

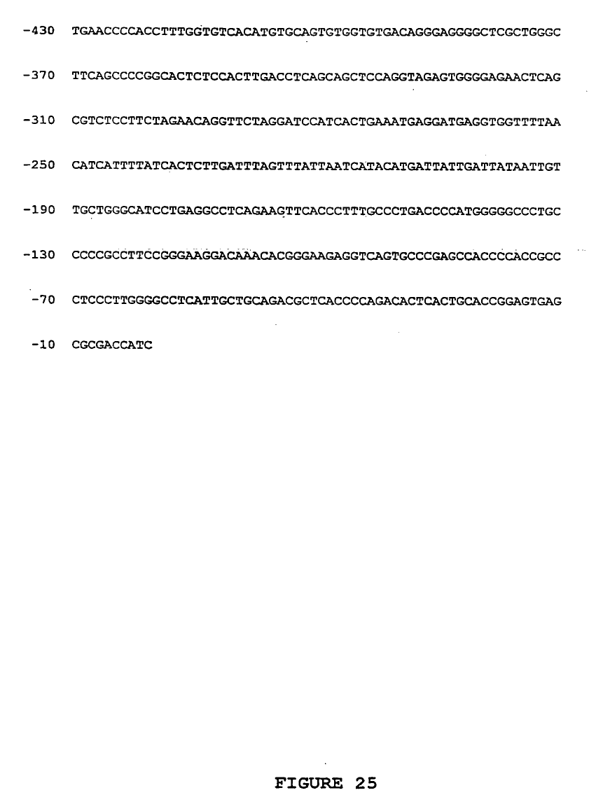

Using bioinformatic techniques, the present inventors identified and mapped out the control region of the human A1BG gene. As previously indicated, they had identified a human EST (GenBank Accession No. W25099) containing 15 bp of 5′ UTR. Searching GenBank's EST division, they further identified a transcriptionally active region upstream from the -430 position. The control region between positions-1 and -430 from the ATG is shown in FIG. 25.

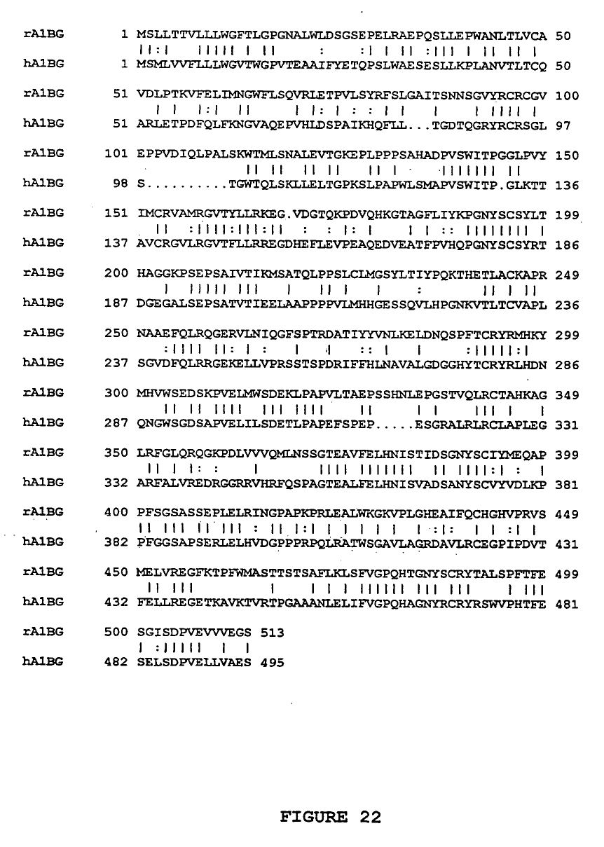

The rat and human A1BG were compared and found to be 44% identical or 55% similar at the amino acid sequence level (FIG. 22).

A1BG belongs to the immunoglobin superfamily of proteins with signature motifs for the immunoglobin and major histocompatibility complex (MHC) domains. A1BG shows a pentapartite domain structure. Each domain contains a disulfide bridge. The human protein is glycosylated at 4 asparagines throughout the sequence and contains a glucosamine attachment site.

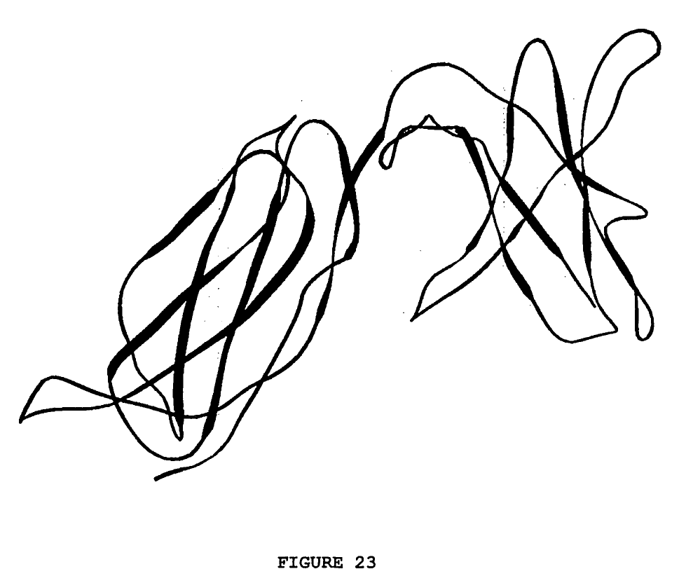

The human A1BG was searched against the pdb database to identify proteins with known structures that were similar enough for an effective modeling. A number of natural killer (NK) cell inhibitory receptors (KIR) structures were identified as good candidates. KIR2DL2 was chosen as the appropriate structure for comparative modeling (PDB ID code: 2DLI—Killer Immunoglobulin Receptor 2dl2, Trigonal Form). See FIG. 23.

NK cells activate their cytolytic killing against certain pathogen-infected or tumor cells with a concomitant discrimination between self and nonself, thereby directing the NK-mediated lysis only against appropriate target cells. One mechanism to achieve self-recognition is through regulation by cell surface inhibitory receptors. These receptors are capable of interacting with class I MHC molecules expressed on the target cell surface, abnormal cells being deficient in class I major histocompatibility complex molecules.

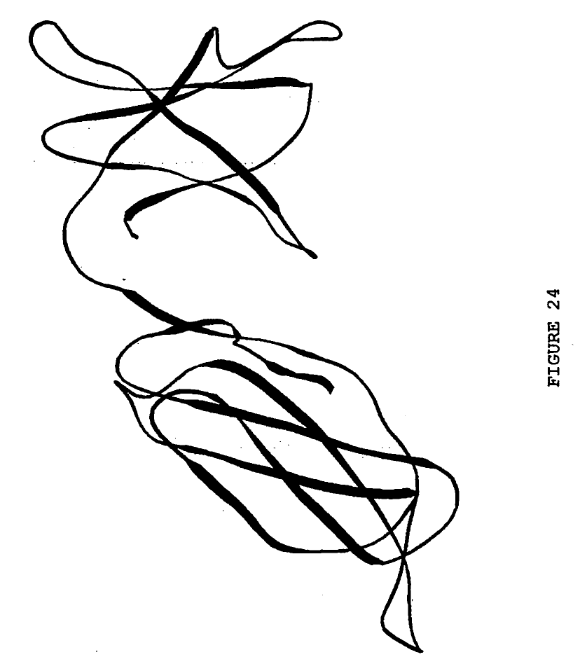

As shown in FIG. 24, the last two domains of A1BG were modeled using this template structure due to the fact that the extracellular MHC class I-binding portion of KIR2DL2 is made up of only 2 domains.

Partial amino acid sequencing of antihemorrhagic factors from opossum (Catanese J. J. and Kress L. F., 1992, Biochemistry, 31: 410-418) and mongoose (Qi et al., 1994, Toxicon, 32: 1459-1469 and Qi et al., 1995, Toxicon, 33: 241-245) show a high degree of similarity to A1BG. These are plasma glycoprotein AHF1/AHF2 from mongoose (antihemorrhagic factor against snake venom metalloproteinases) and plasma glycoprotein oprin from opossum (proteinase inhibitor active against snake venom metalloproteinases). The known sequence of AHF1/AHF2 is approximately 50% identical to A1BG while the known sequence of oprin is approximately 35% identical, strongly suggesting that these proteins are, in fact, the orthologs to the human A1BG. It should be noted, however, that the human A1BG is reported not to have antihemorrhagic activity (Ishioka et al., 1986, Proc. Natl. Acad. Sci., 83: 2363-2367).

The present inventors' data are consistent with other reports and publications on A1BG, however, no other investigators have identified a role for this protein. The present inventors have observed that alpha-1b-glycoprotein is similar to the natural killer cell inhibitory receptor structures, which are important in controlling immune system functioning.

Using the rat partial cDNA clone as the probe for Northern blotting, the relative abundance of mRNA for the A1BG gene in rat liver was increased after PUFA supplementation (see Tables 1 and 2). The increased expression of A1BG mRNA was quite varied among the individual supplemented fatty acids with the highest expression observed in the DHA-treated group.

Northern blot studies evaluating tissue distribution in human and rat showed that A1BG transcript is highly liver specific.

These observations confirm that A1BG is a plasma protein produced by the liver and regulated by polyunsaturated fatty acids. The present inventors' data suggest that mRNA abundance is decreased in STZ-induced diabetic rats. Thus, the present invention describes a novel drug target, i.e. A1BG, which includes the use of the gene as well as the promoter to screen for novel drugs or chemical entities that modulate expression of the gene or activity of the gene product, and for new test compounds effective against immunological diseases.

V. SCD Genes and Polypeptides

Two more differentially expressed bands identified during the study were rat partial cDNA clones for rat stearoyl-CoA desaturases (SCD1 and SCD2, GenBank Accession Nos. J02585 and AB032243, respectively) with known metabolic function. Furthermore, work from the present inventors' laboratory shows that mRNA expression as well as SCD activity is reduced in streptozotocin (STZ)-induced diabetes. This is also true for SCD1 mRNA expression in diabetic mice (Waters K. M. and Ntambi J. M., 1994, J. Biol. Chem., 269: 27773-27777). Conversely, the human SCD mRNA expression is high in some cancerous tissues (Li et al., 1994, Int. J. Cancer, 57: 348-352). These data suggest that the rat SCD1 and SCD2 genes, as well as human SCD gene, are transcriptionally regulated by dietary fat (especially PUFAs, see Table 2) and in disease (Ntambi J. M., 1999, J. Lipid Res., 40: 1549-1558).

The mouse and rat proteins have also been characterized and the function established (Miyazaki et al., 2000, J. Biol. Chem., 275: 30132-30138 and Thiede et al., 1986, J. Biol. Chem., 261: 1323-1325). SCD1 is proposed to desaturate either palmitoyl-CoA (16:0) or stearoyl-CoA (18:0), while SDC2 is proposed to exhibit selectivity for desaturases of 18:0 relative to 16:0 (Kim et al., 2000, J. Lipid Res., 41: 1310-1316). The functional analysis of the human SCD protein has not been reported (Zhang et al., 1999, Biochem. J., 340: 252-264).

Polyclonal antibodies against rat SCD have been produced and used in immunoblot analyses (Hassan et al., 2000, Proc. Natl. Acad. Sci., 97: 8883-8888).

In mouse, promoter elements responsible for PUFA repression of SCD1 and SCD2 have been identified (Ntambi J. M., 1999, J. Lipid Res., 40: 1549-1558). A highly conserved region of the two isoform mouse promoters are 77% identical. These elements include sterol regulatory element (SRE), CCAAT-binding factor or nuclear factor (NF-Y) binding site and enhancer binding protein sequences (C/EBP) which are collectively called PUFA-responsive region (PUFA-RR). Despite the clear transcriptional regulation of human SCD in disease, the human SCD control region has not been investigated.

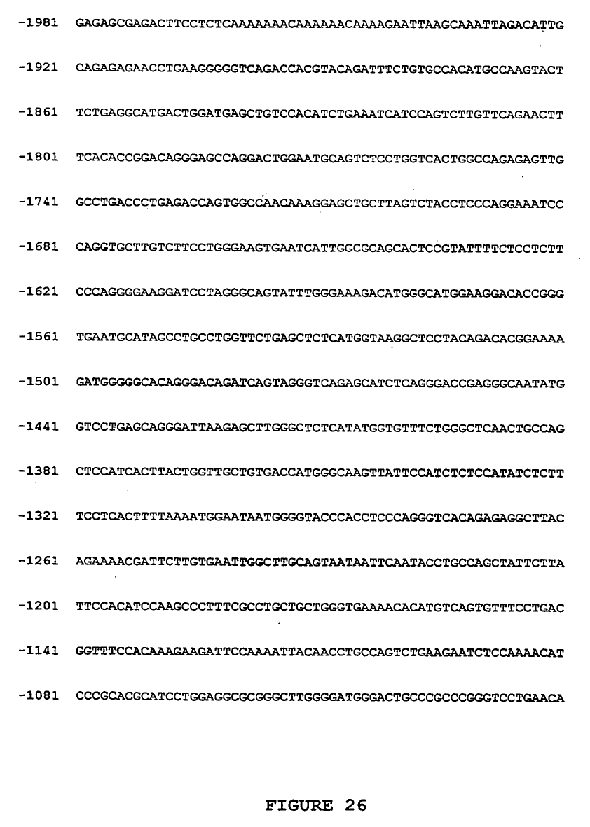

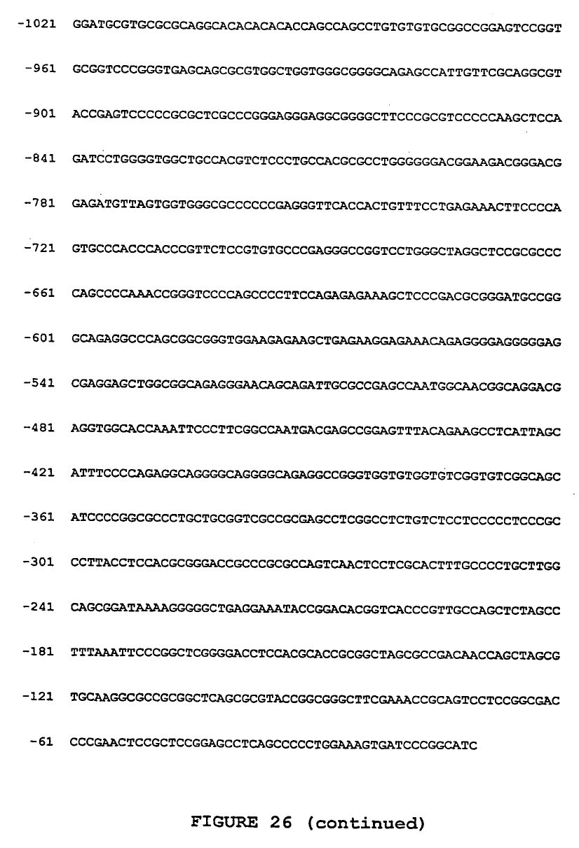

GenBank's HTGS division was searched using BLASTN and the available human SCD cDNA sequence (GenBank Accession Nos. Y13647 and AF097514), to identify human sequences containing the human SCD control region. The search identified a genomic sequence assigned to chromosome 10 (GenBank Accession No. AL139819) containing 6 exons which correspond to the human cDNA. The first exon contains the ATG and 5′ UTR of the human SCD cDNA. The sequence upstream to the ATG from position-1 to -2006 bp was mapped out and a 1970 bp fragment of the sequence was cloned by PCR using specific primers. The nucleotide sequence is shown in FIG. 26.

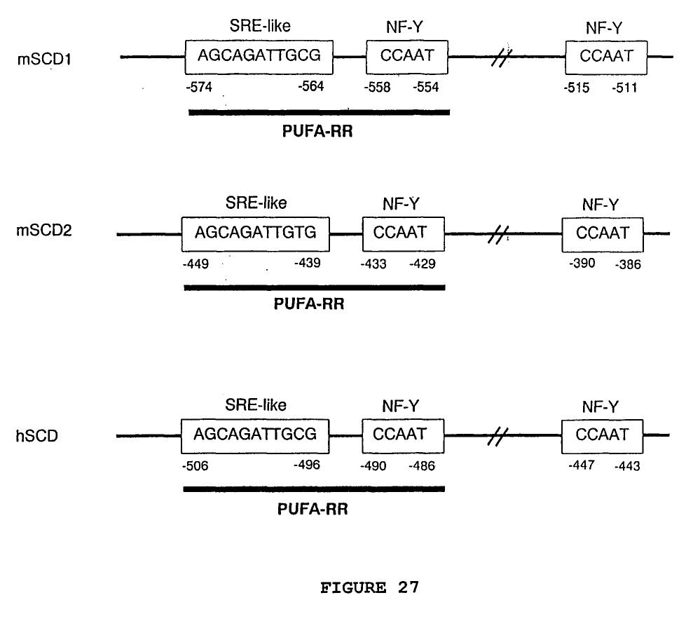

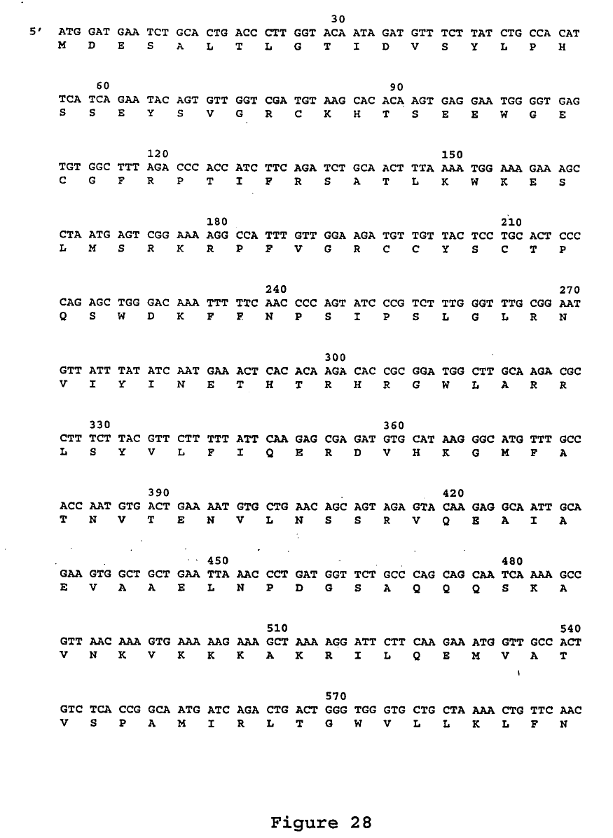

The nucleotide sequence of the human SCD control region was compared with the mouse SCD1 and SCD2 control regions (FIG. 27). The PUFA-RR elements present in the mouse SCD1 and SCD2 promoters are conserved in the human SCD control region. It is likely that these motifs within the control region of the human SCD regulate the expression of the human SCD gene by PUFAs.

Therefore, the present inventors cloned the human control region, which mediates transcription of the SCD gene. As an embodiment of the invention, the present inventors synthesized a human SCD promoter/reporter construct to be used for screening chemical libraries for test compounds which might be useful in the treatment of lipid related diseases.

These observations demonstrate novel methods for identifying target test compounds against a novel drug target, i.e. SCD, which includes the use of the gene as well as the promoter to screen for novel drugs or chemical entities that modulate expression of the gene or activity of the gene product.

VI. Apolipoprotein A-1 Gene and Polypeptide

Another of the differentially expressed bands identified during the study was a rat partial cDNA for apolipoprotein A-1 (ApoA-1). The apolipoprotein A-1 cDNA has been cloned from rat (Haddad et al., 1986, J. Biol. Chem., 261:13268-13277; GenBank Accession No. J02597) and human (Breslow et al., 1982, Proc. Natl. Acad. Sci., 79: 6861-6865; GenBank Accession No. NM_{—}000039).

ApoA-1 is the major protein component of high density lipoprotein (HDL) in the plasma. ApoA-1 is a cofactor for lecithin/cholesterol acyltransferase (LCAT) which is responsible for the formation of most plasma cholesteryl esters (Soutar et al., 1975, Biochemistry, 14: 3057-3064). Transfection studies using the mouse and human ApoA-1 promoters have been described (Srivastava et al., 2000, Eur. J. Biochem., 267: 4272-4280). This protein is often measured by radioimmunoassay using antibodies (Karlin et al., 1976, J. Lipid Res., 17: 30-37). The use of combinatorial libraries adapted for monovalent phage display, to isolate proteins (affibodies) that bind selectively to human ApoA-1, has been described (Nord et al., 1997, Nat. Biotechnol., 15: 772-777).

PUFAs have been reported to decrease ApoA-1 mRNA levels (Berthou et al., 1995, Eur. J. Biochem. 232: 179-187) consistent with the results from the present inventors' Northern blot data (see Table 2). The present inventors' data suggest that mRNA abundance is increased in STZ-induced diabetic rats.

These observations demonstrate a novel methods for identifying target test compounds against a novel drug target, i.e. ApoA-1, which includes the use of the gene as well as the promoter to screen for novel drugs or chemical entities that modulate expression of the gene or activity of the gene product.

VII. Delta-3, delta-2-enoyl-CoA Isomerase Gene

Another of the differentially expressed bands identified during the study was a rat partial cDNA for the mitochondrial beta-oxidation auxiliary enzyme delta-3, delta-2-enoyl-CoA isomerase (GenBank Accession No. X61184), involved in the metabolism of PUFAs (Palosaari P. M. and Hiltunen J. K., 1990, J. Biol Chem., 265: 2446-2449 and Muller-Newten G. and Stoffel W., 1991, Biol. Chem. Hoppe-Seyler, 372: 613-624). This enzyme catalyses the conversion of cis-delta-3-enoyl-CoA to trans-delta-2-enoyl-CoA. The rat gene is induced by 3-thia fatty acids (Madsen et al., 1998, J. Lipid Res., 39: 583-593).

The present inventors have shown that mRNA expression is induced in STZ-induced diabetic rats. Several human genetic diseases are associated with genes/enzymes of beta-oxidation, for example, carnitine transport defect, Zellweger syndrome, X-linked adrenoleukodystrophy (Wanders et al., 1992, J. Inherit. Metab. Dis., 15: 643-644; Roe et al., 1990, J. Clin. Invest., 85: 1703-1707 and Wei et al., 2000, Ann. Neurol., 47: 286-296).

The human gene encoding the delta-3, delta-2-enoyl-CoA isomerase has been cloned (Janssen et al., 1994, Genomics, 23: 223-228; GenBank No. NM_{—}001919). The control region of the human gene has been published (Janssen et al., 1994, Genomics, 23: 223-228). The protein has been purified and antibodies raised (Muller-Newten G. and Stoffel W., 1991, Biol. Chem. Hoppe-Seyler, 372: 613-624). The assay for the enzyme activity using substrates like trans-3-hexenoyl-CoA is known by persons skilled in the art (Gurvitz et al., 1999, Biochem. J., 344: 903-914 and Palosaari P. M. and Hiltunen J. K., 1990, J. Biol. Chem. 265: 2446-2449).

Using the rat partial cDNA clone as the probe for Northern blotting the present inventors show that PUFAs increase delta-3, delta-2-enoyl-CoA isomerase mRNA levels (see Tables 1 and 2).

These observations demonstrate novel methods for identifying target test compounds against a drug target, i.e. delta-3, delta-2-enoyl-CoA isomerase, which includes the use of rat and human genes as well as promoters to screen for novel drugs or chemical entities that modulate expression of the gene or activity of the gene product.

VIII. Fatty Acid Synthase Gene and Polypeptide

One of the differentially expressed bands was identified as fatty acid synthase (FAS). FAS cDNAs have been characterized from several species including rat (GenBank Accession Nos. X6288S and M76767) and human (GenBank Accession Nos. U26644 and U29344).

FAS catalyzes the synthesis of the long chain fatty acid palmitate from acetyl-CoA, malonyl-CoA and NADPH (Wakil S. J., 1989, Biochemistry, 28: 4523-4530). It exists as a homodimer with each peptide subunit about 260 kD in size. The subunit carries seven distinct component activities (beta-ketoacyl synthase; acetyl-CoA and malonyl-CoA transacylases; beta-hydroxyacyl dehydratase; enoyl reductase; beta-ketoacyl reductase; thioesterase) and a site for the prosthetic group 4′-phosphopantetheine (acyl carrier protein). Human FAS purified from recombinant E. coli exhibits all of these activities (Jayakumar et al., 1996, Proc. Natl. Acad. Sci., 93: 14509-14514). Jayakumar et al. (1995, Proc. Natl. Acad. Sci., 92: 8695-8699) purified FAS to near homogeneity from a human hepatoma cell line, HepG2.

The expression of FAS is under nutritional and hormonal regulation (Semenkovich C. F., 1997, Prog. Lipid Res., 36: 43-53). Using the rat partial cDNA clone as the probe for Northern blotting the present inventors' data (Tables 1 and 2) are consistent with data reported previously showing that PUFAs decrease FAS mRNA levels (Clarke et al., 1990, J. Nutr., 120: 225-231 and Armstrong et al., 1991, Biochem. Biophys. Res. Commun., 177: 1056-1061). Moreover, its expression is altered in streptozotocin-induced diabetes (Saggerson E. D. and Carpenter C. A., 1987, Biochem. J., 243: 289-292), which is consistent with the present inventors' results that suggest mRNA abundance is reduced in STZ-induced diabetic rats.

The rat promoter for FAS has been well characterized in both cultured cells and transgenic mice using reporter assay systems (Semenkovich C. F., 1997, Prog. Lipid Res., 36: 43-53). Two promoters have been described for the human FAS gene, one very similar to the rat gene and another, promoter II consisting of ˜260 base pairs of the 3′ terminus of the first intron (Hsu et al., 1996, J. Biol. Chem., 271: 13584-13592).

Antibodies against human FAS are available and have been used for immunohistochemistry and Western blotting (Joyex et al., 1990, J. Clin. Endocrinol. Metab., 70: 1438-1444; Jayakumar et al., 1996, Proc. Natl. Acad. Sci., 93: 14509-14514 and Wilentz et al., 2000, Pediatr. Dev. Pathol., 3: 525-531).

FAS has been proposed as a potential selective target for antineoplastic therapy (Kuhajda et al., 1994, Proc. Natl. Acad. Sci., 91: 6379-6389). A drug screening assay has been described for the FAS protein (Patent Cooperation Treaty International Patent Application No. WO 00/51430).

The present inventors teach novel methods for identifying target test compounds which includes the use of human promoters to screen for novel drugs or chemical entities that modulate expression of the FAS gene.

IX. Glucose-6-Phosphate Dehydrogenase Gene and Polypeptide

One of the differentially expressed bands identified during the study was a rat partial cDNA for glucose-6-phosphate dehydrogenase (G6PD) gene. G6PD cDNAs have been cloned from rat (GenBank Accession No. X07467) and human (GenBank Accession No. X03674).

G6PD catalyzes the first reaction in the pentose phosphate pathway leading to the production of pentose phosphates and reducing power in the form of NADPH for reductive biosynthesis and maintenance of the redox state of the cell.

Human G6PD has been purified from recombinant E. coli (Bautista et al., 1992, Biochim. Biophys. Acta, 1119: 512-518) and from various other sources including human erythrocytes (Adediran S. A., 1996, Biochimie, 78: 165-170) and rat brain (Askar et al., 1996, Indian J. Biochem. Biophys., 33: 512-518

G6PD is subject to tissue-specific regulation by hormones, nutrients and oxidant stress (Kletzien et al., 1994, FASEB J., 8: 174-181). PUFAs have been reported to decrease G6PD mRNA levels (Tomlinson et al., 1998, J. Nutr., 118: 408-415 and Yoshida et al., 1999, J. Nutr. Sci. Vitaminol., 45: 411-421) consistent with the results from the present inventors' Northern blot findings (see Table 2).

Promoters for both rat and human G6PD have been cloned and characterized using reporter assay systems (Ursini et al., 1990, Biochem. Biophys. Res. Commun., 170: 1203-1209; Kletzien et al., 1994, FASEB J., 8: 174-181 and Phillippe et al., 1994, Eur. J. Biochem., 226: 377-384). Antibodies against G6PD are available (Dao et al., 1982, Proc. Natl. Acad. Sci., 79: 2860-2864 and Moore et al., 1986, Carcinogenesis, 7: 1419-1424).

The present inventors teach novel methods for identifying target test compounds for G6PD, which includes the use of rat and human genes as well as promoters to screen for novel drugs or chemical entities that modulate expression of the gene or activity of the gene product.

X. Glycerol-3-Phosphate Acyltransferase Gene and Polypeptide

A mitochondrial and a microsomal isoform of GPAT have been described. One of the differentially expressed bands identified during the study was a rat partial cDNA for the mitochondrial isoform of the glycerol-3-phosphate acyltransferase (GPAT) gene. Only the rat and the mouse cDNAs for the mitochondrial isoenzyme have been cloned (Ganesh et al., 1999, Biochem. Biophys. Acta, 1439: 415-423, GenBank Accession No. AF021348 and Shin et al., 1991, J. Biol. Chem., 266: 23834-23839, GenBank Accession No. NM_{—}008149).

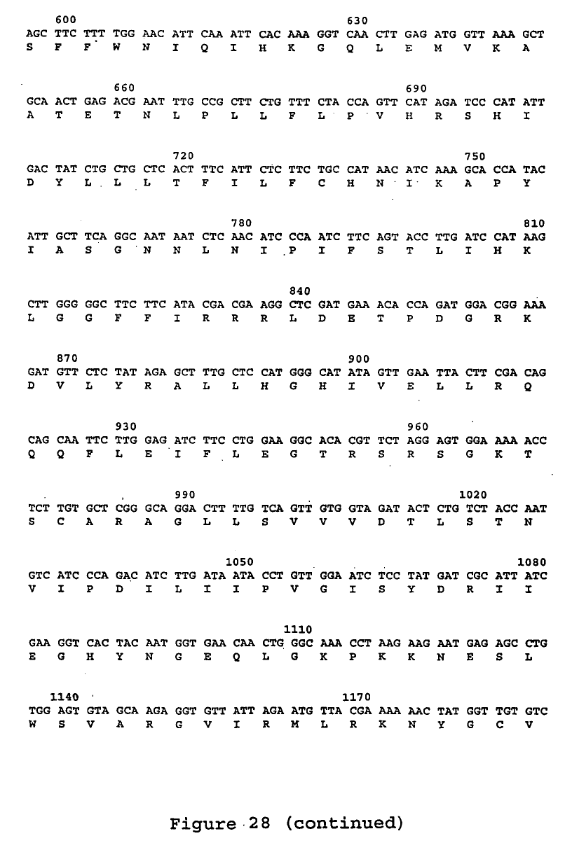

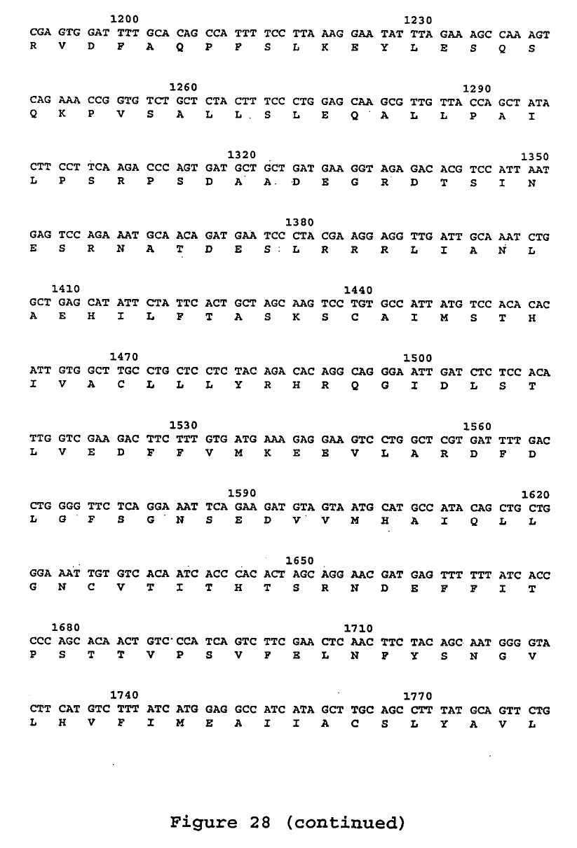

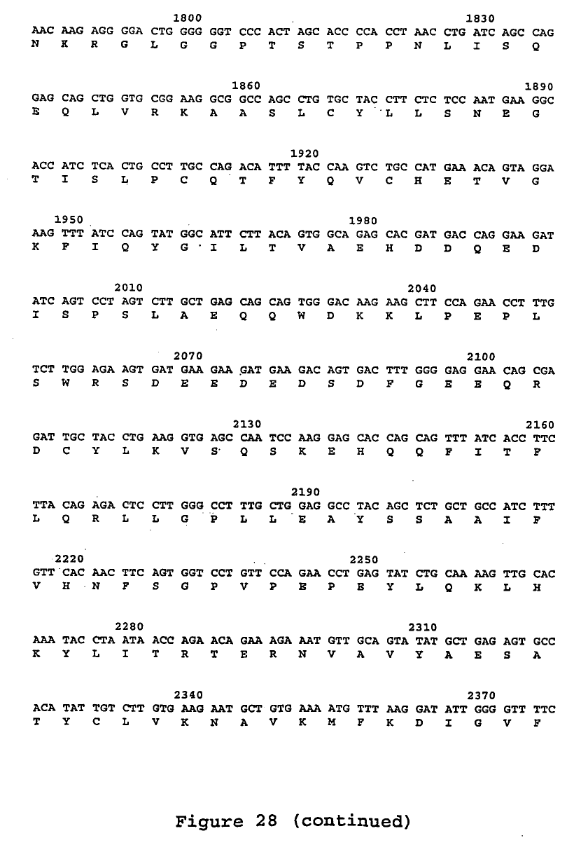

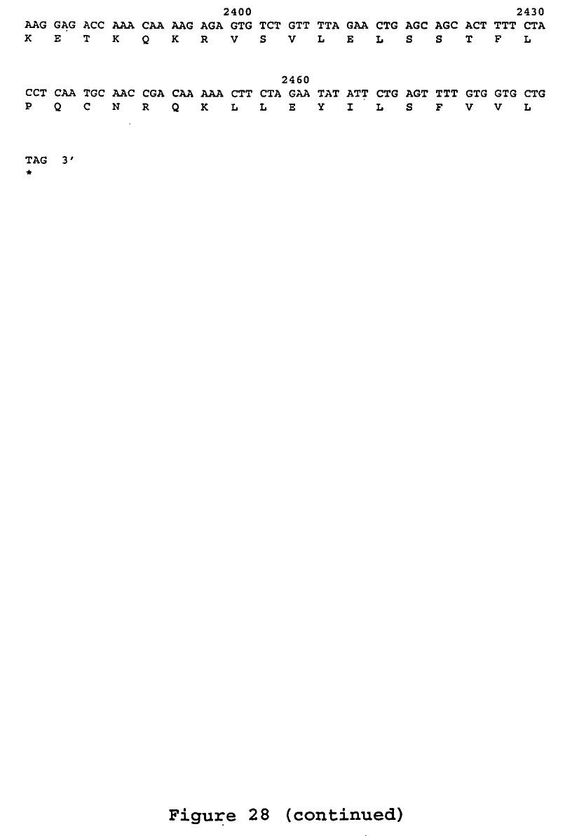

Using bioinformatic techniques, the human gene was located in GenBank's HTGS database on a fragment of genomic DNA from chromosome 10 (GenBank Accession No. AL391986). Oligonucleotide primers were synthesized and the proposed human gene sequence was verified by cloning and DNA sequencing (FIG. 28). Exons were mapped onto genomic DNA from AL135838 and the gene was found to comprise 20 coding exons. Nagase et al. (2000, DNA Res., 7: 273-281) have cloned an incomplete cDNA containing only the carboxyl end of this human GPAT CDS (GenBank Accession No. AB046780). Although the CDS for this human gene sequence has been submitted to GenBank there are no annotations thereon which would suggest its functional identity as the glycerol-3-phosphate acyltransferase gene. It is merely identified as a “mRNA for KIAA1560 protein”.

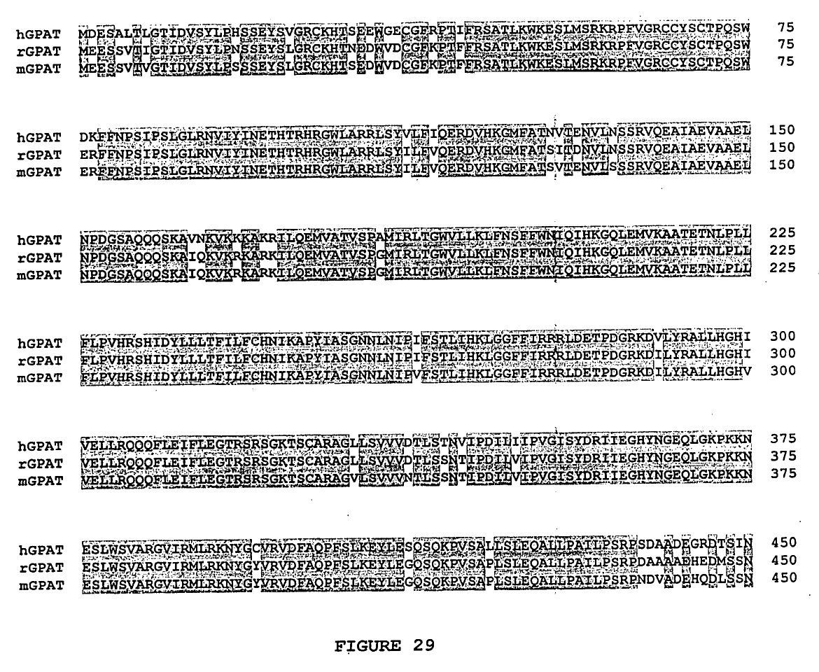

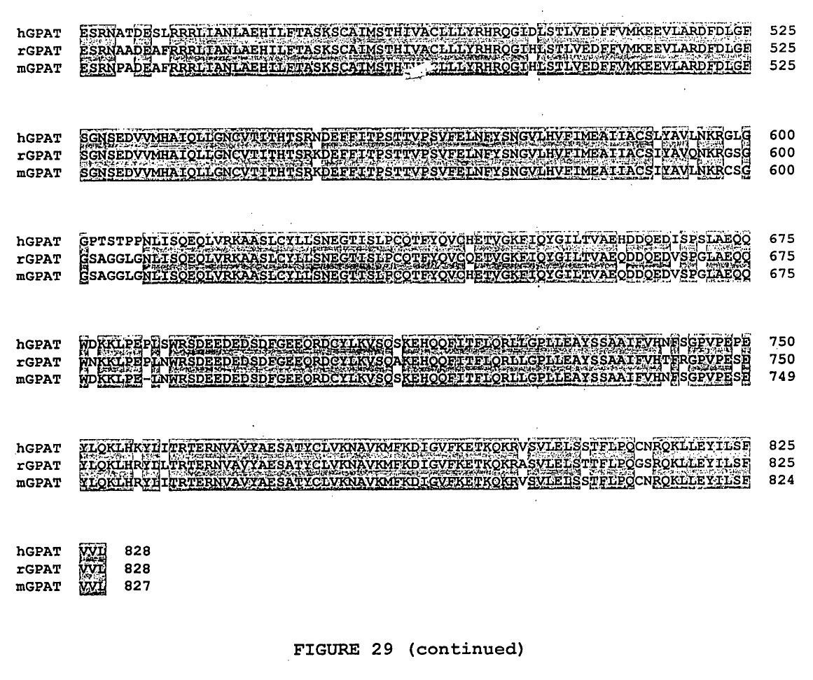

A multiple sequence alignment indicating the identities and similarities among the human, rat (Genbank Accession Number AAB71605) and mouse (Genbank Accession Number NP_{—}032175) GPAT genes is presented in FIG. 29. The human and mouse genes are 93% identical or 96% similar while the human and rat genes are 92% identical or 95% similar.

To date, only the characterization of the mouse promoter has been described (Jerkins et al., 1995, J. Biol. Chem., 270: 1416-1421 and Ericsson et al.,.1997, J. Biol. Chem., 272: 7298-7305).

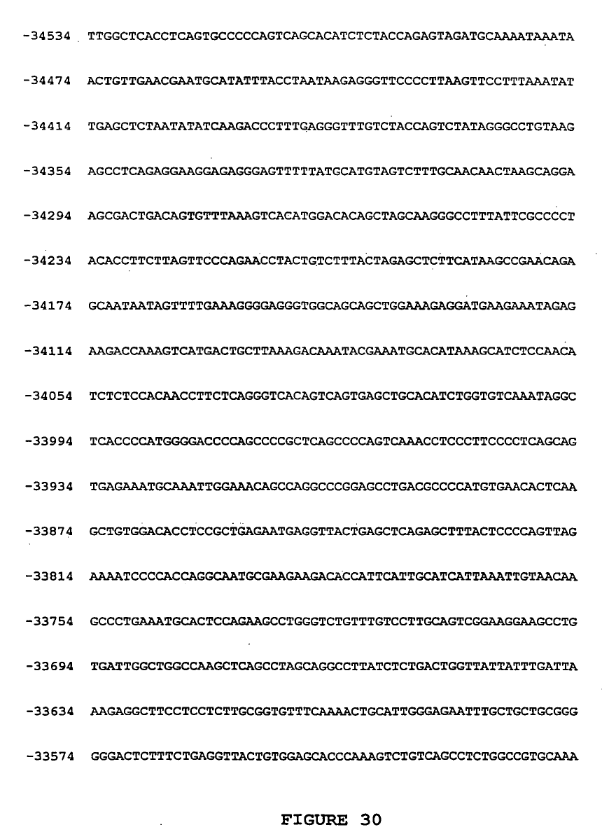

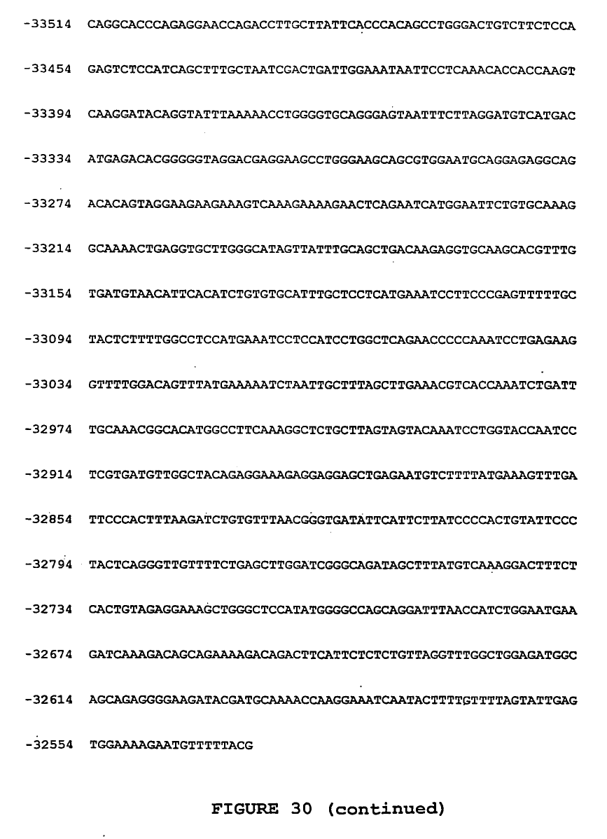

Using bioinformatic techniques, the control region of the human GPAT gene was identified and mapped out. By searching GenBank's EST division using BLASTN with the human GPAT CDS, a human EST containing 205 bp of 5′ UTR for the gene was identified (GenBank Accession No. AV695288). This experiment revealed the presence of two additional upstream exons containing exclusively 5′ UTR, the first intron being over 31 kb in length. The control region between positions-32535 and -34534 from the ATG is shown in FIG. 30.

GPAT catalyzes the acylation of sn-glycerol-3-phosphate to form 1-acyl-sn-glycerol-3-phosphate, thereby providing the committed step for the formation of glycerolipids (Bell R. M. and Coleman R. A., 1980, Annu. Rev. Biochem., 49: 459-487). Antibodies have been generated against the mouse mitochondrial protein (Yet et al., 1993, Biochemistry, 32: 9486-9491).

Using the rat partial cDNA clone as the probe for Northern blot analysis, the present inventors show for the first time that PUFAs decrease GPAT mRNA levels (see Tables 1 and 2).

The present inventors have thus demonstrated a novel drug target, i.e. GPAT, including the use of rat and human genes as well as promoters to screen for novel drugs or chemical entities that modulate expression of the gene or activity of the gene product.

XI. Spot-14 Gene and Polypeptide

One of the differentially expressed bands identified during the study was a rat partial cDNA for the Spot-14 gene. Spot-14 sequences are known in human (Grillasca et al., 1997, FEBS Lett., 401: 38-42, GenBank Accession No. Y08409), rat (Liaw C. W. and Towle H. C., 1984, J. Biol. Chem., 259: 7253-7260, GenBank Accession No. K01934) and mouse (Grillasca et al., 1997, FEBS Lett., 401: 38-42, GenBank Accession No. X95279). Expression of the rat and mouse Spot-14 gene has been extensively studied in liver and adipose tissue. The Spot-14 protein, found in the nuclei of lipogenic tissues using Spot-14-specific antibodies, is induced synergistically by thyroid hormone (T3) and dietary carbohydrate (Kinlaw et al., 1989, J. Biol. Chem., 264: 19779-19783; Kinlaw et al., 1993, Endocrinology, 133: 645-650; Clarke et al., 1990, J. Nutr., 120: 218-224; Jump et al., 1994, J. Lipid Res., 35: 1076-1084 and Liu H. C. and Towle H. C., 1994, Mol. Endocrinol., 8: 1021-1037).

Spot-14 has been implicated in lipogenesis, more specifically being a metabolic integrator that increases lipogenesis in normal and cancerous tissue (Cunningham et al., 1998, Thyroid, 8: 815-825). The THRSP gene is expressed in human liver and adipocytes, particularly in lipomatous nodules. Moreover, its expression is altered in streptozotocin-induced diabetes (Jump et al., 1990, Mol. Endocrinol., 4: 1655-1660) which is consistent with the present inventors' results that suggest mRNA abundance is reduced in STZ-induced diabetic rats. The present inventors' Northern blot data (Table 2) are consistent with data reported previously showing that PUFAs decrease Spot-14 mRNA levels (Foretz et al., 1999, Biochem. J., 341: 371-376).

Grillasca et al. (1997, FEBS Lett., 401: 38-42) cloned the human and mouse genes (also called THRSP) and promoters, and found that it has the same organization as the rat gene and predicts a highly conserved amino acid sequence (more than 80%). Taviaux et al. (1997, Cylogenet. Cell Genet., 76: 219-220) mapped the THRSP gene to 11q13.5-q14.1 by fluorescence in situ hybridization.

Enhanced long-chain fatty acid synthesis may occur in breast cancer. It is necessary for tumor growth indicative of a poor prognosis. The Spot-14 protein activates genes encoding the enzymes of fatty acid synthesis. Amplification of chromosome region 11q13, where the THRSP gene resides, also predicts a poor prognosis in breast tumors. Moncur et al. (1998, Proc. Natl. Acad. Sci., 95: 6989-6994) localized the THRSP gene between markers D11S906 and D11S937, at the telomeric end of the amplified region at 11q13, and found that it was amplified and expressed in breast cancer-derived cell lines. Other findings supported a role for the protein as a determinant of tumor lipid metabolism. Expression of Spot-14 provided a pathophysiologic link between 2 prognostic indicators in breast cancer: enhanced lipogenesis and 11q13 amplification.

The present invention teaches a method for identifying test compounds for a drug target, i.e. Spot-14, which includes the use of rat and human genes as well as promoters to screen for novel drugs or chemical entities that modulate expression of the gene or activity of the gene product.

XII. Catechol-O-Methyltransferase Gene and Polypeptide

One of the differentially expressed bands identified during the study was a rat partial cDNA for the catechol-O-methyltransferase (COMT) gene (Salminen et al., 1990, Gene, 93: 241-247 and Lundstrom et al., 1991, DNA Cell Biol., 10: 181-189).

COMT inactivates a variety of catechol containing molecules in different organisms by methylating one of the phenolic hydroxyl groups in the substrate (Guldberg and Marsden, 1975, Pharmacol, Rev., 27: 135-306). In mammals, neurotransmitters, hormones, and drugs containing the catechol moiety, such as L-DOPA used in the treatment of Parkinson's disease, are inactivated by COMT (Tenhunen J., 1996, DNA Cell Biol. 15: 461-473). Inhibitors of COMT are believed to be beneficial for the treatment of Parkinson's and Alzheimer's diseases (Jorga et al., 2000, Clin. Pharmacol. Ther., 67: 610-620 and Chong B. S. and Mersfelder T. L., 2000, Ann. Pharmacother., 34: 1056-1065).

The human and rat COMT genes and promoters have been described (Tenhunen et al., 1994, Eur. J. Biochem., 223: 1049-1059; Xie et al., 1999, Mol. Pharmacol., 56: 31-38 and Tenhunen J., 1996, DNA Cell Biol., 15: 461-473). COMT has been purified and antibodies against it have been described (Tilgmann C. and Ulmanen I., 1996, J. Chromatogr. B Biomed. Appl., 684: 147-461 and Kastner et al., 1994, Neuroscience, 62: 449-457).

Enzymatic assays for COMT using adrenaline as substrate, have been published and are well known by persons skilled in the art (Vieira-Coelho M. and Soares-da-Silva P., 1999, Brain Res., 821: 69-78 and Borges et al., 1998, J. Enzyme Inhib. 13: 473-483).

Using the rat partial cDNA clone as the probe for Northern blotting, the present inventors show for the first time that PUFAs decrease COMT mRNA levels (see Tables 1 and 2).

The present inventors have thus demonstrated a novel method for identifying test compounds which target COMT expression, which includes the use of rat and human genes as well as promoters to screen for novel drugs or chemical entities that modulate expression of the gene or activity of the gene product.

XIII INSIG1 Gene and Polypeptide

One of the differentially expressed bands identified during the study was a rat partial cDNA for the insulin-induced gene 1 (INSIG1). INSIG1 sequences are known in human (Peng et al., 1997, Genomics, 43: 278-284, GenBank Accession No. U96876) and rat where it was originally named CL-6 (Diamond et al., 1993, J. Biol. Chem., 268: 15185-15192, GenBank Accession No. L13619).

Rat CL-6 is the most highly insulin-induced gene in a liver cell line and expressed in proliferating liver during regeneration and development (Bortoff et al., 1997, Endocrine, 7: 199-207; Chin et al., 1995, Am. J. Physiol., 269: E691-E700 and Haber et al., 1993, Hepatology, 22: 906-914).

The human gene shares 80% identity with the rat gene within the translated region. By fluorescence in situ hybridization the gene was mapped to 7q36 (Peng et al., 1997, Genomics, 43: 278-284). The predicted molecular weights for the human and rat proteins are 30 and 28 kDa, respectively. INSIG1 has five potential membrane spanning domains and an experimental molecular weight of approximately 43 kDa (Diamond et al., 1993, J. Biol. Chem., 268: 15185-15192) determined by using anti-CL-6 antisera in Western blots against the rat protein.

INSIG1 is known to be involved in liver regeneration and is highly induced by insulin. The protein has no clear homology to functional domains of other proteins. The highest expression is in liver and kidney followed by heart and muscle (Diamond et al., 1993, J. Biol. Chem., 268: 15185-15192). Promoter studies have been conducted with human INSIG1 using luciferase assay (Peng et al., 1997, Genomics, 43: 278-284).

Using the rat partial cDNA clone as the probe for Northern blotting the present inventors show for the first time that PUFAs decrease INSIG1 mRNA levels (see Tables 1 and 2).

The present inventors have thus demonstrated a novel method for identifying test compounds which target. INSIG1 expression, which includes the use of rat and human genes as well as promoters to screen for novel drugs or chemical entities that modulate expression of the gene or activity of the gene product.

Subject Polynucleotides and Polypeptides

The subject polynucleotides and polypeptides may be employed as research reagents and materials for discovery of treatments of and diagnostics for disease, particularly human disease, as further discussed herein.

Nucleotide Probes

The nucleic acid molecules of the invention allow those skilled in the art to construct nucleotide probes for use in the detection of nucleotide sequences in biological materials. As described herein, a number of unique restriction sequences for restriction enzymes are incorporated in the nucleic acid molecule identified in the sequence listings of the subject polynucleotides, and these provide access to nucleotide sequences which code for polypeptides unique to the subject polynucleotides of the invention. Nucleotide sequences unique to the subject polynucleotides or isoforms thereof can also be constructed by chemical synthesis and enzymatic ligation reactions carried out by procedures known in the art.

A nucleotide probe may be labeled with a detectable marker such as a radioactive label which provides for an adequate signal and has sufficient half-life such as ³²P, ³H, 14C or the like. Other detectable markers which may be used include antigens that are recognized by a specific labeled antibody, fluorescent compounds, enzymes, antibodies specific for a labeled antigen, and chemiluminescent compounds. An appropriate label may be selected with regard to the rate of hybridization and binding of the probe to the nucleotide to be detected and the amount of nucleotide available for hybridization. The nucleotide probes may be used to detect genes related to or analogous to the subject polynucleotides of the invention.

Accordingly, the present invention also provides a method of detecting the presence of nucleic acid molecules encoding a polypeptide related to or analogous to the subject polynucleotides in a sample comprising contacting the sample under hybridization conditions with one or more of the nucleotide probes of the invention labeled with a detectable marker, and determining the degree of hybridization between the nucleic acid molecule in the sample and the nucleotide probes.

Hybridization conditions which may be used in the method of the invention are known in the art and are described for example in Sambrook et al., supra. The hybridization product may be assayed using techniques known in the art. The nucleotide probe may be labeled with a detectable marker as described herein and the hybridization product may be assayed by detecting the detectable marker or the detectable change produced by the detectable marker.

Primers

The identification of the nucleic acid molecule of the invention also permits the identification and isolation, or synthesis of nucleotide sequences which may be used as primers to amplify a polynucleotide molecule of the invention, for example in polymerase chain reaction (PCR). The length and bases of the primers for use in the PCR are selected so that they will hybridize to different strands of the desired sequence and at relative positions along the sequence such that an extension product synthesized from one primer when it is separated from its template can serve as a template for extension of the other primer into a nucleic acid of defined length.

Primers which may be used in the invention are oligonucleotides i.e. molecules containing two or more deoxyribonucleotides of the nucleic acid molecule of the invention which occur naturally as in a purified restriction endonuclease digest or are produced synthetically using techniques known in the art such as, for example, phosphotriester and phosphodiester methods or automated techniques (see, Connolly B. A., 1987, Nucl. Acid Res., 15: 3131-3139). The primers are capable of acting as a point of initiation of synthesis when placed under conditions which permit the synthesis of a primer extension product which is complementary to the DNA sequence of the invention e.g. in the presence of nucleotide substrates, an agent for polymerization such as DNA polymerase and at suitable temperature and pH. Preferably, the primers are sequences that do not form secondary structures by base pairing with other copies of the primer or sequences that form a hair pin configuration. The primer may be single or double-stranded. When the primer is double-stranded it may be treated to separate its strands before using it to prepare amplification products. The primer preferably contains between about 7 and 25 nucleotides.

The primers may be labeled with detectable markers which allow for detection of the amplified products. Suitable detectable markers are radioactive markers such as ³²P, ³⁵S, ¹²⁵I and ³H, luminescent markers such as chemiluminescent markers, preferably luminol and fluorescent markers, preferably dansyl chloride, fluorescein-5-isothiocyanate and 4-fluor-7-nitrobenz-2-oxa-1,3 diazole and cofactors such as biotin. It will be appreciated that the primers may contain non-complementary sequences provided that a sufficient amount of the primer contains a sequence which is complementary to a nucleic acid molecule of the invention or oligonucleotide sequence thereof, which is to be amplified. Restriction site linkers may also be incorporated into the primers allowing for digestion of the amplified products with the appropriate restriction enzymes facilitating cloning and sequencing of the amplified product.

Assays—Amplifying Sequences

Thus, a method of determining the presence of a nucleic acid molecule having a sequence encoding the subject polynucleotides or a predetermined oligonucleotide fragment thereof in a sample, is provided comprising treating the sample with primers which are capable of amplifying the nucleic acid molecule or the predetermined oligonucleotide fragment thereof in a polymerase chain reaction to form amplified sequences, under conditions which permit the formation of amplified sequences and, assaying for amplified sequences.

The polymerase chain reaction refers to a process for amplifying a target nucleic acid sequence as generally described in Innis M. A. and Gelfand D. H., 1989, PCR Protocols, A Guide to Methods and Applications, Innis M. A., Gelfand D. H., Shinsky J. J. and White T. J. (eds), Academic Press, NY, pp. 3-12, which are incorporated herein by reference. Conditions for amplifying a nucleic acid template are described in Innis M. A. and Gelfand D. H., 1989, PCR Protocols, A Guide to Methods and Applications, Innis M. A., Gelfand D. H., Shinsky J. J. and White T. J. (eds), Academic Press, NY, pp. 3-12, which is also incorporated herein by reference.

The amplified products can be isolated and distinguished based on their respective sizes using techniques known in the art. For example, after amplification, the DNA sample can be separated on an agarose gel and visualized, after staining with ethidium bromide, under ultraviolet (UV) light. DNA may be amplified to a desired level and a further extension reaction may be performed to incorporate nucleotide derivatives having detectable markers such as radioactive labeled or biotin labeled nucleoside triphosphates. The primers may also be labeled with detectable markers. The detectable markers may be analyzed by restriction and electrophoretic separation or other techniques known in the art.

The conditions which may be employed in the methods of the invention using PCR are those which permit hybridization and amplification reactions to proceed in the presence of DNA in a sample and appropriate complementary hybridization primers. Conditions suitable for the polymerase chain reaction are generally known in the art. For example, see Innis M. A. and Gelfand D. H., 1989, PCR Protocols, A Guide to Methods and Applications, Innis M. A., Gelfand D. H., Shinsky J. J. and White T. J. (eds), Academic Press, NY, pp. 3-12, which is incorporated herein by reference. Preferably, the PCR utilizes polymerase obtained from thermophilic bacterium Thermus aquaticus (Taq polymerase, GeneAmp Kit, Perkin Elmer Cetus) or other thermostable polymerase may be used to amplify DNA template strands.

It will be appreciated that other techniques such as the Ligase Chain Reaction (LCR) and Nucleic-Acid Sequence Based Amplification (NASBA) may be used to amplify a nucleic acid molecule of the invention. In LCR, two primers which hybridize adjacent to each other on the target strand are ligated in the presence of the target strand to produce a complementary strand (Backman, 1991 and European Published Application No. 0320308, published Jun. 14, 1989). NASBA is a continuous amplification method using two primers, one incorporating a promoter sequence recognized by an RNA polymerase and the second derived from the complementary sequence of the target sequence to the first primer (U.S. Pat. No. 5,130,238 to Malek).

Vectors

The present invention also teaches vectors which comprise a polynucleotide or polynucleotides of the present invention, host cells which are genetically engineered with vectors of the invention and the production of polynucleotides of the invention by recombinant techniques.

In accordance with this aspect of the invention the vector may be, for example, a plasmid vector, a single or double-stranded phage vector, or a single or double-stranded RNA or DNA viral vector. In certain embodiments in this regard, the vectors provide for specific expression. Such specific expression may be inducible expression or expression only in certain types of cells or both inducible and cell-specific. Particular among inducible vectors are vectors that can be induced for expression by environmental factors that are easy to manipulate, such as temperature and nutrient additives. A variety of vectors suitable to this aspect of the invention, including constitutive and inducible expression vectors for use in prokaryotic and eukaryotic hosts, are well known and employed routinely by those of skill in the art. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. All of these may be used for expression in accordance with this aspect of the present invention.

The following vectors, which are commercially available, are provided by way of example. Among vectors for use in bacteria are pQE-9, pQE-16, pQE-30, pQE-40, pQE-50 and pQE-60 (Qiagen); pCRII, pCRII-TOPO, pTrcHis and pBAD-TOPO (Invitrogen); pGEM-3Z, pGEMEX-1, pET-5 (Promega); pBS phagemid vectors, Phagescript vectors, Bluescript vectors, pCAL, pET-3 and pSPUTK (Stratagene); pTrc99A, pKK223-3, pKK232-8and pRIT2T (Pharmacia); pMAL (New England Biolabs); and pBR322 (ATCC 37017). Among eukaryotic vectors are pGAPZ, pYES2, pYES2/CT and pcDNA3.1(Invitrogen); pCAT3 and pGL3 (Promega); pCMV-Script, pXT1, pDual, pCMVLacI, pESC, HybriZAP2.1, ImmunoZAP and pRS (Stratagene); and pSVK3, pSVL and pMSG (Pharmacia). These vectors are listed solely by way of illustration of the many commercially available and well known vectors that are available to those of skill in the art for use in accordance with this aspect of the present invention. It will be appreciated that any other plasmid or vector suitable for, for example, introduction, maintenance, propagation or expression of a polynucleotide or polypeptide of the invention in a host may be used in this aspect of the invention. Generally, any vector suitable to maintain, propagate or express polynucleotides to express a polypeptide or polynucleotide in a host may be used for expression in this regard.

The DNA sequence in the expression vector is operatively linked to appropriate expression control sequence(s), including, for instance, a promoter to direct mRNA transcription. Promoter regions can be selected from any desired gene using vectors that contain a reporter transcription unit lacking a promoter region, such as a chloramphenicol acetyl transferase (CAT) transcription unit, downstream of restriction site or sites for introducing a candidate promoter fragment; i.e., a fragment that may contain a promoter. As is well known, introduction into the vector of a promoter-containing fragment at the restriction site upstream of the CAT gene engenders production of CAT activity, which can be detected by standard CAT assays. Vectors suitable to this end are well known and readily available, such as pKK232-8 and pCAT3. Promoters for expression of polynucleotides of the present invention include not only well known and readily available promoters, but also promoters that readily may be obtained by the foregoing technique, using a reporter gene. Among known prokaryotic promoters suitable for expression of polynucleotides and polypeptides in accordance with the present invention are the E. coli lacI and lacZ promoters, the T3 and T7 promoters, the gpt promoter, the lambda PR and PL promoters, and the trp promoter. Among known eukaryotic promoters suitable in this regard are the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

Vectors for propagation and expression generally will include selectable markers and amplification regions, such as, for example, those set forth in Sambrook et al., supra.

Host Cells

As hereinbefore mentioned, the present invention also teaches host cells which are genetically engineered with vectors of the invention.

Polynucleotide constructs in host cells can be used in a conventional manner to produce the gene product encoded by the recombinant sequence. The subject polynucleotides or polypeptides products or isoforms or parts thereof, may be obtained by expression in a suitable host cell using techniques known in the art. Suitable host cells include prokaryotic or eukaryotic organisms or cell lines, for example bacterial, mammalian, yeast, or other fungi, viral, plant or insect cells. Methods for transforming or transfecting cells to express foreign DNA are well known in the art (See for example, Itakura et al., U.S. Pat. No. 4,704,362; Hinnen et al., 1978; Murray et al., U.S. Pat. No. 4,801,542; McKnight et al., U.S. Pat. No. 4,935,349; Hagen et al., U.S. Pat. No. 4,784,950; Axel et al., U.S. Pat. No. 4,399,216; Goeddal et al., U.S. Pat. No. 4,766,075 and Sambrook et al., 1989, Molecular Cloning, 2nd Edition, Cold Spring Harbor Laboratory Press, Cold Spring Harbour, N.Y. all of which are incorporated herein by reference). Representative examples of appropriate hosts include bacterial cells, such as Streptococci, Staphylococci, E. coli, Streptomyces and Bacillus subtilis; fungal cells, such as yeast cells and Aspergillus cells; insect cells such as Drosophila S2 and Spodoptera Sf9 cells; animal cells such as CHO, COS-1, ZR-75-1, Chang, HeLa, C127, 3T3, HepG2, BHK, 293 and Bowes melanoma cells; and plant cells.

Host cells can be genetically engineered to incorporate polynucleotides and express polynucleotides of the present invention. Introduction of polynucleotides into the host cell can be effected by calcium phosphate transfection, DEAE-dextran mediated transfection, transvection, microinjection, cationic lipid-mediated transfection, electroporation, transduction, scrape loading, ballistic introduction, infection or other methods. Such methods are described in many standard laboratory manuals, such as Davis et al., 1986, Basic Methods in Molecular Biology, Elsevier, N.Y. and Sambrook et al., 1989, Molecular Cloning, 2nd Edition, Cold Spring Harbor Laboratory Press, Cold Spring Harbour, N.Y.

Production of the Subject Polypeptides

As hereinbefore mentioned, the present invention also teaches the production of polynucleotides of the invention by recombinant techniques.

The subject polynucleotides encode polypeptides which are the mature protein plus additional amino-or carboxyl-terminal amino acids, or amino acids interior to the mature polypeptide (when the mature form has more than one polypeptide chain, for instance). Such sequences may play a role in processing of a protein from precursor to a mature form, may allow protein transport, may lengthen or shorten protein half-life or may facilitate manipulation of a protein for assay or production, among other things. Generally, as is the case in vivo, the additional amino acids may be processed away from the mature protein by cellular enzymes.

A precursor protein, having the mature form of the polypeptide fused to one or more prosequences may be an inactive form of the polypeptide. When prosequences are removed such inactive precursors generally are activated. Some or all of the prosequences may be removed before activation. Generally, such precursors are called proproteins.

Thus, a polynucleotide of the present invention may encode a mature protein, a mature protein plus a leader sequence (which may be referred to as a preprotein), a precursor of a mature protein having one or more prosequences which are not the leader sequences of a preprotein, or a preproprotein, which is a precursor to a proprotein, having a leader sequence and one or more prosequences, which generally are removed during processing steps that produce active and mature forms of the polypeptide.

The polypeptides of the invention may be prepared by culturing the host/vector systems described above, in order to express the recombinant polypeptides. Recombinantly produced subject protein or parts thereof, may be further purified using techniques known in the art such as commercially available protein concentration systems, by salting out the protein followed by dialysis, by affinity chromatography, or using anion or cation exchange resins.

Mature proteins can be expressed in mammalian cells, yeast, bacteria, or other cells under the control of appropriate promoters. Cell-free translation systems can also be employed to produce such proteins using DNA derived from the DNA constructs of the present invention. Appropriate cloning and expression vectors for use with prokaryotic and eukaryotic hosts are described by Sambrook et al., supra.

Polynucleotides of the invention, encoding the heterologous structural sequence of a polynucleotide or polypeptide of the invention generally will be inserted into a vector using standard techniques so that it is operably linked to the promoter for expression. The polynucleotide will be positioned so that the transcription start site is located appropriately 5′ to a ribosome binding site. The ribosome binding site will be 5′ to the AUG that initiates translation of the polynucleotide or polypeptide to be expressed. Generally, there will be no other open reading frames that begin with an initiation codon, usually AUG, and lie between the ribosome binding site and the initiation codon. Also, generally, there will be a translation stop codon at the end of the expressed polynucleotide and there will be a polyadenylation signal in constructs for use in eukaryotic hosts. Transcription termination signal appropriately disposed at the 3′ end of the transcribed region may also be included in the polynucleotide construct.

For secretion of the translated protein into the lumen of the endoplasmic reticulum, into the periplasmic space or into the extracellular environment, appropriate secretion signals may be incorporated into the expressed polynucleotide or polypeptide. These signals may be endogenous to the polynucleotide or they may be heterologous signals. Microbial cells employed in expression of proteins can be disrupted by any convenient method, including freeze-thaw cycling, sonication, mechanical disruption, or use of cell lysing agents, or other such methods know to those skilled in the art. A subject polynucleotide or polypeptide can be recovered and purified from recombinant cell cultures by known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polynucleotide is denatured during isolation and or purification.

A nucleic acid molecule of the invention may be cloned into a glutathione S-transferase (GST) gene fusion system for example the pGEX-1T, pGEX-2T and pGEX-3X of Pharmacia. The fused gene may contain a strong lac promoter, inducible to a high level of expression by IPTG, as a regulatory element. Thrombin or factor Xa cleavage sites may be present which allow proteolytic cleavage of the desired polypeptide from the fusion product. The glutathione S-transferase-subject polypeptide fusion protein may be easily purified using a glutathione sepharose 4B column, for example from Pharmacia. The 26 kDa glutathione S-transferase polypeptide can be cleaved by thrombin (pGEX-1T or pGEX-2T) or factor Xa (pGEX-3X) and resolved from the polypeptide using the same affinity column. Additional chromatographic steps can be included if necessary, for example Sephadex or DEAE cellulose. The two enzymes may be monitored by protein and enzymatic assays and purity may be confirmed using SDS-PAGE.

The subject protein or parts thereof may also be prepared by chemical synthesis using techniques well known in the chemistry of proteins such as solid phase synthesis (Merrifield, 1964, J. Am. Chem. Assoc., 85: 2149-2154) or synthesis in homogenous solution (Houbenweyl et al., 1987, Methods of Organic Chemistry, Wansch E. (ed), Vol. 15 I and II, Thieme, Germany).

Within the context of the present invention, the subject polypeptide includes various structural forms of the primary protein which retain biological activity. For example, the subject polypeptide may be in the form of acidic or basic salts or in neutral form. In addition, individual amino acid residues may be modified by oxidation or reduction. Furthermore, various substitutions, deletions or additions may be made to the amino acid or nucleic acid sequences, the net effect being that biological activity of the subject polypeptide is retained. Due to code degeneracy, for example, there may be considerable variation in nucleotide sequences encoding the same amino acid.

The polypeptide may be expressed in a modified form, such as a fusion protein, and may include not only secretion signals but also additional heterologous functional regions. Thus, for instance, a region of additional amino acids, particularly charged amino acids, may be added to the carboxyl- or amino-terminus of the polypeptide to improve stability and persistence in the host cell during purification or during subsequent handling and storage. Also, fusion proteins may be added to the polynucleotide or polypeptide to facilitate purification. Such regions may be removed prior to final preparation of the polynucleotide or polypeptide. The addition of peptide moieties to polynucleotides or polypeptides to engender secretion or excretion, to improve stability or to facilitate purification, among others, are familiar and routine techniques in the art. In drug discovery, for example, proteins have been fused with antibody Fc portions for the purpose of high-throughput screening assays to identify antagonists (see Bennett et al., 1995, J. Mol. Recognit., 8: 52-58, and Johanson et al., 1995, J. Biol. Chem., 270: 9459-9471).

Antibodies

With respect to protein-based testing, antibodies can be generated to the fat regulated gene product using standard immunological techniques, fusion proteins or synthetic peptides as described herein. Monoclonal antibodies can also be produced using now conventional techniques such as those described in Waldmann T. A., 1991, Science, 252: 1657-1662 and Harlow E. and Lane D. (eds.), 1988, Antibodies: A Laboratory Manual, Cold Harbour Press, Cold Harbour, N.Y. It will also be appreciated that antibody fragments, i.e. Fab′ fragments, can be similarly employed. Immunoassays, for example ELISAs, in which the test sample is contacted with antibody and binding to the gene product detected, can provide a quick and efficient method of determining the presence and quantity of the fatty acid regulated gene product. For example, the antibodies can be used to test the effect of pharmaceuticals in subjects enrolled in clinical trials.

Thus, the present invention also provides polyclonal and/or monoclonal antibodies and fragments thereof, and immunologic binding equivalents thereof, which are capable of specifically binding to the subject polypeptides and fragments thereof or to polynucleotide sequences from the subject polynucleotide region, particularly from the subject polypeptide locus or a portion thereof. The term “antibody” is used both to refer to a homogeneous molecular entity, or a mixture such as a serum product made up of a plurality of different molecular entities. Polypeptides may be prepared synthetically in a peptide synthesizer and coupled to a carrier molecule (e.g., keyhole limpet hemocyanin) and injected over several months into rabbits. Rabbit sera is tested for immunoreactivity to the subject polypeptide or fragment. Monoclonal antibodies may be made by injecting mice with the protein polypeptides, fusion proteins or fragments thereof. Monoclonal antibodies are screened by ELISA and tested for specific immunoreactivity with subject polypeptide or fragments thereof (Harlow E. and Lane D. (eds.), 1988, Antibodies: A Laboratory Manual, Cold Harbour Press, Cold Harbour, N.Y.). These antibodies are useful in assays as well as pharmaceuticals.

Once a sufficient quantity of desired polypeptide has been obtained, it may be used for various purposes. A typical use is the production of antibodies specific for binding. These antibodies may be either polyclonal or monoclonal, and may be produced by in vitro or in vivo techniques well known in the art. For production of polyclonal antibodies, an appropriate target immune system, typically mouse or rabbit, is selected. Substantially purified antigen is presented to the immune system in a fashion determined by methods appropriate for the animal and by other parameters well known to immunologists. Typical routes for injection are in footpads, intramuscularly, intraperitoneally, or intradermally. Of course, other species may be substituted for mouse or rabbit. Polyclonal antibodies are then purified using techniques known in the art, adjusted for the desired specificity.

An immunological response is usually assayed with an immunoassay. Normally, such immunoassays involve some purification of a source of antigen, for example, that produced by the same cells and in the same fashion as the antigen. A variety of immunoassay methods are well known in the art, such as in Harlow E. and Lane D. (eds.), 1988, Antibodies: A Laboratory Manual, Cold Harbour Press, Cold Harbour, N.Y., or Goding J. W., 1996, Monoclonal Antibodies: Principles and Practice: Production and Application of Monoclonal Antibodies in Cell Biology, Biochemistry and Immunology, 3^{rd} edition, Academic Press, NY.

Monoclonal antibodies with affinities of 10⁸ M⁻¹ or preferably 10⁹ to 10¹⁰ M⁻¹ or stronger will typically be made by standard procedures as described in Harlow E. and Lane D. (eds.), 1988, Antibodies: A Laboratory Manual, Cold Harbour Press, Cold Harbour, N.Y. or Goding J. W., 1996, Monoclonal Antibodies: Principles and Practice: Production and Application of Monoclonal Antibodies in Cell Biology, Biochemistry and Immunology, 3^{rd} edition, Academic Press, NY. Briefly, appropriate animals will be selected and the desired immunization protocol followed. After the appropriate period of time, the spleens of such animals are excised and individual spleen cells fused, typically, to immortalized myeloma cells under appropriate selection conditions. Thereafter, the cells are clonally separated and the supernatants of each clone tested for their production of an appropriate antibody specific for the desired region of the antigen.

Other suitable techniques involve in vitro exposure of lymphocytes to the antigenic polypeptides, or alternatively, to selection of libraries of antibodies in phage or similar vectors (Huse et al., 1989, Science, 246: 1275-1281). The polypeptides and antibodies of the present invention may be used with or without modification. Frequently, polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149 and 4,366,241. Also, recombinant immunoglobulins may be produced (see U.S. Pat. No. 4,816,567).

Generation of Polyclonal Antibody Against the Subject Polynucleotide

Segments of the subject polynucleotide coding sequence are expressed as fusion protein in E. coli. The overexpressed protein is purified by gel elution and used to immunize rabbits and mice using a procedure similar to the one described by Harlow E. and Lane D. (eds.), 1988, Antibodies: A Laboratory Manual, Cold Harbour Press, Cold Harbour, N.Y. This procedure has been shown to generate antibodies against various other proteins (for example, see Kraemer et al., 1993, J. Lipid Res., 34: 663-671).

Briefly, a stretch of coding sequence selected from the subject polynucleotide is cloned as a fusion protein in plasmid pET5A (Novagen, Wis.) or pMAL system (New England Biolabs). After induction with IPTG, the overexpression of a fusion protein with the expected molecular weight is verified by SDS-PAGE. Fusion protein is purified from the gel by electroelution. The identification of the protein as the subject polypeptide fusion product can be verified by protein sequencing at the N-terminus. Next, the purified protein is used as immunogen in rabbits. Rabbits are immunized with 100 μg of the protein in complete Freund's adjuvant and boosted twice in 3 week intervals, first with 100 μg of immunogen in incomplete Freund's adjuvant followed by 100 μg of immunogen in PBS. Antibody containing serum is collected two weeks thereafter.

This procedure is repeated to generate antibodies against the mutant forms of the subject polypeptide. These antibodies, in conjunction with antibodies to wild type subject polypeptide, are used to detect the presence and the relative level of the mutant forms in various tissues and biological fluids.

Generation of Monoclonal Antibodies Specific for the Subject Polypeptide

Monoclonal antibodies are generated according to the following protocol. Mice are immunized with immunogen comprising intact subject polypeptide or its peptides (wild type or mutant) conjugated to keyhole limpet hemocyanin using glutaraldehyde or EDC as is well known.

The immunogen is mixed with an adjuvant. Each mouse receives four injections of 10 to 100 μg of immunogen and after the fourth injection blood samples are taken from the mice to determine if the serum contains antibody to the immunogen. Serum titer is determined by ELISA or RIA. Mice with sera indicating the presence of antibody to the immunogen are selected for hybridoma production.

Spleens are removed from immune mice and a single cell suspension is prepared as described by Harlow E. and Lane D. (eds.), 1988, Antibodies: A Laboratory Manual, Cold Harbour Press, Cold Harbour, N.Y. Cell fusions are performed essentially as described by Kohler G. and Milstein C., 1975, Nature, 256: 495-497. Briefly, P3.65.3 myeloma cells (American Type Culture Collection, Rockville, Md.) are fused with immune spleen cells using polyethylene glycol as described by Harlow E. and Lane D. (eds.), 1988, Antibodies: A Laboratory Manual, Cold Harbour Press, Cold Harbour, N.Y. Cells are plated at a density of 2×10⁵ cells/well in 96 well tissue culture plates. Individual wells are examined for growth and the supernatants of wells with growth are tested for the presence of subject polypeptide specific antibodies by ELISA or RIA using wild type or mutant target protein. Cells in positive wells are expanded and subcloned to establish and confirm monoclonality.

Clones with the desired specificities are expanded and grown as ascites in mice or in a hollow fiber system to produce sufficient quantities of antibody for characterization and assay development.

Sandwich Assay for the Subject Polypeptide

Monoclonal antibody is attached to a solid surface such as a plate, tube, bead, or particle. Preferably, the antibody is attached to the well surface of a 96-well ELISA plate. A 100 μl sample (e.g., serum, urine, tissue cytosol) containing the subject polypeptide/protein (wild-type or mutant) is added to the solid phase antibody. The sample is incubated for 2 hrs at room temperature. Next the sample fluid is decanted, and the solid phase is washed with buffer to remove unbound material. One hundred μl of a second monoclonal antibody (to a different determinant on the subject polypeptide/protein) is added to the solid phase. This antibody is labeled with a detector molecule or atom (e.g., ¹²⁵I, enzyme, fluorophore, or a chromophore) and the solid phase with the second antibody is incubated for two hrs at room temperature. The second antibody is decanted and the solid phase is washed with buffer to remove unbound material.

The amount of bound label, which is proportional to the amount of subject polypeptide/protein present in the sample, is quantitated. Separate assays are performed using monoclonal antibodies which are specific for the wild-type subject polypeptide as well as monoclonal antibodies specific for each of the mutations identified in subject polypeptide.

Detecting Presence of or Predisposition for Disorders Affected by Lipid Metabolism and Monitoring Treatment of Same

As previously discussed, lipid metabolism is frequently disregulated in disease. It is likely that genetic polymorphisms in fat regulated genes will contribute to disease susceptibility.

The subject polynucleotides taught herein are useful to detect genetic polymorphisms of the subject polynucleotides, or to detecting changes in the level of expression of the subject polynucleotides, as a diagnostic tool. Detection of an aberrant form of the subject polynucleotide, or a decrease or increase in the level of expression of the subject polynucleotide in a eukaryote, particularly a mammal, and especially a human, will provide a method for diagnosis of a disease. Eukaryotes (herein also “individual(s)”), particularly mammals, and especially humans, exhibiting genetic polymorphisms of the subject polynucleotides, or changes in expression of the subject polynucleotides may be detected by a variety of techniques.

Since fat regulated genes are widely expressed, test samples of the subject can be obtained from a variety of tissues including blood. A fat regulated gene test can also be included in panels of prenatal tests since fat regulated genes, DNA, RNA or protein can also be assessed in amniotic fluid. Quantitative testing for fat regulated gene transcript and gene product is thus also contemplated within the scope of the present invention.

Nucleic acid and protein-based methods for screening genetic polymorphisms in fat regulated genes are all within the scope of the present teachings. For example, knowing the sequence of the fat regulated gene, DNA or RNA probes can be constructed and used to detect mutations in fat regulated genes through hybridization with genomic DNA in a tissue such as blood using conventional techniques. RNA or cDNA probes can be similarly probed to screen for mutations in fat regulated genes or for quantitative changes in expression. A mixture of different probes, i.e. “probe cocktail”, can also be employed to test for more than one mutation.

With respect to nucleic acid-based testing, genomic DNA may be used directly for detection of a specific sequence or may be amplified enzymatically in vitro by using PCR prior to analysis (Saiki et al., 1985, Science, 230: 1350-1353 and Saiki et al., 1986, Nature, 324: 163-166). Reviews of this subject have been presented by Caskey C. T., 1989, Science, 236: 1223-1228 and by Landegren et al., 1989, Science, 242: 229-237. The detection of specific DNA sequence may be achieved by methods such as hybridization using specific oligonucleotides (Wallace et al., 1986, Cold Spring Harbour Symp. Quant. Biol., 51: 257-261), direct DNA sequencing (Church et al., 1988, Proc. Natl. Acad. Sci., 81: 1991-1995, the use of restriction enzymes (Flavell et al., 1978, Cell, 15: 25-41; Geever et al., 1981, Proc. Natl. Acad. Sci., 78: 5081-5085), discrimination on the basis of electrophoretic mobility in gels with denaturing reagent (Myers et al., 1986, Cold Spring Harbour Sym. Quant. Biol., 51: 275-284), RNase protection (Myers et al., 1985, Science, 230: 1242-1246), chemical cleavage (Cotton et al., 1985, Proc. Natl. Acad. Sci., 85: 4397-4401), and the ligase-mediated detection procedure (Landegren et al., 1988, Science, 241: 1077-1080). Using PCR, characterization of the level of or condition of the subject polynucleotides present in the individual may be made by comparative analysis.

With respect to protein-based testing, antibodies can be generated to the fat regulated gene product using standard immunological techniques, fusion proteins or synthetic peptides as described herein.

With the characterization of the fat regulated gene product and its function, functional assays can also be used for fatty acid regulated gene diagnosis and screening and to monitor treatment. For example, enzymatic testing to determine levels of gene function, rather than direct screening of the fat regulated gene or product, can be employed. Testing of this nature has been utilized in other diseases and conditions, such as in Tay-Sachs.

The invention thus provides a process for detecting disease by using methods known in the art and methods described herein to detect changes in expression of or mutations to the subject polynucleotides. For example, decreased expression of a subject polynucleotide can be measured using any one of the methods well known in the art for the quantification of polynucleotides, such as, for example, PCR, RT-PCR, DNase protection, Northern blotting and other hybridization methods. Thus, the present invention provides a method for detecting disorders affected by lipid metabolism, and a method for detecting a genetic pre-disposition for such diseases including eczema, cardiovascular disorders (including but not limited to hypertriglyceridemia, dyslipidemia, atherosclerosis, coronary artery disease, cerebrovascular disease and peripheral vascular disease), inflammation (including but not limted to sinusitis, asthma, pancreatitis, osteoarthritis, rheumatoid arthritis and acne), body weight disorders (including but not limted to obesity, cachexia and anorexia), psychiatric disorders, cancer, cystic fibrosis, pre-menstrual syndrome, diabetes and diabetic complications.

Drug Screening Assays

The present teachings provide methods for screening compounds to identify those which enhance (agonist) or block (antagonist) the action of subject polypeptides or polynucleotides, such as its interaction with fatty acid binding molecules. The identification of the subject polynucleotides in inherited fatty acid disorders, combined with advances in the field of transgenic methods, provides the information necessary to further study human diseases. This is extraordinarily useful in modeling familial forms of fatty acid disorders and other diseases of fatty acid metabolism including eczema, cardiovascular disorders (including but not limited to hypertriglyceridemia, dyslipidemia, atherosclerosis, coronary artery disease, cerebrovascular disease and peripheral vascular disease), inflammation (including but not limted to sinusitis, asthma, pancreatitis, osteoarthritis, rheumatoid arthritis and acne), body weight disorders (including but not limted to obesity, cachexia and anorexia), psychiatric disorders, cancer, cystic fibrosis, pre-menstrual syndrome, diabetes and diabetic complications. Drug screening assays are made effective by use of the control regions of the genes described in the present invention or part of it, in a yeast based DNA-protein interaction assay (yeast one-hybrid). The use of the genes described here, or parts thereof, or the transcribed RNA in a yeast protein-protein interaction (2-hybrid) or protein-RNA interaction assays for drug screening also provide effective drug screening methods. Such interacting molecules can also be reconstructed in vitro for drug screening purposes.

For example, to screen for agonists or antagonists, a synthetic reaction mix, a cellular compartment, such as a membrane, cell envelope or cell wall, or a preparation of any thereof, may be prepared from a cell that expresses a molecule that binds a subject polynucleotide. The preparation is incubated with labeled polynucleotide in the absence or the presence of a candidate molecule which may be an agonist or antagonist. The ability of the candidate molecule to bind the binding molecule is reflected in decreased binding of the labeled ligand.

Fatty acid-like effects of potential agonists and antagonists may by measured, for instance, by determining activity of a reporter system following interaction of the candidate molecule with a cell or appropriate cell preparation, and comparing the effect to a baseline (control) measurement. Reporter systems that may be useful in this regard include, but are not limited to, colorimetric labeled substrate converted into product, a reporter gene that is responsive to changes in fatty acid enzyme activity, and binding assays known in the art.

Another example of an assay for antagonists is a competitive assay that combines a subject polypeptide and a potential antagonist with membrane-bound subject polypeptide-binding molecules, recombinant subject polypeptide binding molecules, natural substrates or ligands, or substrate or ligand mimetics, under appropriate conditions for a competitive inhibition assay. A subject polypeptide can be labeled, such as by radioactivity or a colorimetric compound, such that the number of subject polypeptide molecules bound to a binding molecule or converted to product can be determined accurately to assess the effectiveness of the potential antagonist.

Potential antagonists include small organic molecules, peptides, polypeptides and antibodies that bind to a polynucleotide or polypeptide of the invention and thereby inhibit or extinguish its activity. Potential antagonists also may be small organic molecules, peptides, polypeptides, such as closely related proteins or antibodies that bind the same sites on a binding molecule, without inducing subject polypeptide-induced activities, thereby preventing the action of the subject polypeptide by excluding the subject polypeptide from binding. Potential antagonists include antisense molecules (Okano et al., 1988, EMBO J., 7: 3407-3412). Potential antagonists include compounds related to and derivatives of the subject polypeptides.

Potential antagonists include small organic molecules, peptides, polypeptides and antibodies that bind to a polynucleotide or polypeptide of the invention and thereby inhibit or extinguish its activity. Potential agonists may be selected from the group consisting of small organic molecules, peptides, polypeptides, antisense molecules, oligonucleotides, polynucleotides, fatty acids, and chemical and functional derivatives thereof.

Developing modulators of the biological activities of specific fat regulated proteins requires differentiating fat regulated isozymes present in a particular assay preparation. The classical enzymological approach of isolating fat regulated proteins from natural tissue sources and studying each new isozyme may be used. Another approach has been to identify assay conditions which might favor the contribution of one isozyme and minimize the contribution of others in a preparation. Still another approach is the separation of fat regulated proteins by immunological means. Each of the foregoing approaches for differentiating fat regulated isozymes is time consuming. As a result many attempts to develop selective fat regulated protein modulators have been performed with preparations containing more than one isozyme. Moreover, fat regulated protein preparations from natural tissue sources are susceptible to limited proteolysis and may contain mixtures of active proteolytic products that have different kinetic, regulatory and physiological properties than the full length fat regulated proteins.

Recombinant subject polypeptide products of the invention greatly facilitate the development of new and specific modulators. The need for purification of an isozyme can be avoided by expressing it recombinantly in a host cell that lacks endogenous fat regulated protein activity. Once a compound that modulates the activity of the fat regulated protein is discovered, its selectivity can be evaluated by comparing its activity on the particular subject enzyme to its activity on other fat regulated isozymes. Thus, the combination of the recombinant subject polypeptide products of the invention with other recombinant fat regulated protein products in a series of independent assays provides a system for developing selective modulators of particular fat regulated proteins. Selective modulators may include, for example, antibodies and other proteins or peptides which specifically bind to the subject polypeptide or polynucleotide, oligonucleotides which specifically bind to the subject polypeptide (see Patent Cooperation Treaty International Publication No. WO 93/05182 which describes methods for selecting oligonucleotides which selectively bind to target biomolecules) or the subject polynucleotide (e.g., antisense oligonucleotides) and other non-peptide natural or synthetic compounds which specifically bind to the subject polynucleotide or polypeptide. Mutant forms of the subject polynucleotide which alter the enzymatic activity of the subject polypeptide or its localization in a cell are also contemplated. Crystallization of recombinant subject polypeptides alone and bound to a modulator, analysis of atomic structure by X-ray crystallography, and computer modeling of those structures are methods useful for designing and optimizing non-peptide selective modulators. See, for example, Erickson et al., 1992, Ann. Rep. Med. Chem., 27: 271-289 for a general review of structure-based drug design.

Targets for the development of selective modulators include, for example: (1) the regions of the subject fat regulated proteins which contact other proteins and/or localize the proteins within a cell, (2) the regions of the proteins which bind substrate, and (3) the phosphorylation site(s) of the subject polypeptides.

Thus, the present invention provides methods for screening and selecting compounds which promote disorders affected by lipids. As well, the present invention provides methods for screening and selecting compounds which treat or inhibit progression of diseases associated with lipid metabolism, such eczema, cardiovascular disorders (including but not limited to hypertriglyceridemia, dyslipidemia, atherosclerosis, coronary artery disease, cerebrovascular disease and peripheral vascular disease), inflammation (including but not limted to sinusitis, asthma, pancreatitis, osteoarthritis, rheumatoid arthritis and acne), body weight disorders (including but not limted to obesity, cachexia and anorexia), psychiatric disorders, cancer, cystic fibrosis, pre-menstrual syndrome, diabetes and diabetic complications, and other diseases not necessarily related to lipid metabolism.

Protein Interaction Assays for DNA Control Regions, CDS and RNA of Fat Regulated Genes.

Protein interaction is implicated in virtually every biological process in the cell, for example, metabolism, transport, signaling and disease. Development of the yeast 2-hybrid and 1-hybrid systems have made it possible to study and identify protein-protein interaction, protein-DNA interaction or protein-RNA interaction in vivo (Fields S. and Song O., 1989, Nature, 340: 245-246; Ulmasov et al., 1997, Science, 276: 1865-1868; Furuyama K. and Sassa S., 2000, J. Clin. Invest., 105: 757-764 and Gyuris et al., 1993, Cell, 75: 791-803). Because these interactions are key to cellular functions, identification of interacting partners is the first step towards elucidation of function and involvement in pathogenesis. New chemical entities that modulate (inhibit or activate) such interactions may have strong pharmaceutical and therapeutic benefit in human, animal as well as plant diseases. It is now known that in sideroblastic anemic patients, the interaction between succinyl-CoA synthetase and the heme biosynthetic enzyme δ-aminolevulinate synthase-E (ALAS-E) is disrupted (Furuyama K. and Sassa S., 2000, J. Clin. Invest., 105: 757-764). Inhibition of gene expression in human cells through small molecule-RNA interaction have been recently described (Hwang et al., 1999, Proc. Natl. Acad. Sci., 96: 12997-13002). The use of protein-RNA inhibition technology is a potential approach for development of anti-HIV therapeutics (Hamy et al., 1997, Proc. Natl. Acad. Sci., 94: 3548-3553 and Mei et al., 1998, Biochemistry, 37: 14204-14212).

Drug Design

Antagonists and agonists and other compounds of the present invention may be employed alone or in conjunction with other compounds, such as therapeutic compounds. The pharmaceutical compositions may be administered in any effective, convenient manner including, for instance, administration by direct microinjection into the affected area, or by intravenous or other routes. These compositions of the present invention may be employed in combination with a non-sterile or sterile carrier or carriers for use with cells, tissues or organisms, such as a pharmaceutical carrier suitable for administration to a subject. Such compositions comprise, for instance, a medium additive or a therapeutically effective amount of antagonists or agonists of the invention and a pharmaceutically acceptable carrier or excipient. Such carriers may include, but are not limited to, saline, buffered saline, dextrose, water, glycerol, ethanol and combinations thereof. The formulation is prepared to suit the mode of administration.

Modulation of fat regulated gene function can be accomplished by the use of therapeutic agents or drugs which can be designed to interact with different aspects of fat regulated metabolic gene protein structure or function. For example, a drug or antibody can bind to a structural fold of the protein to correct a defective structure. Alternatively, a drug might bind to a specific functional residue and increase its affinity for a substrate or cofactor. Efficacy of a drug or agent can be identified by a screening program in which modulation is monitored in vitro in cell systems in which a defective fat regulated gene protein is expressed.

Alternatively, drugs can be designed to modulate the activity of proteins of fat regulated genes from knowledge of the structure and function correlations for such proteins and from knowledge of the specific defect in various mutant proteins (Copsey et al., 1988, Genetically Engineered Human Therapeutic Drugs, Stockton Press, NY).

Gene Therapy

A variety of gene therapy approaches may be used in accordance with the invention to modulate expression of the subject polynucleotides in vivo. For example, antisense DNA molecules may be engineered and used to block translation of mRNA of the subject polynucleotides in vivo. Alternatively, ribozyme molecules may be designed to cleave and destroy the mRNA of the subject polynucleotides in vivo. In another alternative, oligonucleotides designed to hybridize to the 5′ region of the subject polynucleotide (including the region upstream of the coding sequence) and form triple helix structures may be used to block or reduce transcription of the subject polynucleotide. In yet another alternative, nucleic acid encoding the full length wild-type subject polynucleotide may be introduced in vivo into cells which otherwise would be unable to produce the wild-type subject polynucleotide product in sufficient quantities or at all.

For example, in conventional replacement therapy, gene product or its functional equivalent is provided to the patient in therapeutically effective amounts. Fat regulated gene protein can be purified using conventional techniques such as those described in Deutcher M. (ed.), 1990, Guide to Protein Petrification. Meth. Enzymol., Vol. 182. Sufficient amounts of gene product or protein for treatment can be obtained, for example, through cultured cell systems or synthetic manufacture. Drug therapies which stimulate or replace the gene product can also be employed. Delivery vehicles and schemes can be specifically tailored to the particular target gene.

Gene therapy using recombinant technology to deliver the gene into the patient's cells, or vectors which will supply the patient with gene product in vivo, is also within the scope of the invention. Retroviruses have been considered preferred vectors for experiments in somatic gene therapy, with a high efficiency of infection and stable integration and expression (Orkin et al., 1988, Prog. Med. Genet., 7: 130-142). For example, fat regulated gene cDNAs can be cloned into a retroviral vector and driven from either its endogenous promoter or from the retroviral LTR (long terminal repeat). Other delivery systems which can be utilized include adeno-associated virus (McLaughlin et al., 1988, J. Virol., 62: 1963-1973), vaccinia virus (Moss et al., 1987, Annu. Rev. Immunol., 5: 305-324), bovine papilloma virus (Rasmussen et al., 1987, Meth. Enzymol., 139: 642-654), or a member of the herpes virus group such as Epstein-Barr virus (Margolskee et al., 1988, Mol. Cell. Biol., 8: 2837-2847).

Antisense, ribozyme and triple helix nucleotides are designed to inhibit the translation or transcription of the subject polynucleotides. To accomplish this, the oligonucleotides used should be designed on the basis of relevant sequences unique to the subject polynucleotides. For example, and not by way of limitation, the oligonucleotides should not fall within those regions where the nucleotide sequence of a subject polynucleotide is most homologous to that of other polynucleotides, herein referred to as “unique regions”.

In the case of antisense molecules, it is preferred that the sequence be chosen from the unique regions. It is also preferred that the sequence be at least 18 nucleotides in length in order to achieve sufficiently strong annealing to the target mRNA sequence to prevent translation of the sequence (Izant J. G. and Weintraub H., 1984, Cell, 36: 1007-1015 and Rosenberg et al., 1985, Nature, 313: 703-706).

In the case of the “hammerhead” type of ribozymes, it is also preferred that the target sequences of the ribozymes be chosen from the unique regions. Ribozymes are RNA molecules which possess highly specific endoribonuclease activity. Hammerhead ribozymes comprise a hybridizing region which is complementary in nucleotide sequence to at least part of the target RNA, and a catalytic region which is adapted to cleave the target RNA. The hybridizing region contains 9 or more nucleotides. Therefore, the hammerhead ribozymes of have a hybridizing region which is complementary to the sequences listed above and is at least nine nucleotides in length. The construction and production of such ribozymes are well known in the art and are described more fully in Haseloff J. and Gerlach W. L., 1988, Nature, 334: 585-591.

The ribozymes also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug et al., 1984, Science, 224: 574-578; Zaug A. J. and Cech T. R, 1986, Science, 231: 470-475; Zaug et al., 1986, Nature, 324: 429-433; Patent Publication Treaty International Patent Application No. WO 88/04300 and Been M. D. and Cech T. R., 1986, Cell, 47: 207-216). The Cech endoribonucleases have an eight base pair active site which hybridizes to a target RNA sequence whereafter cleavage of the target RNA takes place. Cech-type ribozymes target eight base-pair active site sequences are present in a subject polynucleotide but not other polynucleotides for fat regulated proteins.

The compounds can be administered by a variety of methods which are known in the art, including, but not limited to the use of liposomes as a delivery vehicle. Naked DNA or RNA molecules may also be used where they are in a form which is resistant to degradation, such as by modification of the ends, by the formation of circular molecules, or by the use of alternate bonds including phosphothionate and thiophosphoryl modified bonds. In addition, the delivery of nucleic acid may be by facilitated transport where the nucleic acid molecules are conjugated to polylysine or transferrin. Nucleic acid may also be transported into cells by any of the various viral carriers, including but not limited to, retrovirus, vaccinia, adeno-associated virus, and adenovirus.

Alternatively, a recombinant nucleic acid molecule which encodes, or is, such antisense, ribozyme, triple helix, or subject polynucleotide molecule can be constructed. This nucleic acid molecule may be either RNA or DNA. If the nucleic acid encodes an RNA, it is preferred that the sequence be operatively attached to a regulatory element so that sufficient copies of the desired RNA product are produced. The regulatory element may permit either constitutive or regulated transcription of the sequence. A transfer vector such as a bacterial plasmid or viral RNA or DNA, encoding one or more of the RNAs, may be transfected into cells or cells of an organism (Llewellyn et al., 1987, J. Mol. Biol., 195: 115-123 and Hanahan et al., 1983, J. Mol. Biol., 166: 557-580). Once inside the cell, the transfer vector may replicate, and be transcribed by cellular polymerases to produce the RNA or it may be integrated into the genome of the host cell. Alternatively, a transfer vector containing sequences encoding one or more of the RNAs may be transfected into cells or introduced into cells by way of micromanipulation techniques such as microinjection, such that the transfer vector or a part thereof becomes integrated into the genome of the host cell.

Composition, Formulations, and Administration of Pharmaceutical Compositions

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol, or cellulose preparations such as, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges (e.g. gelatin) for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multidose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a co-solvent system comprising benzyl alcohol, a nonpolar surfactant, a water-miscible organic polymer, and an aqueous phase. Naturally, the proportions of a co-solvent system may be varied considerably without destroying its solubility and toxicity characteristics. Furthermore, the identity of the co-solvent components may be varied.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of therapeutic reagent, additional strategies for protein stabilization may be employed.

The pharmaceutical compositions also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the compounds of the invention may be provided as salts with pharmaceutically compatible counterions. Pharmaceutically compatible salts may be formed with many acids, including but, not limited to, hydrochloric, sulfuric, acetic, lactic, tartaric, malic, succinic, etc. Salts tend to be more soluble in aqueous or other protonic solvents than are the corresponding free base forms.

Suitable routes of administration may, for example, include oral, rectal, transmucosal, transdermal, or intestinal administration; or parenteral delivery, including intramuscular, subcutaneous, intramedullary injections, as well as intrathecal, direct intraventricular, intravenous, intraperitoneal, intranasal, or intraocular injections.

Alternately, one may administer the compound in a local rather than systemic manner, for example, via injection of the compound directly into an affected area, often in a depot or sustained release formulation.

Furthermore, one may administer the drug in a targeted drug delivery system, for example, in a liposome coated with an antibody specific for affected cells. The liposomes will be targeted to and taken up selectively by the cells.

The pharmaceutical compositions generally are administered in an amount effective for treatment or prophylaxis of a specific indication or indications. It is appreciated that optimum dosage will be determined by standard methods for each treatment modality and indication, taking into account the indication, its severity, route of administration, complicating conditions and the like. In therapy or as a prophylactic, the active agent may be administered to an individual as an injectable composition, for example, as a sterile aqueous dispersion, preferably isotonic. A therapeutically effective dose further refers to that amount of the compound sufficient to result in amelioration of symptoms associated with such disorders. Techniques for formulation and administration of the compounds of the instant application may be found in “Remington's Pharmaceutical Sciences,” Mack Publishing Co., Easton, Pa., latest edition. For administration to mammals, and particularly humans, it is expected that the daily dosage level of the active agent will be from 0.001 mg/kg to 10 mg/kg, typically around 0.01 mg/kg. The physician in any event will determine the actual dosage which will be most suitable for an individual and will vary with the age, weight and response of the particular individual. The above dosages are exemplary of the average case. There can, of course, be individual instances where higher or lower dosage ranges are merited, and such are within the scope of this invention.

The compounds of the invention may be particularly useful in animal disorders (veterinarian indications), and particularly mammals.

The invention further provides diagnostic and pharmaceutical packs and kits comprising one or more containers filled with one or more of the ingredients of the aforementioned compositions of the invention. Associated with such container(s) can be a notice in the form prescribed by a governmental agency regulating the manufacture, use or sale of pharmaceuticals or biological products, reflecting approval by the agency of the manufacture, use or sale of the product for human administration.

### EXAMPLES

The present invention is further described by the following examples. These examples, while illustrating certain specific aspects of the invention, do not portray the limitations or circumscribe the scope of the disclosed invention.

### Example 1

### Differential Gene Expression in Liver of Rats Fed Diets Supplemented with PUFAs

A. Animal Experimentation

Four week old female Wistar rats (52-72 g) were purchased from Charles River Canada Ltd. and housed 3 or 4 per cage for a period of 4 weeks. The rats were randomly divided into 6 groups and fed fat free diet (Teklad #94013). After 2 weeks, groups 2-6 were fed diets supplemented with highly purified polyunsaturated fatty acids for an additional 2 weeks. The concentration of the specific fatty acid used for each group was 5% (w/w) of the total diet preparation. The experimental design is provided below.

Group 1—Fat Free Controls

Group 2—Linoleic Acid (LA)—provided as safflower oil

Group 3—Alpha-Linolenic Acid (ALA)—provided as flax oil

Group 4—Gamma-Linolenic Acid (GLA)—provided as 1,3-GLA propane diol

Group 5—Eicosapentaenoic Acid (EPA)—provided as 1,3-EPA propane diol

Group 6—Docosahexaenoic Acid (DHA)—provided as 1,3-DHA propane diol

Diets were prepared by mixing the oils into the fat free diet. The mixed diet was stored at −20° C. under a nitrogen atmosphere to prevent fatty acid oxidation. Dietary feedings were provided at the same time each day. Diet preparations are shown below.

Group 2 (LA)—62.5 g safflower oil+937.5 g fat free diet

Group 3 (ALA)—87.7 g flax oil+912.3 g fat free diet

Group 4 (GLA)—55.8 g GLA diol+944.3 g fat free diet

Group 5 (EPA)—57.9 g EPA diol+942.1 g fat free diet

Group 6 (DHA)—59.7 g DHA diol+940.3 g fat free diet

Rats (166-2.29 g) were sacrificed via cardiac puncture under anesthesia. The livers were perfused with saline, excised and frozen in liquid nitrogen in preparation for RNA extraction.

B. RNA Isolation from Rat Liver and DNase Treatment

Total RNA from 0.4 g of each liver was isolated as per manufacturer's protocol using Trizol Reagent (Gibco BRL). In order to remove contaminating genomic DNA, the RNA sample was incubated with RQ1 RNase-free DNase (Promega) for 30 minutes at 37° C. The RNA was subsequently precipitated and resuspended to a final concentration of 0.5 mg/ml.

C. Reverse-Transcription of RNA to Single-Stranded cDNA

Reverse transcription of RNA to single-stranded cDNA was carried out in order to obtain template cDNA for differential display reactions. Two μg of total RNA was reverse-transcribed using M-MLV reverse transcriptase (Gibco BRL) using oligo(dT) primer in a total volume of 10 μl, following manufacturer's protocol.

D. Differential Display PCR

Rat liver cDNA was amplified using 90 combinations of 10 arbitrary P primers and 9 anchored T primers as well as 55 combinations of P primers only (Clontech, Delta Differential Display Kit). Control, GLA and DHA samples from three individual rats per treatment group were prepared for a total of nine samples for each primer pair. The differential display procedure was carried out following the manufacturer's protocol (Clontech) using Advantage 2 DNA polymerase (Clontech) and cDNA obtained from 55 ng of total RNA. Typically, reactions for five primer pairs were carried out at the same time using master-mixes whenever possible. These samples were then run on a 4.5 per cent polyacrylamide gel containing 7 M urea. The experiment was repeated to verify the results. Only bands which were differentially expressed in both experiments were further characterized.

The following primers (given in 5′ to 3′ direction) were used:

P primers
P1, ATTAACCCTCACTAAATGCTGGGGA;
P2, ATTAACCCTCACTAAATCGGTCATAG;
P3, ATTAACCCTCACTAAATGCTGGTGG;
P4, ATTAACCCTCACTAAATGCTGGTAG;
P5, ATTAACCCTCACTAAAGATCTGACTG;
P6, ATTAACCCTCACTAAATGCTGGGTG;
P7, ATTAACCCTCACTAAATGCTGTATG;
P8, ATTAACCCTCACTAAATGGAGCTGG;
P9, ATTAACCCTCACTAAATGTGGCAGG;
P10, ATTAACCCTCACTAAAGCACCGTCC.
T primers
T1, CATTATGCTGAGTGATATCTTTTTTTTTAA;
T2, CATTATGCTGAGTGATATCTTTTTTTTTAC;
T3, CATTATGCTGAGTGATATCTTTTTTTTTAG;
T4, CATTATGCTGAGTGATATCTTTTTTTTTCA;
T5, CATTATGCTGAGTGATATCTTTTTTTTTCC;
T6, CATTATGCTGAGTGATATCTTTTTTTTTCG;
T7, CATTATGCTGAGTGATATCTTTTTTTTTGA;
T8, CATTATGCTGAGTGATATCTTTTTTTTTGC;
T9, CATTATGCTGAGTGATATCTTTTTTTTTGG.

E. Cloning of Differentially Displayed Bands

Each differentially expressed cDNA fragment was excised from the gel and subsequently amplified using the same P- and T- or P- and P-primer combination which was initially used for the differential display procedure. The purified PCR fragments (Qiagen) were ligated (Boehringer Mannheim, Rapid DNA Ligation) into linearized pCRII vector (Invitrogen). Plasmid DNA from ten individual colonies was isolated (Qiagen) and their inserts sequenced on both strands using vector-specific fluorescence-labeled primer (Li-Cor), following manufacturer's specifications. Sequence analysis was performed using Vector NTI Suite (InforMax Inc.).

F. Northern Blot Analysis

To confirm the expression pattern of the genes as discovered in the differential display experiment and to determine the transcript size of these genes, Northern blot analysis was performed. Eight μg of total RNA from three different rats of each of the treatment groups 1, 2, 4, 5 and 6 (see above) was loaded onto a formaldehyde agarose gel, electrophoretically separated and transferred via capillary action to a nylon membrane (BioRad) according to Sambrook et al., 1989, Molecular Cloning, 2nd Edition, Cold Spring Harbor Laboratory Press, Cold Spring Harbour, N.Y. The transfer of the RNA onto the membrane was verified by methylene blue staining. The DNA probes for the Northern blots were obtained by amplifying the insert DNA from the plasmid harboring partial DNA sequences from differentially expressed genes using the two primers 5′-ACTAGCTAACGGCCGCCAGTGTGCTGG-3′and 5′-TGAGCTGGATATCTGCAGAATTCGGC-3′ which bind on both sides next to the insertion site of pCRIL Insert DNA was labeled using [alpha-³²P]dCTP and Ready-To-Go DNA Labeling Beads (Amersham Pharmacia Biotech) following manufacturer's specifications. The Northern blot was performed following the protocol in Ausubel et al., 1994-, Current Protocols in Molecular Biology, John Wiley & Sons, NY. The membrane was hybridized overnight at 42° C. and washed to a final stringency of 0.25×SSC, 0.1% SDS at 55° C. All membranes were probed with a nucleotide probe against the 18S rRNA subunit for normalization of loading.

G. Genes Identified by Differential Display

This experiment led to the identification of 14 genes with altered expression after PUFA treatment. The following 17 plasmids containing cDNA fragments which were obtained from this experiment represent the 14 unique genes.

TABLE 1

| | | Accession | DNA fragment^{‡} | | | |
|---|---|---|---|---|---|---|
| Plasmid | Gene | Number^{¶} | start | end | Total | Location* |
| pCr2001.1 | METP | | | | 889 | CDS + 3′ |
| pCr2002.1 | A1BG | | | | 440 | CDS + 3′ |
| pCr2003.1 | ApoA-1 | J02597 | 1040 | 1551 | 558 | CDS (444/1485) + 3′ |
| pCr2004.1 | COMT | Z12651 | 131 | 431 | 336 | CDS (301/795) |
| pCr2005.1 | INSIG1 | L13619 | 820 | 2363 | 1587 | 3′ |
| pCr2006.1 | Delta-3, delta-2-enoyl- | X61184 | −2 | 608 | 637 | 5′ + CDS (608/867) |
| | CoA isomerase | | | | | |
| pCr2007.1 | FAS | X62888 | 3215 | 3513 | 339 | CDS (361/7518) |
| pCr2008.1 | G6PD | X07467 | 156 | 1151 | 1030 | CDS (996/1548) |
| pCr2009.1 | G6PD | X07467 | 1124 | 2255 | 1165 | CDS (425/1548) + 3′ |
| pCr2010.1 | GPAT | M77003 | 4972 | 5747 | 797 | 3′ |
| pCr2011.1 | Spot-14 | K01934 | 250 | 1241 | 1038 | CDS (203/453) |
| pCr2012.1 | GLOL | D83198 | 35 | 827 | 838 | CDS (648/684) + 3′ |
| pCr2013.1 | FTF1 | AB017614 | 755 | 1706 | 981 | CDS (770/1524) + 3′ |
| pCr2014.1 | SCD1 | J02585 | 2175 | 2677 | 538 | 3′ |
| pCr2015.1 | SCD1 | J02585 | 3960 | 4362 | 439 | 3′ |
| pCr2016.1 | SCD2 | AB032243 | −235 | 995 | 1276 | 5′ + CDS (995/1077) |
| pCr2017.1 | SCD2 | AB032243 | 3915 | 4719 | 836 | 3′ |

¶M77003 and AB017614 refer to mouse and D83198 refers to human nucleotide sequences. All other accession numbers refer to rat genes.
‡start and end mean the beginning and the end of the gene-specific insert counting from the translation start-codon (A of AUG designated as 1), total is the number of all nucleotides of the insert including the two non-gene specific primers.
*e.g. ApoA-1 CDS (444/1485) + 3′: plasmid contains 444 nucleotides of the total CDS (1485 nucleotides) and 114 nucleotides of the 3′ UTR.

TABLE 2
The table lists the values of the quantification of the Northern
blots in percent ± standard deviation of three individual
rats per treatment group. All membranes were probed with a
nucleotide probe against the 18S rRNA subunit and all values
are normalized for 18S except for G6PD, A1BG and FTF1.
However, the loading differences between the different
lanes on the gels are usually less than 15%.

| | fat free | LA | GLA | EPA | DHA |
|---|---|---|---|---|---|
| SCD1 | 100 ± 17 | 63 ± 20 | 6 ± 8 | N.D. | 1 ± 1 |
| SCD2 | 100 ± 23 | 63 ± 15 | 11 ± 6 | 2 ± 1 | 4 ± 1 |
| G6PD | 100 ± 11 | 42 ± 27 | 8 ± 9 | 15 ± 14 | 19 ± 7 |
| GPAT | 100 ± 35 | 52 ± 5 | 15 ± 13 | 14 ± 10 | 26 ± 15 |
| FAS | 100 ± 33 | 47 ± 40 | 17 ± 12 | 17 ± 14 | 27 ± 21 |
| delta-3, delta-2- | 14 ± 18 | 29 ± 27 | 33 ± 15 | 100 ± 35 | 87 ± 2 |
| enoyl-CoA | | | | | |
| isomerase | | | | | |
| COMT | 100 ± 32 | 59 ± 6 | 45 ± 23 | 50 ± 4 | 49 ± 3 |
| ApoA-1 | 100 ± 24 | 90 ± 23 | 32 ± 8 | 25 ± 4 | 12 ± 1 |
| Spot-14 | 100 ± 16 | 50 ± 6 | 12 ± 7 | 19 ± 25 | 24 ± 18 |
| INSIG1 | 100 ± 44 | 49 ± 12 | 11 ± 2 | 25 ± 3 | 13 ± 11 |
| FTF1 | 100 ± 16 | 56 ± 15 | 4 ± 8 | 10 ± 7 | 13 ± 12 |
| GLOL | 100 ± 17 | 64 ± 6 | 37 ± 11 | 29 ± 15 | 36 ± 17 |
| METP | 10 ± 5 | 37 ± 7 | 78 ± 12 | 100 ± 16 | 80 ± 28 |
| A1BG | 7 ± 9 | 52 ± 47 | 14 ± 10 | 32 ± 30 | 100 ± 64 |

(N.D. indicates “not detected”).

### Example 2

### Cloning of rMETP and hMETP into Mammalian Expression Vector

A. Obtaining 5′ End of rMETP

Clones containing the complete coding sequence for rMETP were obtained from the Superscript rat liver cDNA library (Gibco BRL) using the Genetrapper cDNA Positive Selection System (Gibco BRL) as per the manufacturer's instructions. The sequence of the oligonucleotide used to probe the library and repair the captured cDNA target was 5′-GTGGTCTTCGTGGCTTATGAG-3′. The repaired DNA was used to transform UltraMax DH5α-FT cells (Gibco BRL). Colonies containing rMETP were identified by colony-PCR using 5′-GCTACGCCCATGGACGTTATCAAGTCC-3′ and 5′-CAGGCAGTATGGCATTTGA-3′ as gene specific primers. PCR reactions contained 0.5 μM of each primer, 200 μM of each dATP, dCTP, dGTP and dTTP, 2 mM MgCl₂, 1× PCR Buffer (Gibco BRL) and 0.025 U/μl of Platinum Taq polymerase (Gibco BRL). The PCR conditions were: 95° C. for 2 min; 35 cycles of 98° C. for 30 sec, 58° C. for 45 sec and 72° C. for 45 sec; 72° C. for 7 min. Plasmid DNA was isolated from selected colonies and sequenced. The sequence obtained was used to identify the open reading frame for rMETP and design primers for subcloning the gene. A plasmid containing the complete coding sequence was designated pSr1066.1.

B. Cloning of METP

Rat and human METP were cloned by PCR into the pcDNA3.1/Myc-His(+) mammalian expression vector (Invitrogen). For each gene two plasmid constructions were made for the production of the METP protein with a C-terminal tag containing the c-myc epitope and polyhistidine peptide (i.e., rMETP/Myc-His and hMETP/Myc-His), or the METP protein without the tag (i.e., rMETP and hMETP). The forward primers for cloning the rat and human genes were 5′-CACGCGGGATCCGCCACCATGGATTTTGTTGCTGGGGCCATTGG-3′and 5′-CACGCGGGTACCACGCGTGCCACCATGGATTTTGTCGCTGGAGCCATCG-3′, respectively. These primers contain the Kozak consensus sequence adjacent to the translation start codon. The primer for the rat gene contains a BamHI site (underlined) whereas the primer for the human gene contains a KpnI site (underlined). The sequences of the reverse primers for cloning rMETP and hMETP were 5′-ATATCACGATGCGGCCGCCTATGTGAGCAGGCCCTGAGTGAGCC-3′and 5′-ATATCACGATGCGGCCGCCTATGTGAGCAGACCCCGGGCGAGC-3′, respectively. These primers contain a NotI site (underlined) and provide the translation stop codon. The reverse primers for cloning rMETP/Myc-His and hMETP/Myc-His were 5′-ATATCAGGATGCGGCCGCCATGTGAGCAGGCCCTGAGTGAGCC-3′ and 5′-ATATCACGATGCGGCCGCCATGTGAGCAGACCCCGGGCGAGC-3′, respectively. These primers contain a NotI site (underlined) with only 2 of the 3 bases of the stop codon, therefore, placing the gene in frame with the tag provided by the vector.

PCR was carried out using Advantage-HF polymerase (Clontech) as per the manufacturer's instructions. pSr1066.1 was used as the DNA template for generating rMETP/Myc-His. pMr1076.1 (described below) was used as the DNA template for rMETP. Plasmid DNA from the Proquest human liver cDNA library (Gibco BRL) was used as the template for the human gene.

The PCR products were gel purified, digested with NotI and either BamHI (rat gene) or KpnI (human gene), and ligated into pcDNA3.1/Myc-His(+)A cut with the corresponding enzymes. The ligation products were used to transform E. coli strain TOP10 (Invitrogen). Plasmids were isolated and sequenced. Plasmids for rMETP/Myc-His and rMETP were designated pMr1076.1 and pMr1076.2, respectively. For both hMETP/Myc-His and hMETP two different sequences were obtained. About half of the isolates had a G at position 404 of the coding sequence (Arg¹³⁵) and half had a T at this position (Leu¹³⁵) indicating a possible site for genetic polymorphism (which may be useful in screening for genetic characteristics). Plasmids for hMETP/Myc-His and hMETP containing a G at position 404 were designated pMh1081.1 and pMh1084.1, respectively. Plasmids for hMETP/Myc-His and hMETP containing a T at position 404 were designated pMh1083.1 and pMh105.1, respectively.

### Example 3

### Cloning of rGLOL and hGLOL into Mammalian Expression Vector

A. Obtaining 5′ End of rGLOL

The 5′ end of rGLOL cDNA was cloned by PCR. The sequence of the forward primer (5′-CCGGTCCTCCTGCGGCAGATG-3′) was based on a mouse EST (GenBank Accession No. AW106717) which codes for the 5′ end of the mouse ortholog of rGLOL. The primer corresponds to 5′ UTR and the translation start codon (underlined). The reverse primer (5′-TGGAAGTCTGTTCGTCCACA-3′) was based on the rGLOL clone obtained by differential display. PCR was carried out with Platinum Taq polymerase (Gibco BRL) as previously described (Example 2). Plasmid DNA isolated from the Superscript rat liver cDNA library (Gibco BRL) was used as the template. The PCR product was gel purified and inserted by TA cloning into the pCRII vector (Invitrogen). The ligation products were used to transform E. coli strain TOP10 (Invitrogen). Plasmid DNA was isolated and sequenced. The sequence obtained was used to design a forward primer for cloning the gene. A plasmid containing the 59 end of rGLOL was designated pCr1067.1.

B. Cloning of GLOL

Rat and human GLOL were cloned by PCR into the pcDNA3.1/Myc-His(+) mammalian expression vector (Invitrogen). For each gene two plasmid constructions were made for the production of the GLOL protein with a C-terminal tag containing the c-myc epitope and polyhistidine peptide (i.e., rGLOL/Myc-His and hGLOL/Myc-His), or the GLOL protein without the tag (i.e., rGLOL and hGLOL). The forward primers for cloning the rat and human genes were 5′-CACGCGGGATCCGCCACCATGTTTGAGCCCAAGAGCTGCACC-3′ and 5′-CACGCGGGATCCGCCACCATGTTCGAGCCTGTGAGCTGCACC-3′, respectively. These primers contain the Kozak consensus sequence adjacent to the translation start codon and a BamHI site (underlined). The sequences of the reverse primers for cloning rGLOL and hGLOL were 5′-ATATCACGATGCGGCCGCTCAGGAGGGTGGAGTCTGGACCC-3′ and 5′-ATATCACGATGCGGCCGCTCAGGCAGTGGGTGTCTGCACC -3′, respectively. These primers contain a NotI site (underlined) and provide the translation stop codon. The reverse primers for cloning rGLOL/Myc-His and hGLOL/Myc-His were 5′-ATATCACGATGCGGCCGCCAGGAGGGTGGAGTCTGGACCC-3′ and 5′-ATATCACGATGCGGCCGCCAGGCAGTGGGTGTCTGCACC-3′, respectively. These primers contain a NotI site (underlined) with only 2 of the 3 bases required for the stop codon, therefore, placing the gene in frame with the tag provided by the vector.

PCR was carried out using Advantage-HF polymerase (Clontech) as per the manufacturer's instructions. Plasmid DNA from the Superscript rat liver cDNA library (Gibco BRL) was used as the DNA template for generating rGLOL/Myc-His. pMr1075.1 (described below) was used as the DNA template for rGLOL. Plasmid DNA from the Proquest human liver cDNA library (Gibco BRL) was used as the template for generating hGLOL/Myc-His. pMr1072.1 (described below) was used as the DNA template for hGLOL.

The PCR products were gel purified, digested with BamHI and NotI, and ligated into pcDNA3.1/Myc-HIS(+)A cut with the same enzymes. The ligation products were used to transform E. coli strain TOP10 (Invitrogen). Plasmids were isolated and sequenced. Plasmids for rGLOL/Myc-His and rGLOL were designated pMr1075.1 and pMr1075.2, respectively. Plasmids for hGLOL/Myc-His and hGLOL were designated pMh1072.1 and pMh1072.2, respectively. The sequence obtained for the human gene, which was confirmed by cloning and sequencing products from independent PCR reactions, contained 1 base different than the sequence reported in GenBank Accession No. D83198 with an A instead of a G at position 46 of the coding sequence. This results in Arg¹⁶ instead of Gly¹⁶.

### Example 4

### Cloning of rFTF1 and hFTF1 into Mammalian Expression Vector

A. Obtaining 5′ End of rFTF1

The 5′ end of rFTF1 cDNA was obtained using the SMART RACE cDNA amplification kit (Clontech) as per the manufacturer's instructions. The 5′-RACE-ready cDNA was prepared using Superscript II reverse transcriptase (Gibcb BRL) and 1 μg of total adipose RNA isolated from a female Wistar rat using Trizol Reagent (Gibco BRL). The 5′-RACE PCR reaction was set-up as recommended except it contained 1× Advantage-GC cDNA polymerase (Clontech), the corresponding buffer and 0.5 M GC-melt (Clontech). The sequence of the gene specific primer was 5′-CTGGAGCAGGGTCCTGTTGGCAGTCTCT-3′. The PCR conditions were: 94° C. for 3 min; 5 cycles of 94° C. for 30 sec, 69° C. for 10 sec and 72° C. for 3 min; 5 cycles of 94° C. for 30 sec. 67° C. for 10 sec and 72° C. for 3 min; 35 cycles of 94° C. for 30 sec, 65° C. for 10 sec and 72° C. for 3 min. The product was gel purified and inserted by TA cloning into the pCRII vector (Invitrogen). The ligation products were used to transform E. coli Top10 (Invitrogen). Plasmid DNA was isolated and sequenced. The sequence obtained was used to identify the open reading frame for rFTF1 and design a forward primer for cloning the gene into a mammalian expression vector. A plasmid containing the 5′-RACE product for rFTF1 was designated pCr1092.1.

B. Cloning of FTF1

Rat and human FTF1 were cloned by PCR into the pcDNA3.1/Myc-His(+) mammalian expression vector (Invitrogen). For each gene two plasmid constructions were made for the production of the FTF1 protein with a C-terminal tag containing the c-myc epitope and polyhistidine peptide (i.e., rFTF1/Myc-His and hFTF1/Myc-His), or the FTF1 protein without the tag (i.e., rFTF1 and hFTF1). The forward primers for cloning the rat and human genes were 5′-CACGCGGGTACCACGCGTGCCACCATGGACGCCGTCTTGGAACCTTTCC-3′ and 5′-CACGCGGGTACCACGCGTGCCACCATGGACGCCGTCTTGGAACCCTTCC-3′, respectively. These primers contain the Kozak consensus sequence adjacent to the translation start codon and a KpnI site (underlined). The sequence of the reverse primer for cloning rFTF1 and hFTF1 was 5′-CACGCGCTCGAGCTAGGAGAGTTTGATGGTGGTGTTGGG-3′. This primer contains a XhoI site (underlined) and provides the translation stop codon. The reverse primer for cloning rFTF1/Myc-His and hFTF1/Myc-His was 5′-CACGCGCTCGAGGGAGAGTTTGATGGTGGTGTTGGG-3′. This primer contains a XhoI site (underlined) but not the stop codon, therefore, placing the gene in frame with the tag provided by the vector.

PCR was carried out using Advantage-GC cDNA polymerase and 1 M GC-melt (Clontech) as per the manufacturer's instructions. Plasmid DNA from the Superscript rat liver cDNA library (Gibco BRL) was used as a template for the rat gene. Plasmid DNA from the Proquest human liver cDNA library (Gibco BRL) was used as the template for the human gene.

The PCR products were gel purified, digested with KpnI and XhoI, and ligated into pcDNA3.1/Myc-His(+)A cut with the same enzymes. The ligation products were used to transform E. coli strain TOP10 (Invitrogen). Plasmids were isolated and sequenced. The use of the non-high fidelity Advantage-GC cDNA polymerase resulted in several nucleotide substitutions. DNA fragments from several isolated clones were ligated resulting in a polynucleotide sequence coding for the correct polypeptide sequence. Plasmids for rFTF1/Myc-His and rFTF1 were designated pMr1088.1 and pMr1089.1, respectively. Plasmids for hFTF1/Myc-His and hFTF1 were designated pMh1090.1 and pMh1091.1, respectively.

### Example 5

### Cloning of rA1BG an hA1BG into Mammalian Expression Vector

A. Obtaining 5′ End of rA1BG

The 5′ end of the rA1BG gene was obtained by using the SMART RACE cDNA amplification kit (Clontech) as per the manufacturer's instructions. The 5′-RACE-ready cDNA was prepared using Superscript II reverse transcriptase (Gibco BRL) and 1 μg of total hepatic RNA isolated from a rat fed the DHA-supplemented diet used in the differential display study. The 5′-RACE PCR reaction was carried out as recommended by the manufacturer except it contained 1× Advantage-HF polymerase mix (Clontech) and the corresponding buffer. The sequence of the gene specific primer was 5′-CAACCCTTTCCAAGGGCACTTTCCTGTG-3′. The product was gel purified and ligated by TA cloning into the pCRII vector (Invitrogen). The ligation products were used to transform E. coli strain TOP10 (Invitrogen). Plasmid DNA was isolated and sequenced. The sequence obtained was used to identify the open reading frame for rA1BG and design primers for cloning the gene into a mammalian expression vector. A plasmid containing the 5′-RACE product for rA1BG was designated pCr1078.1.

B. Cloning of A1BG

Rat and human A1BG were cloned by PCR into the pcDNA3.1/Myc-His(+) mammalian expression vector (Invitrogen). For each gene two plasmid constructions were made for the production of the A1BG protein with a C-terminal tag containing the c-myc epitope and polyhistidine peptide (ie. rA1BG/Myc-His and hA1BG/Myc-His), or the A1BG protein without the tag (ie. rA1BG and hA1BG). The forward primers for cloning the rat and human genes were 5′-CACGCGGGATCCGCCACCATGTCTCTGTTGACTACTG-3′ and 5′-CACGCGGGATCCGCCACCATGTCCATGCTCGTGGTTTCTC-3′, respectively. These primers contain the Kozak consensus sequence adjacent to the translation start codon and a BamHI site (underlined). The sequences of the reverse primers for cloning rA1BG and hA1BG were 5′-ATATCACGATGCGGCCGCTTAGCTACCTTCTACTACAACTTCCACAGGG-3′ and 5′-ATATCACGATGCGGCCGCTCAGCTTTCTGCCACCAGGAGC-3′, respectively. These reverse primers contain a NotI site (underlined) and provide the translation stop codon. The reverse primers for cloning rA1BG/Myc-His and hA1BG/Myc-His were 5′-ATATCACGATGCGGCCGCTTGCTACCTFCTACTACAACTTCCACAGGG-3′ and 5′-ATATCACGATGCGGCCGCCAGCTTTCTGCCACCAGGAGC-3′, respectively. These primers contain a NotI site (underlined) with only 2 of the 3 bases required for the stop codon, therefore, placing the gene in frame with the tag provided by the vector.

PCR was carried out using Advantage-HF polymerase (Clontech) as per the manufacturer's instructions. The 5′-RACE-ready cDNA used to obtain the 5′ end of rA1BG was used as the DNA template for the rat gene. Plasmid DNA from the Proquest human liver cDNA library (Gibco BRL) was used as the template for the human gene.

The PCR products were gel purified, digested with BamHI and NotI, and ligated into pcDNA3.1/Myc-His(+)A cut with the same enzymes. The ligation products were used to transform E. coli strain TOP10 (Invitrogen). Plasmids were isolated and sequenced. Plasmids for rA1BG/Myc-His and rA1BG were designated pMr1082.1 and pMr1077.1, respectively. Plasmids for hA1BG/Myc-His and hA1BG were designated pMh1087.1 and pMh1086.1, respectively.

### Example 6

### Cloning of hGPAT into Mammalian Expression Vector

Human GPAT is cloned by PCR into the pcDNA3.1/Myc-His(+) mammalian expression vector (Invitrogen). The forward primer, 5′-CACGCGGGATCCGCCACCATGGATGAATCTGCACTGACCCTTGG-3′, contains the Kozak consensus sequence adjacent to the translation start codon and a BamHI site (underlined). The reverse primer, 5′-ATATCACGATGCGGCCGCCTACAGCACCACAAAACTCAGAATATATTCTAG-3′, contains a NotI site (underlined) and provides the translation stop codon.

PCR is carried out using Advantage-HF 2 polymerase (Clontech) as per the manufacturer's instructions and plasmid DNA from the Proquest human liver cDNA library (Gibco BRL).

The PCR products are gel purified, digested with BamHI and NotI, and ligated into pcDNA3.1/Myc-His(+)A cut with the same enzymes. The ligation products are used to transform E. coli strain TOP10 (Invitrogen). Plasmids are isolated and sequenced.

### Example 7

### Cloning of the Human SCD Control Region

The human SCD promoter was cloned from human leukocyte genomic DNA. Blood was obtained from volunteers in the present inventors' laboratory and used to prepare genomic DNA. The control region corresponding to positions-1981 bp to -12 bp upstream of the ATG was amplified using synthetic forward and reverse primers. To facilitate cloning into the plasmid pGL3-basic (Promega), the recognition sequence for BglII restriction enzyme (which is absent in the sequence to be amplified) was introduced at the ends of the forward and reverse primers.

The forward and reverse primers used for cloning the human SCD control region by PCR amplification are 5′-GGAAGATCTGAGAGCGAGACTTCCTCTCAA-3′ and 5′-GGAAGATCTGATGCCGGGATCACTTTCC-3′, respectively.

The PCR amplification was conducted in a Perkin-Elmer GeneAMP PCR system 9700 instrument, in a 50 μl reaction volume containing: 0.5 μg of genomic DNA, 0.4 μM of each primer, 1× dNTP mix (Clontech, Calif.), 1× cDNA PCR reaction buffer (Clontech) and 1× Advantage cDNA polymerase mix (Clontech).

The conditions for the PCR reaction were:

7 cycles at 94° C. for 2 seconds, 72° C. for 3 minutes

32 cycles at 94° C. for 2 seconds, 67° C. for 3 minutes

67° C. for 4 minutes

The PCR product was gel-purified using QIAquick gel extraction kit (Qiagen, Germany). The purified PCR product and the reporter vector pGL3-basic were separately digested with BglII restriction enzyme to generate compatible ends suitable for in-frame ligation of the PCR product to the luciferase gene of pGL3-basic. The ligation product was used to transform E. coli TOP10 strain (Invitrogen). The resulting plasmid, pGh3022.1, was screened by restriction analysis and confirmed by DNA sequencing. The resulting human SCD control region/reporter construct is used to transfect different mammalian cell lines, and reporter activity measured.

### Example 8

### Cloning Human METP Control Region

The METP control region (1500 bp) is cloned from human leukocyte genomic DNA by PCR. The control region is amplified by PCR using synthetic forward and reverse primers starting at positions-1500 bp and -1 bp upstream the ATG. The forward and reverse primers used for cloning human METP control region by PCR amplification is 5′-TGGCCTTGGATGGGCCACTTCCCGC-3′ and 5′-GAACAAGGTGTGGCGGGGAGGCCCTGGGTC-3′, respectively.

The PCR amplification is conducted in a Perkin-Elmer GeneAMP PCR system 9700 instrument. For example, the PCR is performed in a 50 μl reaction volume containing: 0.5 μg of genomic DNA, 0.4 μM of each primer, 1× dNTP mix (Clontech, Calif.), 1× cDNA PCR reaction buffer (Clontech) and 1× Advantage cDNA polymerase mix (Clontech).

The conditions for the PCR reaction are:

7 cycles at 94° C. for 2 seconds, 72° C. for 3 minutes

32 cycles at 94° C. for 2 seconds, 67° C. for 3 minutes

67° C. for 4 minutes

The PCR product is gel-purified using QIAquick gel extraction kit (Qiagen, Germany) and ligated into the TA cloning vector pCRII (Invitrogen) according to manufacturers instruction. The ligation product is used to transform E. coli TOP10 strain (invitrogen). The resulting plasmids are screened by restriction analysis and confirmed by DNA sequencing. The human METP control region is then recloned from the pCRII vector into the luciferase reporter vector pGL3-Basic (Promega). The resulting human METP control region/reporter construct is used to transfect different mammalian cell lines, and reporter activity measured.

### Example 9

### Cloning Human GLOL Control Region

The GLOL control region (468 bp, spanning positions-905 to 438) is cloned from human leukocyte genomic DNA by PCR. The first 30 nucleotides of the 5′ end of GLOL control region is rich in A nucleotide (see FIG. 13), and could make cloning by PCR difficult if incorporated in primers. Therefore, the control region is amplified by PCR using synthetic forward and reverse primers starting at positions-886 bp and 438 bp upstream the ATG. The forward and reverse primers used for cloning human GLOL control region by PCR amplification is 5′-GGAAAAAAAACGGCCCCGAGGCTATGAGTG-3′ and 5′-CTGCCGCAGGAGGATGGGGGCTC-3′, respectively.

The PCR amplification is conducted in a Perkin-Elmer GeneAMP PCR system 9700 instrument. For example, the PCR is performed in a 50 μl reaction volume containing: 0.5 μg of genomic DNA, 0.4 μM of each primer, 1× dNTP mix (Clontech, Calif.), 1× cDNA PCR reaction buffer (Clontech) and 1× Advantage cDNA polymerase mix (Clontech).

The conditions for the PCR reaction are:

7 cycles at 94° C. for 2 seconds, 72° C. for 3 minutes

2 cycles at 94° C. for 2 seconds, 67° C. for 3 minutes

67° C. for 4 minutes

The PCR product is gel-purified using QIAquick gel extraction kit (Qiagen, Germany), and ligated into the TA cloning vector pCRII (Invitrogen) according to manufacturer's instruction. The ligation product is used to transform E. coli TOP10 strain (Invitrogen). The resulting plasmids are screened by restriction analysis and confirmed by DNA sequencing. The human GLOL control region is then recloned from the pCRII vector into the luciferase reporter vector pGL3-Basic (Promega). The resulting human GLOL control region/reporter construct is used to transfect different mammalian cell lines, and reporter activity measured.

### Example 10

### Cloning Human FTF1 Control Region

The FTF1 control region (883 bp, spanning position-883 to -1 upstream of the ATG) is cloned from human leukocyte genomic DNA by PCR. The first 30 nucleotides of the 5′ end of FTF1 control region is rich in A and T nucleotides (see FIG. 19), and could make cloning by PCR difficult if incorporated in primers. Therefore, the control region is amplified by PCR using synthetic forward and reverse primers starting at positions-850 bp and -4 bp upstream of the ATG respectively. The forward and reverse primers used for cloning human FTF1 control region by PCR amplification are 5′-TTAAAAAGCTGAAAATCTCCCCCGTTGAGGGGAGATC-3′ and 5′-CGCAGCCGGCTCCCGGGACCCCTTCC-3′, respectively.

The PCR amplification is conducted in a Perkin-Elmer GeneAMP PCR system 9700 instrument. For example, the PCR is performed in a 50 μl reaction volume containing: 0.5 μg of genomic DNA, 0.4 μM of each primer, 1× dNTP mix (Clontech, Calif.), 1× cDNA PCR reaction buffer (Clontech) and 1× Advantage cDNA polymerase mix (Clontech).

The conditions for the PCR reaction are:

7 cycles at 94° C. for 2 seconds, 72° C. for 3 minutes

32 cycles at 94° C. for 2 seconds, 67° C. for 3 minutes

67° C. for 4 minutes

The PCR product is gel-purified using QIAquick gel extraction kit (Qiagen, Germany), and ligated into the TA cloning vector pCRII (Invitrogen) according to manufacturers instruction. The ligation product is used to transform E. coli TOP10 strain (Invitrogen). The resulting plasmids are screened by restriction analysis and confirmed by DNA sequencing. The human FTF1 control region is then recloned from the pCRII vector into the luciferase reporter vector pGL3-Basic (Promega). The resulting human FTF1 control region/reporter construct is used to transfect different mammalian cell lines, and reporter activity measured.

### Example 11

### Cloning Human A1BG Control Region

The A1BG control region (431 bp) is cloned from human leukocyte genomic DNA by PCR. The control region is amplified by PCR using synthetic forward and reverse primers starting at positions-430 bp and -1 bp upstream the ATG. The forward and reverse primers used for cloning human A1BG control region by PCR amplification are 5′-TGAACCCCACCTTTGGTGTCACATGTGCAG-3′ and 5′-GATGGTCGCGCTCACTCCGGTGCAGTGAG-3′, respectively.

The PCR amplification is conducted in a Perkin-Elmer GeneAMP PCR system 9700 instrument. For example, the PCR is performed in a 50 μl reaction volume containing: 0.5 μg of genomic DNA, 0.4 μM of each primer, 1× dNTP mix (Clontech, Calif.), 1× cDNA PCR reaction buffer (Clontech) and 1× Advantage cDNA polymerase mix (Clontech).

The conditions for the PCR reaction are:

7 cycles at 94° C. for 2 seconds, 72° C. for 3 minutes

32 cycles at 94° C. for 2 seconds, 67° C. for 3 minutes

67° C. for 4 minutes

The PCR product is gel-purified using QIAquick gel extraction kit (Qiagen, Germany), and ligated into the TA cloning vector pCRII (Invitrogen) according to manufacturers instruction. The ligation product is used to transform E. coli TOP10 strain (Invitrogen). The resulting plasmids are screened by restriction analysis and confirmed by DNA sequencing. The human A1BG control region is then recloned from the pCRII vector into the luciferase reporter vector pGL3-Basic (Promega). The resulting human A1BG control region/reporter construct is used and to transfect different mammalian cell lines, and reporter activity measured.

### Example 12

### Cloning Human GPAT Control Region

The control region (1962 bp) is cloned from human leukocyte genomic DNA by PCR. The control region is amplified by PCR using synthetic forward and reverse primers starting at positions-34534 bp and -32575 bp upstream the ATG. The forward and reverse primers used for cloning human GPAT ccontrol region by PCR amplification is 5 ′-TTGGCTCACCTCAGTGCCCCCAGTC-3′ and 5′-TGGTTTTGCATCGTATCTTCCCCTCTGCTGCCATC-3′, respectively.

The PCR amplification is conducted in a Perkin-Elmer GeneAMP PCR system 9700 instrument. For example, the PCR is performed in a 50 μl reaction volume containing: 0.5 μg of genomic DNA, 0.4 μM of each primer, 1× dNTP mix (Clontech, Calif.), 1× cDNA PCR reaction buffer (Clontech) and 1× Advantage cDNA polymerase mix (Clontech).

The conditions for the PCR reaction are:

7 cycles at 94° C. for 2 seconds, 72° C. for 3 minutes

32 cycles at 94° C. for 2 seconds, 67° C. for 3 minutes

67° C. for 4 minutes

The PCR product is gel-purified using QIAquick gel extraction kit (Qiagen, Germany) and ligated into the TA cloning vector pCRII (Invitrogen) according to manufacturer's instruction. The ligation product is used to transform E. coli TOP10 strain (Invitrogen). The resulting plasmids are screened by restriction analysis and confirmed by DNA sequencing. The human GPAT control region is then recloned from the pCRII vector into the luciferase reporter vector pGL3-Basic (Promega). The resulting human GPAT control region/reporter construct is used to transfect different mammalian cell lines, and reporter activity measured.

### Example 13

### Drug Screening Assay Using Yeast Whole Cells or Spheroplasts

A. Spheroplast Preparation

Cultures of OLE1 deletion mutant of Saccharomyces cerevisiae (or other) transformed with human SCD gene with/without 6×His tag are started from a stock cell suspension. Yeast are grown in appropriate medium and 2% galactose to induce the expression of the gene that encodes the fatty acid delta-9-desaturase. After 16 h incubation, cells are centrifuged at 2060×g for 5 min at 4° C., washed once with distilled water and centrifuged. The volume and weight of the cell pellet is measured. Cells are suspended (1:2 w/v) in 0.1 M Tris-SO₄ (pH 9.4), 10 mM DTT and incubated at 30° C. After 10 min incubation, the cell pellet is obtained by centrifugation, washed once (1:20 w/v) with 1.2 M sorbitol and suspended (1:1 w/v) in 1.2 M sorbitol, 20 mM phosphate buffer (pH 7.4). The 15,800×g (1 min) supernatant of Lyticase is added to the cell suspension at a concentration of 2000 U/ml and incubated at 30° C. in an orbital shaker at 50 rpm. Conversion to spheroplasts is checked after 40 min incubation by diluting the suspension with distilled water followed by observation under the microscope. After 70 min incubation, approximately 90% of the cells are converted to spheroplasts.

B. Incubation of Spheroplasts with Test Compounds

Spheroplasts are harvested by centrifugation at 2060×g for 5 min at 4° C., washed once with 1.2 M sorbitol and resuspended in appropriate medium with 1% Brij 58, 1.2 M sorbitol and 2% galactose to maintain the induction conditions and to give an O.D.₆₀₀ reading of approximately 2.5-3.0. A 10 ml aliquot of the spheroplast suspension is transferred to several 125 ml Erlenmeyer flask and incubated with 200 μL of different concentrations of test compounds in each flask at 30° C. in an orbital incubator at 270 rpm. After 30 min incubation, 2-200 μM (1 μCi) of [1-¹⁴C]palmitic acid is added to the cultures and further incubated for 120 min. At this time point, O.D.₆₀₀ is determined, spheroplasts are harvested by centrifugation and washed with Tris-HCl buffer (100 mM, pH 8.0) containing 0.1% BSA.

C. Incubation of Whole Yeast Cells with Test Compounds

OLE1 deletion mutants of Saccharomyces cerevisiae transformed with human SCD gene are incubated in several 125 ml Erlenmeyer flask containing 9 ml of an appropriate medium with 1% Brij 58, (O.D.₆₀₀ 0.4, approximately 3.2×10⁶ cells/ml) and 200 μl of different concentrations of test compounds. After 1 h incubation at 30° C. in an orbital shaker at 270 rpm, 1 μCi of [1-¹⁴C]palmitic acid (dissolved in incubation medium with 1% Brij 58), is added to the cell suspension to a final concentration of 2-200 μM. After 5 h incubation with the inhibitor and radiolabelled fatty acid, the transgene expression is induced with the addition of galactose to a final concentration of 2%. Yeast are further incubated for 19 h until they are harvested by centrifugation at 5000×g for 10 minutes at 4° C. Cells are washed with Tris-HCl buffer (100 mM, pH 8.0) containing 0.1% BSA and total lipids are extracted as described below.

D. Lipid Analysis

Whole cells or spheroplast pellets are saponified with 10% KOH to obtain free fatty acids which are further methylated using BF₃ in methanol. The fatty acid methyl esters are analyzed by high performance liquid chromatography (HPLC) using a Hewlett Packard 1090, Series II chromatograph equipped with a diode array detector set at 205 nm, a radioisotope detector (Model 171, Beckman, Calif.) with a solid scintillation cartridge (97% efficiency for ¹⁴C-detection) and a reverse-phase ODS (C-18) Beckman column (250 mm×4.6 mm i.d.; 5 μm particle size) attached to a pre-column with a μBondapak C-18 (Beckman) insert. Fatty acid methyl esters are separated isocratically with acetonitrile/water (95:5 v:v) at a flow rate of I ml/min and are identified by comparison with authentic standards.

Alternatively, fatty acid methyl esters are analyzed by capillary column gas-chromatography (GC).

### Example 14

### Drug Screening Assay Using Yeast Microsomes

A. Yeast Microsome Preparation

A 2-5 1 culture of the OLE1 deletion mutant of Saccharomyces cerevisiae transformed with the 6×His tagged or non-tagged delta-9-desaturase is started with a cell density of approximately 3.2×10⁵ cells/ml (O.D.₆₀₀ 0.4) using the appropriate medium without galactose. After 8 h of incubation at 30° C. in an orbital shaker at 270 rpm, galactose is added to a final concentration of 2%. Yeast are further incubated for 12 h until they are harvested by centrifugation at 2060×g for 10 minutes at 4° C. and washed with water. The cell pellet is resuspended in ⅓ of its volume in a pH 7.2 isolation buffer (80 mM Hepes-KOH, 10 mM KCl, 320 mM sucrose, ²nM PMSF and a protease inhibitor cocktail). The cell suspension is poured into a mortar containing liquid N₂ and ground with sand using a ceramic pestle. The yeast powder is transferred to a conical test tube, to which ⅔ of the pellet volume of isolation buffer is added. The sand is removed by centrifugation at 57×g for 1 min and the suspension centrifuged at 10,000×g for 20 min to separate cell debris, nuclei and mitochondria. The supernatant is centrifuged at 106,000×g for 1 h to obtain the microsomal pellet, which is resuspended in 700 μl of isolation buffer. A protein assay is performed on the microsome suspension.

B. Incubation of Yeast Microsomes with Test Compounds

The activity of delta-9desaturase is determined by measuring the conversion of[1-¹⁴C]16:0 (palmitic acid) to [1-¹⁴C]16:1n-7 (palmitoleic acid). Reactions are started by adding 500 μg of yeast microsomal protein to pre-incubated tubes containing 0.20 μCi of the substrate fatty acid at a final concentration of 33 μM in 0.25 ml of 80 mM Hepes-KOH (pH 7.2) with 43.2 mM MgCl₂, 1.0 mM ATP, 500 μM NADH and 10 μM coenzyme A, and a range of concentrations of test compounds. The tubes are vortexed vigorously and after 15 min incubation in a shaking water bath (37° C.), the reactions are stopped by the addition of 2 ml of 10% (w/v) KOH in ethariol. Lipids in the incubation mixture are saponified at 80° C. for 45 min under N₂. The samples are then left in ice for 5 min before acidification. The fatty acids are extracted with hexane and esterified with BF₃ in methanol at 90° C. for 30 min. The fatty acid methyl esters are analyzed by HPLC as described in Example 13.

Results are expressed in pmol of palmitoleic acid produced/mg microsomal protein/min.

### Example 15

### Drug Screening Assay Using Purified Enzyme

A. Isolation of Delta-9-Desaturase from Yeast Microsomes

Yeast microsomes containing the delta-9-desaturase tagged with 6×His are stirred with Zwittergent 3-14 or mixtures of deoxycholate/Triton X100 (2% w/w) for 2 h at 4° C. to solubilize the delta-9-desaturase. Alternatively, yeast microsomes can be treated with 2.5% (v/v) water in acetone to improve the solubilizing power of the detergents. The mixture is centrifuged at 106,000×g for 1 h. The supernatant containing the enzyme is loaded onto a pre-equilibrated HiTrap chelating (Ni²⁺ charged iminodiacetate) column (Pharmacia) attached to a fast protein liquid chromatography system (Pharmacia). The column is washed with a 50 mM sodium phosphate buffer, pH 8.0. The tagged protein is eluted with the same buffer containing imidazole ranging from 0-500 mM and further concentrated by ultrafiltration using Centriprep (Amicon) concentrators.

B. Incubation of Delta-9-Desaturase with Test Compounds

The concentrated enzyme is incubated at 30-37° C. in Tris-HCl buffer (pH 7.2) containing 1 mM NADH, 80 μM cytochrome b₅, 4 μM NADH-cytochrome b₅ reductase, 6 mM egg phosphatidylcholine, 2% Triton X-100, 0.4% sodium deoxycholate, radiolabelled palmitoyl-CoA, and a range of concentrations of test compounds. After 15-90 min of incubation, the reaction is stopped and fatty acid methyl esters are analyzed as described in Example 13.

Alternatively, enzyme activity can be measured by the rate of NADH oxidation in the presence or absence of palmitoyl-CoA.

### Example 16

### Validation of Drug Screening Assays Described in Examples 13 to 15 Using Rat Liver Microsomes

A. Preparation of Rat Liver Microsomes

Wistar rats under light halothane (15% in mineral oil) anesthesia were sacrificed by exsanguination during periods of high enzyme activity. Livers are immediately rinsed with cold 0.9% NaCl solution, weighed and minced with scissors. All procedures are performed at 4° C. unless specified otherwise. Livers are homogenized in a solution (1:3 w/v) containing 0.25 M sucrose, 62 mM potassium phosphate buffer (pH 7.0), 0.15 M KCl, 1.5 mM N-acetylcysteine, 5 mM MgCl₂, and 0.1 mM EDTA using 4 strokes of a Potter-Elvehjem tissue homogenizer. The homogenate is centrifuged at 10,400×g for 20 min to eliminate mitochondria and cellular debris. The supernatant is filtered through a 3-layer cheesecloth and centrifuged at 105,000×g for 60 min. The microsomal pellet is gently resuspended in the same homogenization solution with a small glass/teflon homogenizer and stored at −70° C. The absence of mitochondrial contamination is enzymatically assessed. The protein concentration is measured using bovine serum albumin as the standard.

B. Incubation of Rat Liver Microsomes with Test Compounds

Reactions are started by adding 2 mg of microsomal protein to pre-incubated tubes containing 0.20 μCi of the substrate fatty acid (palmitic acid) at a final concentration of 33.3 μM in 1.5 ml of homogenization solution, containing 42 mM NaF, 0.33 mM niacinamide, 1.6 mM ATP, 1.0 mM NADH, 0.1 mM coenzyme A and a range of concentrations of test compounds. The tubes are vortexed vigorously and after 15 min incubation in a shaking water bath (37° C.), the reactions are stopped and fatty acids are analyzed as described in Example 13. Alternatively, fatty acid methyl esters are analyzed by capillary column gas chromatography (GC).

### Example 17

### Drug Screening Assay for Apolipoprotein A-1

A test compound that interacts with ApoA-1 is likely to interfere with the function of ApoA-1 as a cofactor leading to a loss in LCAT activity. A drug screening assay is based on well known enzymatic assays for LCAT activity (Li M. and Pritchard P. H., 2000, J. Biol. Chem. 275: 18079-18084 and Sorci-Thomas et al., 2000, J. Biol. Chem. 275: 12156-12163).

Briefly, purified recombinant ApoA-1 or purified plasma ApoA-1 is mixed with [³H]cholesterol and phosphatidylcholine at a molar ratio of 0.8:12.5:250 (˜1.2 μg) in 10 mM Tris-HCl pH 7.4, 140 mM NaCl, 0.25 mM EDTA, 0.15 mM sodium azide, 0.6% fatty acid-free bovine serum albumin and 2 mM beta-mercaptoethanol. A test compound is provided in the reaction at an appropriate concentration. Appropriate volume of fresh plasma is added to the reaction mix to provide LCAT. After a 30 min incubation at 37° C. the reaction is stopped by adding ethanol. The conversion of [³H]cholesterol to [³H]cholesterol ester is determined by extracting total lipids and separating them using silica gel G plates and hexane/diethyl ether/acetic acid (70:12:1 v/v/v) in thin layer chromatography techniques. The radioactivity of the cholesterol ester spot is measured by liquid scintillation counting, by scanning the plate with a Berthold radioisotope scanner or by autoradiographic procedures known in the art.

Variation(s) in the test reaction versus control reaction (without test compound) is a measure of the modulating effect of the test compound on ApoA-1.

Alternatively, small peptides or affibodies, obtained from combinatorial libraries adapted for phage display, that bind to ApoA-1 are identified by using a similar methodology described in Nord et al. 1997, Nat. Biotechnol., 15: 772-777.

### Example 18

### Drug Screening Assay for Glycerol-3-Phosphate Acyltransferase

Whole cells, spheroplasts, microsomes, mitochondria or the purified enzyme are obtained from acyltransferase mutants of Saccharomyces cerevisiae transformed with human or mammalian genes that encode 6×His tagged or non-tagged GPAT as described in Examples 13, 14 and 15.

Cells or organelles (i.e., microsomes or mitochondria) or the purified enzyme are incubated for 2-12 h (cells) or 5-20 min (organelles or purified enzyme) with radiolabelled glycerol and palmitoyl-CoA or other acyl-CoAs in the presence of different concentrations of the test compounds. The reaction is stopped with butanol and the lysophosphatidic acid formed separated by two dimensional thin layer chromatography using silica gel G plates and a mixture of solvents. The first dimension is run in chloroform:methanol:7 N ammonia (65:25:4 v/v/v) and the second dimension in chloroform:methanol:acetic acid:water (170:15:15:2 v/v/v/v). The silica with the radiolabelled lysophosphatidic acid spot is scraped and the radioactivity counted in a scintillation counter. Alternatively, HPLC methods with mass detector/on line ¹⁴C-detectors may be used.

### Example 19

### Drug Screening Assays for Spot-14

Spot-14 is a nuclear protein with all the characteristics of a transcription factor. It was shown by a yeast two-hybrid system that Spot-14 has a strong propensity for homodimerization, as is the case for many transcription factors (Cunningham et al., 1997, Endocrinology, 138: 5184-5188).

Therefore, a yeast two-hybrid system is used to screen compounds that disrupt the homodimerization of Spot-14. The two-hybrid assay is performed as described in Example 21. Spot-14 cDNA is cloned in a pray vector as well as in a bait vector containing DNA activation domain (AD) and DNA binding domain (DBD), respectively. The vectors are introduced into a suitably engineered yeast strain. Homodimerization (interaction of domains) of Spot-14 will bring the AD into close proximity to the DBD, thereby triggering the expression of a reporter gene. Expression of the reporter gene is assayed using techniques well known in the art. Treatment of the transformed yeast with any test compound that disrupts Spot-14 dimerization results in growth inhibition of the yeast on selective medium.

### Example 20

### Drug Screening Assay for METP

Several genes involved in mitochondrial function ate known in yeast (Belenkiy et al., 2000, Biochem. Biophys. Acta, 1467: 207-218 and Contamine V. and Pichard M., 2000, Microbiol. Mol. Biol. Rev., 64: 281-315). Yeast mutants that are defective in certain mitochondrial functions show several phenotypes including temperature sensitivity and growth inhibition on glycerol or ethanol. Such yeast mutants are transformed with the METP gene and phenotypic revertants analyzed. Complementation of yeast mutant phenotypes by related human genes is well known in the art (Nakashima et al., 1997, J. Biol. Chem., 272: 9567-9572 and Amaravadi et al., 1997, Hum. Genet., 99: 329-333). The revertant strains containing METP are used to screen for new chemical entities that inhibit the ability of METP to complement the mutant phenotype.

Two yeast genes, YMC1 and YMC2, encoding putative mitochondrial carrier proteins (Graf et al., 1993, Yeast, 9: 301-305) share about 30% identity with METP, indicating relatedness of function. The phenotypes of single YMC1 and YCM2 mutants or the double mutant are determined. The phenotype is complemented with METP, and then used for drug screening as described above.

### Example 21

### Drug Screening Assays Using Yeast 1- or 2-Hybrid Systems

The methods for the yeast one-hybrid and two-hybrid assays are known by persons skilled in the art (Fields S. and Song O., 1989, Nature, 340: 245-246; Ulmasov et al., 1997, Science, 276: 1865-1868 and Furuyama K. and Sassa S., 2000, J. Clin. Invest., 105, 757-764). Reagents or kits are commercially available for the assays, for example, the Hybrid Hunter Yeast Two-Hybrid and RNA-Protein Hybrid Hunter Systems (Invitrogen), the Matchmaker One-Hybrid and Two-Hybrid Systems (Clontech) and the HybriZAP Two Hybrid System (Stratagene).

The following assays are suitable for all of the genes regulated by fatty acids disclosed herein.

A. Yeast 1-Hybrid Assay

The known target elements, or control region ‘bait’ is inserted upstream of a reporter gene (e.g. HIS3) and integrated into the yeast genome to make a new reporter strain. The yeast strain is transformed with an activation domain (AD) fusion library to screen for DNA binding proteins that interact with the bait DNA sequence. Binding of an AD/DNA-binding domain (DBD) hybrid protein to the target sequence results in activation of the reporter gene transcription and subsequent selection. For example, expression HIS3 will allow colony growth on minimal medium lacking histidine. The cDNA encoding DNA binding protein (DBP) is isolated and characterized. The interaction is reconstructed in vitro or in vivo for screening test compounds by exposing the target elements or control region to the DBP in the presence of test compounds. The effect of the test compound is evaluated through assays, well known to those skilled in the art, that measure DNA/protein binding interactions.

B. Yeast 2-Hybrid Assay

Two physically distinct functional domains are necessary: a DNA binding domain (DBD) and an activation domain (AD). The polypeptide of interest is cloned into a “bait” vector, and expressed as a hybrid protein with a DBD. A library of cDNAs encoding potential interacting proteins is cloned in frame with AD in the “prey” vector. The bait and prey vector fusion constructs are transformed into one of several engineered yeast strains. If an interaction between bait and prey hybrid proteins occurs, the AD of the prey is brought into close contact with the DBD and transcription of the reporter gene is activated. Positive interacting proteins are easily identified by plating on nutrient deficient medium, and screening for reporter activity. The interaction between these two proteins is reconstructed in vitro or in vivo for screening test compounds by exposing the two interacting proteins in the presence of test compounds. The effect of the test compound is evaluated through assays, well known to those skilled in the art, that measure protein/protein binding interactions.

### Example 22

### Drug Screening Assays for INSIG1

A stable overexpressing INSIG1 cell line is established and a non-expressing cell line serves as a control. Cell membranes from the overexpressing and control cell lines are isolated and incubated with labeled test compounds. After an appropriate incubation time, the membranes are recovered and washed to remove unbound test compound. By techniques well known to those skilled in the art, the specific binding of the test compound is measured by comparing the amount bound to the membranes containing INSIG1 to that bound to control membranes.

Alternatively, the hepatocyte cell line, H35 is used with and without induction of INSIG1 by insulin. Alternatively, isolated membranes from regenerating liver cells after hepatectomy are used and compared to membranes from non-hepatectomized animals. In certain instances, whole cells rather than isolated membranes may be used. Alternatively, purified ISIG1 is used to generate an artificial recombinant INSIG1 /membrane complex and compared to membrane complexes without INSIG1.

### Example 23

### Drug Screening Assay for Glucose-6-Phosphate Dehydrogenase Using Yeast Complementation

G6PD null mutants of Saccharomyces cerevisiae do not grow in a medium lacking an organic sulfur source, i.e. 0.1 mM L-methionine, 0.5 mM L-cysteine, 0.2 mM glutathione, 0.2 mM homocysteine or 0.2 mM S-adenosylmethionine, or a mineral sulfur source, i.e. 0.5 mM sulfate, 0.5 mM sulfite or 0.5 mM sulfide (Thomas et al., 1991, EMBO J., 10: 547-553). Such yeast mutants are transformed with the G6PD gene and phenotypic revertants analyzed. Complementation of yeast mutant phenotypes by related human genes is well known in the art (Nakashima et al., 1997, J. Biol. Chem., 272: 9567-9572 and Amaravadi et al., 1997, Hum. Genet., 99: 329-333). The revertant strains containing G6PD are used to screen for new chemical entities that inhibit the ability of G6PD to complement the mutant phenotype.

This model, using whole or permeabilized yeast cells or spheroplasts is novel and useful to screen test compounds that affect the G6PD activity.

### Example 24

### Drug Screening Assay for Glucose-6-Phosphate Dehydrogenase Using Purified or Crude Enzyme Extract

Large volumes of the G6PD null mutant yeast transform ed with the G6PD gene are grown in appropriate minimal medium with the adequate selection system. The overproduced G6PD protein is isolated using affinity or anion exchange chromatography. A yeast homogenate is obtained using the liquid N₂ method as previously described (see Example 14) and applied to a 2′,5′-ADP-Sepharose column. The G6PD protein is eluted using a 2 buffer system. The first elution buffer is Tris-HCl, pH 7.6 containing 5 mM EDTA and 0.02% beta-mercaptoethanol, and the second elution buffer is 0.1 M potassium phosphate, pH 7.0. The protein concentration is measured by methods well known in the art. Alternatively, the crude yeast extract is used as a source of G6PD protein.

Modulation of G6PD activity by different test compounds is determined spectrophotometrically by following the increase in fluorescence at 341 nm due to the conversion of NADP⁺ to NADPH. The enzymatic reaction is performed in a cuvette containing 100 μl of the purified enzyme or the crude yeast extract, 900 μl of Tris-HCl 50 mM (pH 8.1), 1 mM MgCl₂, 200 μM glucose-6-phosphate, 100 μM NADP⁺ and a range, of concentrations for each test compound. The specific activity of the enzyme is calculated using a molar extinction coefficient of 6270 for NADPH.

### Example 25

### Drug Screening Assay for GLOL Using Yeast

Glyoxalase genes in Saccharomyces cerevisiae such as GLO2 and GLO4 have significant homology with mammalian GLOL. Yeast glo2/glo4 double deletion mutants (without glyoxalase II activity) are suitable hosts for transformation with the GLOL gene. Saccharomyces cerevisiae glo2/glo4 double deletion mutant transformed with GLOL gene is grown in YPD (2% glucose, 2% peptone, 1% yeast extract) or synthetic minimal medium (0.17% yeast nitrogen base, 0.5% ammonium sulfate) containing the appropriate amino acids and carbon source (2% glucose or 3% glycerol). Spheroplasts from the transformed yeast are obtained as described in Example 13. The whole yeast cells are permeabilized with alcohol, i.e. methanol, ethanol or isopropyl alcohol. Both cell models are incubated with permeable glutathione conjugates and 5,5′-dithiobis(2-nitrobenzoic acid). The hydrolysis of the thiol esters is followed at 412 nm using techniques known in the art to monitor GLOL activity. Test compounds are added at different concentrations and GLOL activity measured.

### Example 26

### Drug Screening Assay for GLOL Using Yeast Complementation

Yeast glo2/glo4 double deletion mutants do nt grow in the presence of methylglyoxal. Such yeast mutants are transformed with the GLOL gene and phenotypic revertants analyzed. Complementation of yeast mutant phenotypes by related human genes is well known in the art (Nakashima et al., 1997, J. Biol. Chem., 272: 9567-9572 and Amaravadi et al., 1997, Hum. Genet. 99: 329-333). The revertant strains containing GLOL are used to screen for new chemical entities that inhibit the ability of GLOL to complement the mutant phenotype.

This model, using whole or permeabilized yeast cells or spheroplasts is novel and useful to screen test compounds that affect the GLOL activity, thereby increasing or decreasing sensitivity to methylglyoxal.

### Example 27

### Drug Screening Assay for GLOL Using Purified Enzyme

Large volumes of the glo2/glo4 double mutant yeast transformed with the GLOL gene are grown and the overproduced GLOL protein is isolated by obtaining a yeast homogenate using the liquid N₂ method as described in Example 14. The homogenate is applied to a column with immobilized glutathione and the GLOL protein is eluted with 3 M NaCl in MOPS buffer (pH 7.2). The protein concentration is measured by methods well known in the art.

Modulation of GLOL activity is determined at 37° C. in 1 ml of 100 mM MOPS, pH 7.2 containing glutathione conjugates and 5,5′-dithiobis(2-nitrobenzoic acid). The reaction also contains GLOL at concentrations ranging from 7-200 ng/ml and appropriate concentrations of the test compound. The enzyme activity is measured by following the decrease of absorbance at 240 nm or by following the formation of glutathione by the increase of absorbance at 412 nm.

### Example 28

### Drug Screening Assay for Fatty Acid Synthase

Large volumes of the FAS null mutant yeast transformed with the FAS gene are grown in appropriate minimal medium with the adequate selection system. A yeast homogenate is obtained using the liquid N₂ method as previously described in Example 14. The overproduced FAS protein from the yeast homogenate is isolated using stepwise polyethylene glycol and ammonium sulfate precipitation, gel filtration, and anion exchange chromatography. The protein concentration is measured by methods well known in the art.

Alternatively, the crude yeast extract is used as a source of FAS protein.

Modulation of FAS activity by different test compounds is determined spectrophotometrically by following the oxidation of NADPH to NADP⁺at 340nm or using HPLC techniques in which the incorporation of radiolabelled acetyl-CoA or malonyl-CoA into palmitic acid is analyzed.

### Example 29

### Drug Screening Assay for Delta-3, delta-2-enoyl-CoA Isomerase Using Yeast Complementation

Complementation of yeast mutant phenotypes by related human or mammalian genes is well known to those skilled in the art (Nakashima et al., 1997, J. Biol. Chem., 272: 9567-9572 and Amaravadi et al., 1997, Hum. Genet., 99: 329-333). The Saccharomyces cerevisiae delta-3, delta-2-enoyl-CoA isomerase mutant does not grow in a test medium containing oleic acid (0.1% yeast extract, 0.67% yeast nitrogen base, 0.02% Tween40, 0.1% oleic acid and 0.1% dextrose; or 1% yeast extract, 2% bacto-peptone, 0.2% oleic acid, and 0.02% Tween40). Such a yeast mutant is transformed with a. plasmid containing the mammalian delta-3, delta2-enoyl-CoA isomerase gene using techniques well known in the art, and phenotypic revertants analyzed in test medium. The revertant strains containing delta-3, delta-2-enoyl-CoA isomerase are used to screen for new chemical entities that inhibit the ability of delta-3,-delta-2-enoyl-CoA isomerase to complement the mutant phenotype. This model, using whole or permeabilized yeast cells or yeast spheroplasts is appropriate to screen test compounds that affect the delta-3, delta-2-enoyl-CoA isomerase function, thereby increasing or decreasing requirement for oleic acid.

### Example 30

### Drug Screening Assay for Delta-3, delta-2-enoyl-CoA Isomerase Using Purified Enzyme

Large volumes of the delta-3, delta-2-enoyl-CoA isomerase mutant yeast transformed with mammalian delta-3, delta-2-enoyl-CoA isomerase gene are grown, and the overproduced protein is obtained from yeast homogenate using the liquid N₂ method as described in Example 14. The mitochondrial pellet is isolated from the homogenate using differential centrifugation techniques. The pellet is resuspended in 100 ml of 25 mM phosphate buffer pH 7.4, and sonicated for 2 minutes in an ice bath. The suspension is heated at 70° C. for 30 seconds, cooled on ice and centrifuged at 15,000×g for 15 minutes at 4° C. The mitochondrial lysate is dialyzed against 25 mM phosphate buffer (pH 6.0), and loaded onto a CM52 cellulose column with bed volume of 70 ml. Unbound protein is eluted with 25 mM phosphate buffer pH 6.0, and the delta-3, delta-2-enoyl-CoA isomerase eluted with 30 ml of 25 mM phosphate buffer pH 7.4. The eluate is dialyzed against 20 mM Tris-HCl, pH 8.7 and separated or a fast protein liquid chromatography mono-Q anion exchange column by gradient elution with 20 mM Tris-HCl pH 8.7 and 20 mM Tris-HCl pH 8.7 containing 1 M NaCl. The delta-3, delta-2-enoyl-CoA isomerase elutes at 50 mM NaCl. Protein concentration is measured by methods well known in the art.

Modulation of delta-3, delta-2-enoyl-CoA isomerase activity by test compounds is determined spectrophotometrically at 340 nm according to Binstock and Schulz (1981, Meth. Enzymol., 71: 403-411). The reaction contains appropriate concentration of substrate (3-cis-octenoyl-CoA or 3-trans-hexenoyl CoA), test components and purified enzyme.

Alternatively, whole yeast, permeabilized yeast or spheroplasts, containing the recombinant delta-3, delta-2-enoyl-CoA isomerase, are used for the assay. The yeast used in these assays can also be genetically engineered to produce endogenous specific substrates for the delta-3, delta-2-enoyl-CoA isomerase.

### Example 31

### Drug Screening Assay for COMT Using Crude Lysate or Purified Enzyme

To produce sufficient amounts of active recombinant mammalian COMT in E. coli or a suitable host system, the gene is introduced into a bacterial expression vector, for example pKEX 14. COMT is produced at high levels (up to 10% of total bacterial protein) after induction of the T7 RNA polymerase gene with IPTG. The enzyme is purified from E.coli cells using procedures known in the art (Lundstrom et al., 1992, Biochim. Biophys. Acta, 1129: 149-154). The purified enzyme is used for screening drugs that modulate COMT activity. Alternatively, the crude bacterial lysate is used for the assay.

Modulation of COMT activity by test compound is determined using methods known in the art (Tilgmann C. and Kalkkinen N., 1990, FEBS Lett., 264: 95-99 and Lautala et al., 1999, J. Chromatogr. B, 736: 143-151). Briefly, 250 μl of the incubation mixture containing 5 mM MgCl₂, 20 mM L-cysteine, 0.15 mM [¹⁴C]S-adenosyl-L-methionine (0.1 μCi), and 1-100 μg of mammalian COMT (provided as purified enzyme or crude lysate) in appropriate buffer is pre-incubed at 37+ C. for 5 min. Subsequently, 0.5 mM catechol (as substrate) and appropriate of test compounds are added to the incubation mixture. After a 10 to 30 min incubation period, the reaction is terminated by the addition of 25 μl cold 4 M perchloric acid. The sample is kept on ice for 10 min and subsequently centrifuged for 5 min at 22,000×g.

The methylated catechol products are separated from S-adenosyl-L-methionine using a Hewlett-Packard HPLC chromatograph (1090 Series II) equipped with an inline radioisotope flow detector with a Beckman 110B cocktail pump using a 1000 μl liquid scintillation flowcell with an exit volume of 20 μl. Alternatively, a 300 μl flowcell packed with silanised cerium activated platinum glass as scintillant is used.

Alternatively, whole yeast or permeabilized yeast or spheroplasts, containing the recombinant COMT, is used for the drug screening assay.

### Example 32

### Drug Screening Assay Using Human SCD Control Region

Plasmid pGh3022.1, containing the human SCD control region, is used to screen drugs that modulates the human SCD promoter activity. Transient transfections are performed to evaluate the functionality of the SCD control region using techniques known by persons skilled in the art.

HepG2 cells are transfected with 10 μg of pGh3022.1 (Example 7) and 1 μg of vector pRSV-NEO (ATCC), using 10 μl of Lipofectamine 2000 Reagent (Gibco BRL) in a 60 mm tissue culture dish as described by the manufacturer. After a 24 h incubation, the cells are passaged into two 150 mm tissue culture dishes at a 1:2 dilution and grown for another 24 h. Geneticin (Gibco BRL) is added to the medium at a concentration of 800 μg/ml. After 3-4 weeks of growth under the selection pressure of the antibiotic, the resistant clones are isolated and characterized for their luciferase activity.

Drug screening is performed using the Luciferase Enzyme Assay System (Promega), following the manufacturer's recommendations. Briefly, transfected cells grown in a 96 well plate are exposed to test compound. After an appropriate incubation time, the cells are washed with Mg²⁺ and Ca²⁺ free PBS Cells are lysed with 20 μl of 1× Luciferase Cell Culture Lysis Reagent (CCLR, Promega). The plate is placed onto luminometer with automatic injector. The injector adds 100 μl of Luciferase Assay Reagent (Promega) per well, and the light emission generated by the reaction is read for 10 seconds after a 2 second delay before moving to the next well to repeat the process. Cell cultures without a test compound are used as controls. Any significant difference in the luciferase activity indicates that the test compound is modulating the human SCD promoter activity.

This assay or other reporter assay is useful for drug screening using the control region of any fat regulated gene.

### Example 33

### Drug Screening Assay Using Antibodies Against Polypeptides Encoded by Fat Regulated Genes in Animal Models

Monoclonal antibodies against the polypeptides encoded by fat regulated genes are prepared using methods well known in the art.

Rats are randomly divided into test and control groups. A test compound and placebo are enterally or parenterally administered to the test and control groups, respectively. The test compound and placebo are provided by bolus or continuous administration. After an appropriate period of treatment, animals are sacrificed by exsanguination, and tissue samples taken for immunoassays.

In blood, the amount of excreted target polypeptide that is encoded by the fat regulated gene, is determined by centrifuging the blood and exposing the plasma to the corresponding monoclonal antibody. The amount of antigen-antibody complex is detected using a secondary antibody which is linked to a marker. These immunological products are quantified using methods well known in the art (e.g., immunofluorescence or chemiluminiscence).

Similarly, whole organs or leukocytes from blood of test and control animals are homogenized as described in Example 16. Cell debris is removed by centrifugation and the supernatant is used for the immunoassays as described herein.

The assay is also suitable for the identification of drugs that alter the expression of fat regulated genes in normal or disease (e.g., diabetes or caner) animal models.

### Example 34

### Drug Screening Assay Using Antibodies Against Polypeptides Encoded by Fat Regulated Genes in Cell Culture

HepG2 cells are grown in a medium with (test group) or without (control group) a test compound. After an appropriate period of treatment, the medium is collected, cells are washed with PBS and lysed using techniques known in the art. The medium containing the excreted target polypeptide that is encoded by the fat regulated gene, and the cell lysate are used for the immunoassay described in Example 33.

### Example 35

### Drug Screening Assay Using Affinity Chromatography and Antibodies Against Polypeptides Encoded by Fat Regulated Genes

A monoclonal antibody described herein (Example 33) is immobilized to a protein G POROS column (PerSeptive Biosystems, Mass.) using a crosslinking agent known by persons skilled in the art (Evans et al., 1996., Nat. Biotech. 14: 504-507). A column without the monoclonal antibody and treated with crosslinking reagents is used as control.

A natural or synthetic peptide combinatorial library is injected onto the column. After washing the unbound material with several column volumes of PBS pH 7.4, the bound peptide is eluted with 12 mM HCl and characterized using methods known to persons skilled in the art (e.g., reverse phase HPLC developed with a gradient of acetonitrile and mass spectroscopy).

This assay is used for the identification of antigenic test compounds with high affinity to monoclonal antibodies raised against polypeptides encoded by fat regulated genes. The identified test compound, due to its similar antigenic properties to the target polypeptide, is used to mimic the function of the said target polypeptide in disease.

### Example 36

### Diagnostic Assay Using Antibodies

Fractionated blood (e.g. plasma and leukocytes) obtained from subjects with suspected alterations in the production of the target polypeptide is used for the diagnosis of the associated disease. Leukocytes are lysed using techniques known to those skilled in the art.

The amount of target polypeptide that is encoded by the fat regulated gene, is determined by exposing the plasma and the leukocyte lysate to the corresponding monoclonal antibody. The amount of antigen-antibody complex is detected using a secondary antibody which is linked to a marker. These immunological products are quantified using methods well known in the art (e.g., immunofluorescence or chemiluminiscence).

### Example 37

### Determination of Tissue Distribution of RNA by Northern Blot Analysis

A. Rat

Wistar rats (˜300 g) were obtained from Charles River Canada Ltd. and maintained on regular chow for 5-6 days. Each rat was sacrificed via cardiac puncture under anesthesia and its tissues immediately perfused with saline. Appropriate tissues were quickly removed and frozen in liquid nitrogen.

RNA was isolated from liver, brain, kidney, lung, ovary, spleen, heart, skeletal muscle, abdominal adipose tissue and nerve of female rats using Trizol reagent (Gibco BRL) as per the manufacturer's protocol. The RNA isolated from spleen, heart, muscle, adipose tissue and nerve was subsequently purified using RNeasy (Qiagen) as per the manufacturer's protocol. Additionally, RNA was isolated from testes of male rats as described in Auffray C. and Rougeon F. (1980, Eur. J. Biochem., 107: 303-314). The testes were ground to a fine powder under liquid nitrogen and homogenized in 3 M LiCl and 6 M urea. The RNA was precipitated at 4° C. and pelleted by centrifugation. The pellet was resuspended and extracted with chloroform/isoamyl alcohol prior to ethanol precipitation.

For each tissue, 10 μg of total RNA pooled from 6 rats (for nerve) or 4 rats (for all tissues except nerve), was subjected to formaldehyde agarose gel electorphoresis and transferred to a nylon membrane (BioRad) by capillary action using standard procedures (Sambrook et al., 1989, Molecular Cloning, 2nd Edition, Cold Spring Harbor Laboratory Press, Cold Spring Harbour, N.Y.).

Northern blot analysis was carried out using standard procedures (Ausubel et al., 1994-, Current Protocols in Molecular Biology, John Wiley & Sons, NY). Probes were prepared by labelling cDNA using [alpha-³²P]dCTP and Rediprime II Random Prime Labelling System (Amersham Pharmacia Biotech). The cDNA probes for rat METP, FTF1, A1BG and GLOL obtained during the differential display study (Example 1) were used. The membrane was washed at high stringency using 0.25× SSC, 0.1% SDS at 55° C. The membrane was probed with a nucleotide probe against the 18S rRNA subunit for normalization of loading.

B. Human

A membrane containing poly(A)⁺ RNA from 12 different human tissues (brain, heart, skeletal muscle, colon, thymus, spleen, kidney, liver, small intestine, placenta, lung and peripheral blood leukocytes) was purchased from Clontech (Human 12-lane MTN blot). Northern blot analysis was carried out using standard procedures (Ausubel et al., 1994-, Current Protocols in Molecular Biology, John Wiley & Sons, NY). Probes were prepared by labelling cDNA using [alpha-³²P]dCTP and Rediprime II Random Prime Labelling System (Amersham Pharmacia Biotech). The cDNA probe for METP corresponded to the complete coding sequence for the human gene. The cDNA probes for GLOL, FTF1 and A1BG corresponded to bases 1-652, 564-1363 and 214-915, respectively, of the coding sequences of the human genes. The membrane was washed at high stringency using 0.25×SSC, 0. 1% SDS at 55° C.

References

U.S. Pat. No. 3,817,837, June, 1974, Rubinstein et al.

U.S. Pat. No. 3,850,752, November, 1974, Schuurs et al.

U.S. Pat. No. 3,939,350, February, 1976, Kronick et al.

U.S. Pat. No. 3,996,345, December, 1976, Ullman et al.

U.S. Pat. No. 4,275,149, June, 1981, Litman et al.

U.S. Pat. No. 4,277,437, July, 1981, Maggio

U.S. Pat. No. 4,366,241, December, 1982, Tom et al.

U.S. Pat. No. 4,399,216, August, 1983, Axel et al.

U.S. Pat. No. 4,704,362, November. 1987Itakura et al.

U.S. Pat: No: 4,766,075, August, 1988, Goeddal et al.

U.S. Pat. No. 4,784,950, November, 1988, Hagen et al.

U.S. Pat. No. 4,801,542, January, 1989, Murray et al.

U.S. Pat. No. 4,816,567, March, 1989, Cabilly et al.

U.S. Pat. No. 4,935,349, June, 1990, McKnight et al.

U.S. Pat. No. 5,130,238, July, 1992, Malek

International Patent Application No. WO 88/04300, June, 1988, Cech et al.

Patent Cooperation Treaty International Publication No. WO 93/05182, Mar., 1993, Bruice

European Published Application No. 0320308, June,1989, Backman et al.

International Patent Application No. WO 00/51430, September, 2000, Bergsma et al.

Adediran,1996, Biochimie, 78: 165-170

Altschul et al., 1990, J. Molec. Biol., 215: 403-410

Amaravadi et al., 1997, Hum. Genet., 99: 329-333

Armstrong et al., 1991, Biochem. Biophys. Res. Commun., 177: 1056-1061

Askar et al., 1996, Indian J. Biochem. Biophys., 33: 512-518

Auffray C. and Rougeon F., 1980, Eur. J. Biochem., 107: 303-314

Ausubel et al., 1994-, Current Protocols in Molecular Biology, John Wily & Sons, Inc., NY

Bautista et al., 1992, Biochim. Biophys. Acta, 1119: 74-80

Been M. D. and Cech T. R., 1986, Cell, 47: 207-216

Belenkiy et al., 2000, BBA, 1467: 207-218

Bell R. M., Coleman R. A., 1980, Annu. Rev. Biochem., 49: 459-487

Bennett et al., 1995, J. Mol. Recognit., 8: 52-58

Berthou et al., 1995, Eur. J. Biochem. 232: 179-187

Binstock J. F. and Schulz H., 1981, Meth. Enzymol., 71: 403-411

Borges et al., 1998, J. Enzyme Inhib., 13: 473-483

Bortoff et al., 1997, Endocrine, 7: 199-207

Breslow et al., 1982, Proc. Natl. Acad. Sci., 79: 6861-6865

Cameron et al., 1993, Br. J. Pharmacol., 109: 972-979

Cameron et al., 1999, Structure Fold. Des. 7: 1067-1078

Carillo H. and Lipman D., 1988, SIAM J. Applied Math., 48:1073

Caskey C. T., 1987, Science, 236:1223-1229

Catanese J. J. and Kress L. F., 1992, Biochemistry, 31: 410-418

Chin et al., 1995, Am. J. Physiol., 269; :E691-E700

Chong B. S. & Mersfelder T. L, 2000, Ann Pharmacother., 34: 1056-1065

Church et al., 1988, Proc. Natl. Acad. Sci., 81: 1991-1995

Clarke et al., 1990, J. Nutr., 120: 218-224

Clarke et al., 1990, J. Nutr., 120: 225-231

Connolly B. A., 1987, Nucl. Acid Res., 15: 3131-3139

Contamine V. and Pichard M., 2000, Microbiol. Mol. Biol. Rev., 64: 281-315

Copsey et al., 1988, Genetically Engineered Human Therapeutic Drugs, Stockton Press, New York

Cotter et al., 1995, DiabeticNeuropathy: New Concepts and Insights,Elsevier Science B. V., Amsterdam, pp. 115-120

Cotton et al., 1985, Proc. Natl. Acad. Sci., 85: 4397-4401

Cserzo et al., 1997, Prot. Eng., 10: 673-676

Cunningham et al., 1997, Endocrinology, 138: 5184-5188

Cunningham et al., 1998, Thyroid, 8: 815-825

Dao et al., 1982, Proc. Natl. Acad. Sci., 79: 2860-2864

Davis et al., 1986, Basic Methods in Molecular Biology, Elsevier, NY, N.Y.

Deutcher, M., ed., 1990, Guide to Protein Purification. Meth. in Enzymol., 182

Devereux et al., 1984, Nucl. Acid Res., 12(1): 387-395

Diamond et al., 1993, J. Biol Chem., 268: 15185-15192

Dines et al., 1993, Diabetologia, 36:1132-1138

Dines et al., 1996, Prostaglandins, Leukotrienes and Essential Fatty Acids, 55: 159-165

Erickson et al., 1992, Ann. Rep. Med. Chem., 27: 271-289

Ericsson et al., 1997, J. Biol. Chem., 272: 7298-7305

Evans et al., 1996., Nat. Biotech. 14: 504-507

Fields S. and Song O., 1989, Nature, 340: 245-246

Flavell et al., 1978, Cell,15: 2541

Foretz et al., 1999, Biochem. J., 34: 371-376

Furuyama K. and Sassa S., 2000, J. Clin. Invest., 105: 757-764

Ganesh et al., 1999, Bioche. Biophys. Acta, 1439: 415-423

Geever et al., 1981, Proc. Natl. Acad. Sci., 78: 5081-5085

Goding J. W., 1996, Monoclonal Antibodies: Principles and Practice: Production and Application of Monoclonal Antibodies in Cell Biology, Biochemistry and Immunology, 3^{rd} edition, Academic Press, NY

Graf et al., 1993, Yeast, 9: 301-305

Gribskov M. and Devereux J., eds., 1991, Sequence Analysis Primer, M Stockton Press, NY

Griffin A. M. and Griffin H. G., eds., 1994, Computer Analysis of Sequence Data, Part 1, Humana Press, NJ

Grillasca et al., 1997, FEBS Lett., 401: 38-42

Guldberg and Marsden, 1975, Pharmacol. Rev., 27: 135-306

Gurvitz et al., 1999, Biochem. J., 344: 903-914

Gyuris et al., 1993, Cell, 75: 791-803

Haber et al., 1993, Hepatology, 22: 906-914

Haddad et al., 1986, J. Biol. Chem., 261:13268-13277

Hamy et al., 1997, Proc. Natl. Acad. Sci., 94: 3548-3553

Hanahan et al., 1983, J. Mol. Biol., 166: 557-580

Harlow E. and Lane D.(eds.), 1988, Antibodies:A Laboratory Manual, Cold Spring Harbor, N.Y.

Haseloff J. and Gerlach W. L., 1988, Nature, 334: 585-591

Hassan et al., 2000, Proc. Natl. Acad. Sci., 97: 8883-8888

Henrik et al., 1997, Prot. Eng., 10: 1-6

Hinnen et al., 1978, Proc. Natl. Acad. Sci., 75: 1929-1933

Honma et al., 1999, Molecular Brain Research, 69: 93-103

Horrobin D. F., 1990, Pathophysiology and Roles in Clinical Medicine, Wiley-Liss; NY

Houbenweyl et al., 1987, Methods of Organic Chemistry, Wansch E. (ed), Vol. 15 I and II, Thieme, Germany

Hsu et al., 1996, J. Biol. Clem., 271: 13584-13592

Huse et al., 1989, Science, 246: 1275-1281

Hwang et al., 1999, Proc. Natl. Acad. Sci., 96: 12997-13002

Innis M. A. and Gelfand D. H., 1989, PCR Protocols, A Guide to Methods and Applications, Innis M A., Gelfand D. H., Shinsky J. J. and White T. J. (eds), Academic Press, NY, pp. 3-12

Ishioka et al., 1986, Proc. Natl. Acad Sci., 83: 2363-2367

Izant J. G. and Weintraub H., 1984, Cell, 36:1007-1015

Janssen et al., 1994, Genomics, 23: 223-228

Jayakumar et al., 1995, Proc. Natl. Acad. Sci., 92: 8695-8699

Jayakumar et al., 1996, Proc. Natl. Acad. Sci., 93: 14509-14514

Jerkins et,al., 1995, J. Biol. Chem., 270: 1416-1421

Johanson et al., 1995, J. Biol. Chem., 270: 9459-9471

Jorga et al., 2000, Clin Pharmacol Ther., 67: 610-620

Joyeux et al., 1990, J. Clin. Endocrinol Metab., 70: 1438-1444

Jump et al., 1990, Mol. Endocrinol., 4: 1655-1660

Jump et al., 1994, J. Lipid Res., 35: 1076-1084

Karlin et al., 1976, J. Lipid Res., 17: 30-37

Kastner et al., 1994, Neuroscience, 62: 449-457

Kim et al., 2000, J. Lipid. Res., 41: 1310-1316

Kinlaw et al., 1989, J. Biol. Chem., 264: 19779-19783

Kinlaw et al., 1993, Endocrinology, 133: 645-650

Kletzien et al., 1994, FASEB J., 8: 174-181

Kohler G. and Milstein C., 1975, Nature, 256: 495-497

Kraemer et al., 1993, J. Lipid Res., 34: 663-671

Kuhajda et al., 1994, Proc. Natl. Acad. Sci., 91: 6379-6389

Landegren et al., 1988, Science, 241: 1077-1080

Landegren et al., 1989, Science, 242: 229-237

Lautala et al., 1999, J. Chromatogr. B, 736: 143-151

Lesk A. M., ed., 1988, Computational Molecular Biology, Oxford University Press, NY

Li et al., 1994, Int. J. Cancer, 57: 348-352

Li M. and Pritchard P. H., 2000, J. Biol. Chem., 275: 18079-18084

Liang P. and Pardee A. B., 1992, Science, 257: 967-971

Liaw C. W. and Towle H. C., 1984, J. Biol. Chem., 259: 7253-7260

Liu H. C. and Towle H. C., 1994, Mol. Endocrinol., 8: 1021-1037

Llewellyn et al., 1987, J. Mol. Biol., 195: 115-123

Lundstrom et al., 1991, DNA Cell Biol., 10: 181-189

Lundstrom et al., 1992, Biochim. Biophys. Acta, 1129: 149-154

Lupas et al., 1991, Science, 252: 1162-1164

Mack E. W., 1990, Remington's Pharmaceutical Sciences, Mack Publishing Company, Easton, Pa., 13^{th} edition

Madsen et al., 1998, J. Lipid Research, 39: 583-593

Margolskee et al., 1988, Mol. Cell. Biol., 8: 2837-2847

Mazza G. and Domah B. D.(eds.), 2000, Herbs, Botanicals and Teas, Technomic Publishers, Lancaster, Pa.

McLaughlin et al.,1988, J. Virol., 62: 1963-1973

Mei et al., 1998, Biochemistry, 37: 14204-14212

Merrifield, 1964, J. Am. Chem. Assoc., 85: 2149-2154

Miyazaki et al., 2000, J. Biol. Chem., 275: 30132-30138

Moncur et al., 1998, Proc. Natl. Acad. Sci., 95: 6989-6994

Moore et al., 1986, Carcinogenesis, 7: 1419-1424

Moss et al., 1987, Annu. Rev. Immunol., 5: 305-324

Muller-Newten G. and Stoffel W., 1991, Biol. Chem. Hoppe-Seyler, 372: 613-624

Myers et al., 1985, Science, 230: 1242-1246

Myers et al., 1986, Cold Spring Harbour Sym. Quant. Biol., 51: 275-284

Nagase et al., 2000, DNA Res., 7: 273-281

Nakashima et al., 1997, J. Biol. Chem., 272: 9567-9572

Nord et al., 1997, Nat. Biotechnol., 15: 772-777

Ntambi et al., 1999, J. Lipid Res., 40: 1549-1558

Okano et al., 1988, EMBO J., 7: 3407-3412

Orkin et al.,1988, Prog. Med. Genet., 7: 130-142

Palosaari P. M. and Hiltunen J. K., 1990, J. Biol Chem., 265: 2446-2449

Peng et al., 1997, Genomics, 43: 278-284

Phillippe et al., 1994, Eur. J. Biochem., 226: 377-384

Qi et al., 1994, Toxicon, 32: 1459-1469

Qi et al., 1995, Toxicon, 33: 241-245

Rasmussen et al., 1987, Meth. Enzymol., 139: 642-654

Roe et al., 1990, J. Clin. Invest., 85: 1703-1707

Rosenberg et al., 1985, Nature, 313: 703-706

Saggerson E. D. and Carpenter C. A., 1987, Biochem. J., 243: 289-292

Saiki et al., 1985, Science, 230:1350-1353

Saiki, et al., 1986, Nature, 324:163-166

Salminen et al., 1990, Gene, 93: 241-247

Sambrook et al., 1989, Molecular Cloning, 2nd Edition, Cold Spring Harbor Laboratory Press, Cold Spring Harbour, N.Y.

Semenkovich C. F., 1997, Prog. Lipid Res., 36: 43-53

Shin et al., 1991, J. Biol. Chem., 266: 23834-23839

Smith D. W., ed., 1993, Biocomputing: Informatics and Genome Project, Academic Press, NY

Sorci-Thomas et al., 2000, J. Biol. Chem. 275: 12156-12163

Soutar et al., 1975, Biochemistry, 14: 3057-3064

Srivastava et al., 2000, Eur. J. Biochem., 267: 4272-4280 .

Taviaux et al., 1997, Cytogenet. Cell Genet., 76: 219-220

Tenhunen et al., 1994, Eur. J. Biochem., 223: 1049-1059

Tenhunen J., 1996, DNA Cell Biol., 15: 461-473

Thiede et al., 1986, J. Biol. Chem., 261: 1323-1325

Thomas et al., 1991, EMBO J., 10: 547-553

Tilgmann C. and Kalkkinen N., 1990, FEBS Lett., 264: 95-99

Tilgmann C. and Ulmanen I., 1996, J. Chromatogr. B Biomed. Appl., 684: 147-461

Tomlinson et al., 1998, J. Nutr., 118: 408-415

Ulmasov et al., 1997, Science, 276: 1865-1868

Ursini et al., 1990, Biochem. Biophys. Res. Commun., 170: 1203-1209

Vieira-Coelho M. and Soares-da-Silva P., 1999, Brain Res., 821: 69-78

von Heijne G., 1987, Sequence Analysis in Molecular Biology, Academic Press, NY

Wakil, 1989, Biochemistry, 28: 4523-4530

Waldmann T. A., 1991, Science, 252: 1657-1661

Wallace et al., 1986, Cold Spring Harbour Symp. Quant. Biol., 51: 257-261

Wanders et al., 1992, J. Inherit. Metab. Dis., 15: 643-644

Waters K. M. and Ntambi J. M., 1994, J. Biol. Clem., 269: 27773-27777

Wei et al., 2000, Ann. Neurol., 47: 286-296

Wilentz et al., 2000, Pediatr. Dev. Pathol., 3: 525-531

Xie et al., 1999, Mol. Pharmacol., 56: 31-38

Yet et al., 1993, Biochemistry, 32: 9486-9491

Yoshida et al., 1999, J. Nutr. Sci. Vitaminol., 45: 411-421

Zaug A. J. and Cech T. R, 1986, Science, 231: 470-475

Zaug et al., 1984, Science, 224: 574-578

Zaug et al., 1966, Nature, 324: 429-433

Zhang et al., 1999, Biochem. J., 340: 252-264

We claim:

1. An isolated polynucleotide segment, comprising a polynucleotide sequence which is selected from the group consisting of:
(a) a sequence comprising SEQ ID NO:1;
(b) a sequence comprising SEQ ID NO:3;
(c) a sequence comprising SEQ ID NO:6;
(d) a sequence comprising SEQ ID NO:11;
(e) a sequence comprising SEQ ID NO:13;
(f) a sequence comprising SEQ ID NO:16;
(g) a sequence comprising SEQ ID NO:18;
(h) a sequence comprising SEQ ID NO: 24;
(i) a sequence which is at least 80% homologous with a sequence of any of (a) to (h);
(j) a sequence which is at least 90% homologous with a sequence of any of (a) to (h);
(k) a sequence which is at least 95% homologous with a sequence of any of (a) to (h);
(l) a sequence which is at least 98% homologous with a sequence of any of (a) to (h);
(m) a sequence which is at least 99% homologous with a sequence of any of (a) to (h);
(n) a sequence which hybridizes to any of (a) to (m) under stringent conditions; and,
(o) a sequence with is a functional derivative of any of (a) to (m).

2. An isolated polynucleotide segment of claim 1, wherein the isolated polynucleotide segment is cDNA.

3. A vector comprising a polynucleotide segment of claim 1 in a suitable vector.

4. A host cell comprising a polynucleotide segment of claim 1 in a host cell which is heterologous to said segment.

5. An isolated polynucleotide fragment selected from the group consisting of:
(a) a sequence having at least 15 sequential bases of nucleotides of a segment of claim 1;
(b) a sequence having at least 30 sequential bases of nucleotides of a segment of claim 1; and,
(c) a sequence having at least 50 sequential bases of nucleotides of a segment of claim 1.

6. A vector comprising a polynucleotide segment of claim 5 contained in a vector which is heterologous to said segment.

7. A vector of claims 3 or 6, wherein said vector contains or encodes a tag.

8. An isolated polynucleotide segment, comprising a polynucleotide sequence which retains substantially the same biological function or activity as the polynucleotide encoded by a segment of claim 1.

9. A method for identifying a compound which inhibits or promotes the activity of a polynucleotide segment of claim 1, comprising the steps of:
(a) selecting a control animal having said segment and a test animal having said segment;
(b) treating said test animal using a compound; and,
(c) determining the relative quantity of an expression product of said segment, as between said control animal and said test animal.

10. A method of claim 9, wherein said animals are mammals.

11. A method of claim 10, wherein said mammals are rats.

12. A method for identifying a compound which inhibits or promotes the activity of a polynucleotide segment of claim 1, comprising the steps of:
(a) selecting a host cell of claim 4;
(b) cloning said host cell and separating said clones into a test group and a control group;
(c) treating said test group using a compound; and
(d) determining the relative quantity of an expression product of said segment as between said test group and said control group.

13. A method for identifying a compound which inhibits or promotes the activity of a polynucleotide segment of claim 1, comprising the steps of:
(a) selecting a test group having a host cell of claim 4, a part thereof or an isolated polynucleotide thereof, and a control group;
(b) treating said test group using a compound; and
(c) determining the relative quantity or relative activity of a product of said segment or of the said segment, as between said test group and said control group.

14. An isolated polypeptide segment comprising an isolated polypeptide selected from the group consisting of:
(a) a sequence comprising SEQ ID NO:2;
(b) a sequence comprising SEQ ID NO:4;
(c) a sequence comprising SEQ ID NO:7;
(d) a sequence comprising SEQ ID NO:12;
(e) a sequence comprising SEQ ID NO 14;
(f) a sequence comprising SEQ ID NO:17;
(g) a sequence comprising SEQ ID NO: 25;
(h) a sequence which is at least 80% homologous with a sequence of any of (a) to (g);
(i) a sequence which is at least 90% homologous with a sequence of any of (a) to (g);
(j) a sequence which is at least 95% homologous with a sequence of any of (a) to (g);
(k) a sequence which is at least 98% homologous with a sequence of any of (a) to (g); and,
(l) a sequence which is at least 99% homologous with a sequence of any of (a) to (g).

15. A host cell comprising a polypeptide segment of claim 14 in a host cell which is heterologo to said segment.

16. A process for producing a polypeptide of a segment of claim 14 comprising the step of culturing the host cell of claim 15 under conditions sufficient for the production of said polypeptide.

17. An isolated polypeptide segment, comprising a polypeptide sequence which retains substantially the same biological function or activity as the polypeptide in a segment of claim 14.

18. A method for identifying a compound which inhibits or promotes the activity of a polypeptide segment of claim 14, comprising the steps of:
(a) selecting a control animal having said segment and a test animal having said segment;
(b) treating said test animal using a compound;
(c) determining the relative quantity or relative activity of an expression product of said segment or of the said segment, as between said control animal and said test animal.

19. A method of claim 18, wherein said animals are mammals.

20. A method of claim 19, wherein said mammals are rats.

21. A method for identifying a compound which inhibits or promotes the activity of a polypeptide segment of claim 14, comprising the steps of:
(a) selecting a host cell of claim 15;
(b) cloning said host cell and separating said clones into a test group and a control group;
(c) treating said test group using a compound; and
(d) determining the relative quantity or relative activity of an expression product of said segment or of the said segment, as between said test group and said control group.

22. A method for identifying a compound which inhibits or promotes the activity of a polypeptide segment of claim 14, comprising the steps of.
(a) selecting a test group having a host cell of claim 15, a part thereof or an isolated polypeptide thereof and a control group;
(b) treating said test group using a compound; and
(c) determining the relative quantity or relative activity of a product of said segment or of the said segment, as between said test group and said control group.

23. An isolated polynucleotide segment, comprising a polynucleotide sequence which is selected from the group consisting of:
(a) a sequence comprising SEQ ID NO:5;
(b) a sequence comprising SEQ ID NO:10;
(c) a sequence comprising SEQ ID NO:15;
(d) a sequence comprising SEQ ID NO:20;
(e) a sequence comprising SEQ ID NO:20;
(f) a sequence comprising SEQ ID NO:26;
(g) a sequence which is at least 80% homologous with a sequence of any of (a) to (f);
(h) a sequence which is at least 90% homologous with a sequence of any of (a) to (f):
(i) a sequence which is at least 95% homologous with a sequence of any of (a) to (f):
(j) a sequence which is at least 98% homologous with a sequence of any of (a) to (f):
(k) a sequence which is at least 99% homologous with a sequence of any of (a) to (f). and;
(l) a sequence which hybridizes to any of (a) to (k) under stringent conditions.

24. An isolated polynucleotide segment of claim 23, wherein the isolated polynucleotide segment is genomic DNA.

25. A vector comprising a polynucleotide segment of claim 23 in a suitable vector.

26. A host cell comprising a polynucleotide segment of claim 23 in a host cell which is heterologous to said segment.

27. A process for producing a polypeptide encoded by a gene operably linked to a polynucleotide segment of claim 23 comprising the step of culturing the host cell of claim 26 under conditions sufficient for the production of said polypeptide.

28. An isolated polynucleotide fragment selected from the group consisting of:
(a) a sequence having at least 15 sequential bases of nucleotides of a segment of claim 23;
(b) a sequence having at least 30 sequential bases of nucleotides of a segment of claim 23; and
(c) a sequence having at least 50 sequential bases of nucleotides of a segment of claim 23.

29. A vector comprising a polynucleotide segment of claim 28 contained in a vector which is heterologous to said segment.

30. An isolated polynucleotide segment, comprising a polynucleotide sequence which retains substantially the same biological function or activity as the polynucleotide encoded by a segment of claim 23.

31. A method for identifying a compound which inhibits or promotes the activity of a polynucleotide segment of claim 23, comprising the steps of:
(a) selecting a control animal having said segment and a test animal having said segment;
(b) treating said test animal using a compound; and,
(c) determining the relative quantity of an expression product of an operably linked polynucleotide to said segment, as between said control animal and said test animal.

32. A method of claim 31, wherein said animals are mammals.

33. A method of claim 32, wherein said mammals are rats.

34. A method for identifying a compound which inhibits or promotes the activity of a polynucleotide segment of claim 23, comprising the steps of:
(a) selecting a host cell of claim 26;
(b) cloning said host cell and separating said clones into a test group and a control group;
(c) treating said test group using a compound; and
(d) determining the relative quantity of an expression product of an operably linked polynucleotide to said segment, as between said test group and said control group.

35. A method for identifying a compound which inhibits or promotes the activity of a polynucleotide segment of claim 23, comprising the steps of:
(a) selecting a test group having a host cell of claim 26, a part thereof or an isolated polynucleotide thereof and a control group;
(b) treating said test group using a compound; and
(c) determining the relative quantity of an expression product of an operably linked polynucleotide to said segment, as between said test group and said control group.

36. A composition for treating a disorder involving fatty acid regulated genes, said composition comprising a compound which modulates a segment according to any one of claims 1, 14 or 23 and a pharmaceutically acceptable carrier.

37. A composition as claimed in claim 36 wherein said disorder is a PUFA disorder.

38. A composition as claimed in claim 37, wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

39. A composition as claimed in claim 36, wherein said composition is selected from the group consisting of small organic molecules, peptides, polypeptides, antisense molecules, oligonucleotides, polynucleotides, fatty acids, and functional and chemical derivatives thereof.

40. The use of a composition as claimed in claim 36 for treating a disorder involving fatty acid regulated genes.

41. The use of a composition as claimed in claim 40 for treating a PUFA disorder.

42. The use of claim 41 wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

43. A method for diagnosing the presence of or a predisposition for a disorder involving fatty acid regulated genes in a subject, said method comprising detecting a germline alteration in a segment of claims 1 or 23 in said subject, comprising comparing the germline sequence of a segment of claims 1 or 23 from a tissue sample from said subject with the germline sequence of a wild-type of said segment, wherein an alteration in the germline sequence of said subject indicates the presence of or a predisposition to said disorder.

44. A method for diagnosing the presence of or a predisposition for a disorder as claimed in claim 43 wherein said disorder is a PUFA disorder.

45. A method for diagnosing the presence of or a predisposition for a disorder as claimed in claim 44 wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

46. The method of any one of claims 43 to 45, wherein said comparing is performed by a method selected from the group consisting of immunoblotting, immunocytochemistry, enzyme-linked immunosorbent assay, DNA fingerprinting, in situ hybridization, polymerase chain reaction, reverse transcription polymerase chain reaction, radioimmunoassay, immunoradiometric assay and immunoenzymatic assay.

47. A method for diagnosing the presence of or a predisposition for a disorder involving fatty: regulated genes in a subject, said method comprising comparing the sequence of a polypeptide of claim 14 from a tissue sample from said subject with the sequence of a wild-type of said polypeptide wherein an alteration in the sequence of said subject as compared to said wild-type indicates the presence of or a predisposition to said disorder involving genes altered by fatty acids.

48. A method for diagnosing the presence of or a predisposition for a disorder as claimed in clain 47 wherein said disorder is a PUFA disorder.

49. A method for diagnosing the presence of or a predisposition for a disorder as claim in claim 48 wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

50. The method of any one of claims 47 to 49, wherein said comparing is performed by a metl selected from the group consisting of blotting immunocytochemistry, enzyme-linked immunosorb assay, DNA fingerprinting, radioimmunoassay, immunoradiometric assay, immunoenzymatic assay and polypeptide microarrays.

51. A method for identifying a compound which inhibits or promotes the activity of a polynucleotide, wherein the polynucleotide selected from the group consisting of SCD1, SCD2. SCD, G6PD, GPAT, FAS, COMT, ApoA-1, INSIG1, GLOL, Spot-14 and delta-3, delta-2-enoyl-CoA isomerase, comprising the steps of:
(a) selecting a control animal having said polynucleotide and a test animal having said polynucleotide;
(b) treating said test animal using a compound; and,
(c) determining the relative quantity of an expression product of said polynucleotide, as between said control animal and said test animal.

52. A method of claim 51, wherein said animals are mammals.

53. A method of claim 52, wherein said mammals are rats.

54. A method for identifying a compound which inhibits or promotes the activity of a polynucleotide, wherein the polynucleotide selected from the group consisting of SCD1, SCD2. SCD, G6PD, GPAT, FAS, COMT , ApoA-1, INSIG1, GLOL, Spot-14 and delta-3, delta-2-enoyl-CoA isomerase, comprising the steps of:
(a) selecting, a host cell comprising said polynucleotide wherein such host cell is heterologous to said polynucleotide;
(b) cloning said host cell and separating said clones into a test group and a control group;
(c) treating said test group using a compound; and
(d) determining the relative quantity of an expression product of said polynucleotide, as between said test group and said control group.

55. A method for identifying a compound which inhibits or promotes the activity of a polynucleotide, wherein the polynucleotide selected from the group consisting of SCD1, SCD2, SCD, G6PD, GPAT, FAS, COMT, ApoA-1, INSIG1, GLOL, Spot-14 and delta-3, delta-2-enoyl-CoA isomerase, comprising the steps of:
(a) selecting a test group having a host cell comprising said polynucleotide wherein such host cell is heterologous to said polynucleotide, a part thereof or an isolated polynucleotide thereof, and a control group;
(b) treating said test group using a compound; and
(c) determining the relative quantity or relative activity of a product of said polynucleotide or of the said polynucleotide, as between said test group and said control group.

56. A method for identifying a compound which inhibits or promotes the activity of a polypeptide, wherein the polypeptide selected from the group consisting of SCD1, SCD2, SCD, G6PD, GPAT, FAS, COMT, ApoA-1, INSIG1, GLOL, A1BG, Spot-14 and delta-3, delta-2-enoyl-CoA isomerase, comprising the steps of:
(a) selecting a control animal having said polypeptide and a test animal having said polypeptide;
(b) treating said test animal using a compound;
(c) determining the relative quantity or relative activity of an expression product of said polypeptide or of the said polypeptide, as between said control animal and said test animal.

57. A method of claim 56, wherein said animals are mammals.

58. A method of claim 57, wherein said mammals are rats.

59. A method for identifying which inhibits or promotes the activity of a polypeptide, wherein the polypeptide selected from the group consisting of SCD1, SCD2, SCD, G6PD, GPAT, FAS, COMT, ApoA-1, INSIG1, GLOL, A1BG, Spot-14 and delta-3, delta-2-enoyl-CoA isomerase, comprising the steps of:
(a) selecting a host cell comprising said polypeptide wherein such host cell is heterologous to said polypeptide;
(b) cloning said host cell and separating said clones into a test group and a control group;
(c) treating said test group using a compound; and
(d) determining the relative quantity or relative activity of an expression product of said polypeptide or of the said polypeptide, as between said test group and said control group.

60. A method for identifying a compound which inhibits or promotes the activity of a polypeptide, selected from the group consisting of SCD1, SCD2, SCD, G6PD, GPAT, FAS, COMT, ApoA-1, INSIG₁, GLOL, A1BG, Spot-14 and delta-3, delta-2-enoyl-CoA isomerase, comprising the steps of:
(a) selecting a test group having a host cell comprising said polypeptide wherein such host cell is heterologous to said polypeptide, a part thereof or an isolated polypeptide thereof and a control group;
(b) treating said test group using a compound; and
(c) determining the relative quantity or relative activity of a product of said polypeptide or of the said polypeptide, as between said test group and said control group.

61. A method for identifying a compound which inhibits or promotes the activity of a human polynucleotide, wherein the human polynucleotide is a control region of a gene selected from the group consisting of G6PD, FAS, COMT, ApoA-1, INSIG1, Spot-14 and delta-3, delta-2-enoyl-CoA isomerase, comprising the steps of:
(a) selecting a control animal having said human polynucleotide and a test animal having said human polynucleotide;
(b) treating said test animal using a compound; and,
(c) determining the relative quantity of an expression product of an operably linked polynucleotide to said human polynucleotide, as between said control animal and said test animal.

62. A method of claim 61, wherein said animals are mammals.

63. A method of claim 62, wherein said mammals are rats.

64. A method for identifying a compound which inhibits or promotes the activity of a human polynucleotide, wherein the human polynucleotide is a control region of a gene selected from the group consisting of G6PD, FAS, COMT, ApoA-1, INSIG1, Spot-14 and delta-3, delta-2-enoyl-CoA isomerase, comprising the steps of:
(a) selecting a host cell comprising said human polynucleotide wherein such host cell is heterologous to said polynucleotide;
(b) cloning said host cell and separating said clones into a test group and a control group;
(c) treating said test group using a compound; and
(d) determining the relative quantity of an expression product of an operably linked polynucleotide to said human polynucleotide, as between said test group and said control group.

65. A method for identifying a compound which inhibits or promotes the activity of a human polynucleotide, wherein the human polynucleotide is a control region of a gene selected from the group consisting of G6PD, FAS, COMT, ApoA-1, INSIG1, Spot-14 and delta-3, delta-2-enoyl-CoA isomerase, comprising the steps of:
(a) selecting a test group having a host cell comprising said human polynucleotide wherein such host cell is heterologous to said polynucleotide, a part thereof or an isolated polynucleotide thereof and a control group;
(b) treating said test group using a compound; and
(c) determining the relative quantity of an expression product of an operably linked polynucleotide to said human polynucleotide, as between said test group and said control group.

66. A composition for treating a disorder involving fatty acid regulated genes, said composition comprising a compound which modulates a polynucleotide from the coding sequence selected from the group consisting of SCD1, SCD2, SCD, G6PD, GPAT, FAS, COMT, ApoA-1, INSIG1, GLOL, Spot-14 and delta-3, delta-2-enoyl-CoA isomerase and a pharmaceutically acceptable carrier.

67. A composition for treating a fatty acid disorder comprising a compound which modulates a polypeptide selected from the group consisting of SCD1, SCD2, SCD, G6PD, GPAT, FAS. COMT, ApoA-1, INSIG1, GLOL, A1BG, Spot-14 and delta-3, delta-2-enoyl-CoA isomerase and a pharmaceutically acceptable carrier.

68. A composition for treating a fatty acid disorder comprising a compound which modulates a control region selected from the group consisting of G6PD, FAS, COMT, ApoA-1, INSIG1, Spot-14 and delta-3, delta-2-enoyl-CoA isomerase and a pharmaceutically acceptable carrier.

69. A composition as claimed in any one of claims 66 to 68 wherein said disorder is a PUFA disorder.

70. A composition as claimed in claim 69, wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

71. A composition as claimed in any one of claims 66 to 68, wherein said compound is selected from the group consisting of small organic molecules, peptides, polypeptides, antisense molecules, oligonucleotides, polynucleotides, fatty acids, and functional and chemical derivatives thereof.

72. The use of a composition as claimed any one of claims 66 to 68 for treating a fatty acid disorder.

73. The use of a composition as claimed in claim 72 for treating a PUFA disorder.

74. The use of claim 73 wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

75. A method for diagnosing the presence of or a predisposition for a disorder involving fatty acid regulated genes in a subject, said method comprising detecting a germline alteration in a polynucleotide representing the coding sequence selected from the group consisting of SCD1, SCD2, SCD, G6PD, GPAT, FAS, COMT, ApoA-1, INSIG1, GLOL, Spot-14 and delta-3, delta-2-enoyl-CoA isomerase in said subject, comprising comparing the germline sequence of said polynucleotide from a tissue sample from said subject with the germline sequence of a wild-type of said polynucleotide, wherein an alteration in the germline sequence of said subject indicates the presence of or a predisposition to said disorder.

76. A method for diagnosing the presence of or a predisposition for a disorder involving fatty acid regulated genes in a subject, said method comprising detecting a germline alteration in a human polynucleotide representing the control region selected from the group consisting of G6PD, FAS, COMT, ApoA-1, INSIG1, Spot-14 and delta-3, delta-2-enoyl-CoA isomerase in said subject, comprising comparing the germline sequence of said human polynucleotide from a tissue sample from said subject with the germline sequence of a wild-type of said human polynucleotide, wherein an alteration in the germline sequence of said subject indicates the presence of or a predisposition to said disorder.

77. A method for diagnosing the presence of or a predisposition for a disorder as claimed in claims 75 or 76 wherein said disorder is a PUFA disorder.

78. A method for diagnosing the presence of or a predisposition for a disorder as claimed in claim 77 wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

79. The method of any one of claims 75 to 78, wherein said comparing is performed by a method selected from the group consisting of immunoblotting, immunocytochemistry, enzyme-linked immunosorbent assay, DNA fingerprinting, in situ hybridization, polymerase chain reaction, reverse transcription polymerase chain reaction, radioimmunoassay, immunoradiometric assay and immunoenzymatic assay.

80. A method for diagnosing the presence of or a predisposition for a disorder involving fatty acid regulated genes in a subject, said method comprising comparing the sequence of a polypeptide selected from the group consisting of SCD1, SCD2, SCD, G6PD, GPAT, FAS, COMT, ApoA-1, INSIG1, GLOL, A1BG, Spot-14 and delta-3, delta-2-enoyl-CoA isomerase from a tissue sample from said subject with the sequence of a wild-type of said polypeptide, wherein an alteration in the sequence of said subject as compared to said wild-type indicates the presence of or a predisposition to said disorder involving genes altered by fatty acids.

81. A method for diagnosing the presence of or a predisposition for a disorder as claimed in claim 80 wherein said disorder is a PUFA disorder.

82. A method for diagnosing the presence of or a predisposition for a disorder as claimed in claim 81 wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

83. The method of any one of claims 80 to 82, wherein said comparing is performed by a method selected from the group consisting of immunoblotting, immunocytochemistry, enzyme-linked immunosorbent assay, DNA fingerprinting, radioimmunoassay, immunoradiometric assay, immunoenzymatic assay and polypeptide microarrays.

84. A method for identifying a compound which inhibits or promotes a disorder involving fatty acid regulated genes, said method comprising the steps of:
(a) selecting a control animal and a test animal both having a gene selected from the group consisting of SCD1, SCD2, SCD, G6PD, GPAT, FAS, COMT, ApOA-1, INSIG1, FTF1, GLOL, A1GB, Spot-14, delta-3, delta-2-enoyl-CoA isomerase, and METP or a control region sequence thereof;
(b) treating said test animal using a compound; and,
(c) determining the relative quantity of an expression product of said gene, as between said control animal and said test animal.

85. A method of claim 84, wherein said animals are mammals.

86. A method of claim 85, wherein said mammals are rats.

87. A method of any one of claims 84 to 86, wherein said disorder is a PUFA disorder.

88. A method of claim 87, wherein said disorder is selected from a group consisting of cancer, cystic fibrosis, pre-menstrual syndrome, diabetes and diabetic complications.

89. A method for identifying a compound which inhibits or promotes a disorder involving fatty acid regulated genes, said method comprising the steps of:
(a) selecting a host cell containing a gene selected from the group consisting of SCD1, SCD2, SCD, G6PD, GPAT, FAS, COMT, ApoA-1, INSIG1, FTF1, GLOL, A1GB, Spot14, delta-3, delta-2-enoyl-CoA isomerase, and METP or a control region sequence thereof;
(b) cloning said host cell and separating said clones into a test group and a control group;
(c) treating said test group using a compound; and,
(d) determining the relative quantity of an expression product of said gene, as between said test group and said control group.

90. A method of claim 89, wherein said disorder is a PUFA disorder.

91. A method of claim 90, wherein said disorder is selected from a group consisting of eczema, cardiovascular disorders, inflammation, body weight disorders, psychiatric disorders, cancer, cystic fibrosis, pre-menstrual syndrome, diabetes and diabetic complications.

92. A method for detecting the presence of or the predisposition for a disorder involving fatty acid regulated genes, said method comprising determining the level of expression of an expression product of a gene selected from a polynucleotide segment of claim 1 in a subject relative to a predetermined control level of expression, wherein a modified expression of said expression product as compared to said control is indicative of the presence of or the predisposition for a disorder involving genes altered by fatty acids.

93. A method for detecting the presence of or the predisposition for a disorder involving genes altered by fatty acids, said method comprising determining the level of expression of an expression product of a gene selected from the group consisting of SCD 1, SCD2, SCD, G6PD, GPAT, FAS, COMT, ApoA-1, INSIG1, Spot-14, delta-3, delta-2-enoyl-CoA isomerase, FTF1, GLOL, A1BG and METP, in a subject relative to a predetermined control level of expression, wherein a modified expression of said expression product as compared to said control is indicative of the presence of or the predisposition for a disorder involving genes altered by fatty acids.

94. A method of claims 92 or 93, wherein said disorder is a PUFA disorder.

95. A method of claim 94, wherein said disorder is selected from a group consisting of eczema, cardiovascular disorders, inflammation, body weight disorders, psychiatric disorders, cancer, cystic fibrosis, pre-menstrual syndrome, diabetes and diabetic complications.

96. A method of any one of claims 92 to 95 wherein said method is selected from the group consisting of immunoblotting, immunocytochemistry, enzyme-linked immunosorbent assay, in situ hybridization, reverse transcription polymerase chain reaction, radioimmunoassay, immunoradiometric assay, immunoenzymatic assay and polynucleotide and polypeptide microarrays.

97. An antibody immunoreactive with a polypeptide of claim 14 or an immunogenic portion thereof.

98. An antibody immunoreactive with a polypeptide selected from the group consisting of FTF1, GLOL and METP or an immunogenic portion thereof.

99. A method for screening a medium for a polypeptide of claim 14 or selected from the group consisting of FTF1, GLOL and METP, comprising:
(a) labelling an antibody of claims 97 or 98 with a marker molecule to form a conjugate;
(b) exposing said conjugate to said medium; and
(c) determining whether there is binding between said conjugate and a biomolecule in said medium, wherein said binding indicates the presence of said polypeptide.

100. A method for screening a medium for a polypeptide of claim 14 or selected from the group consisting of FTF1, GLOL and METP, comprising;
(a) exposing an antibody of claims 97 or 98 to said medium;
(b) exposing said antibody to a marker molecule; and
(c) determing whether there is binding between said marker molecule and a biomolecule in said medium, wherein said binding indicates the presence of said polypeptide.

101. A composition as claimed in claim 36, wherein said compound comprises an antibody of claims 97 or 98.

102. A composition as claimed in any one of claims 66 to 68, wherein said composition is selected from the group consisting of antibodies against SCD1, SCD2, SCD, G6PD, GPAT, FAS, COMT, ApoA-1, INSIG1, A1GB, Spot-14, and delta-3, delta-2-enoyl-CoA isomerase.

103. The use of a composition as claimed in claims 101 or 102 for treating a disorder involving genes altered by fatty acids.

104. The use as claimed in claim 103, wherein said disorder is a PUFA disorder.

105. The use of claim 104 wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

106. A method for identifying genes or proteins regulated by fat, comprising:
(a) selecting a species of animals and separating them into a test group and a control group;
(b) feeding said test group and said control group a fat free diet for a period of time;
(c) subsequently providing said test group enterally or parenterally with highly purified polyunsaturated fatty acids for a second period of time;
(d) subsequently removing tissues from said control group and said test group;
(e) comparing RNA from said tissues of said control group with tissues from said test group and selecting RNA which is expressed at a different level as between said test group and said control group; and
(f) determining the genes or proteins associated with said selected RNA.

107. A method as claimed in claim 106, wherein said animals are rats.

108. A method as claimed in claim 106 wherein said period of time is at least 2 weeks.

109. A method as claimed in claim 106 wherein said second period of time is at least 2 weeks.

110. A method as claimed in claim 106 wherein said comparing is by differential display and Northern blotting.

111. A method for identifying a compound which inhibits or promotes the activity of two or more human polynucleotides, wherein the human polynucleotides are control regions of genes selected from the group consisting of METP, GLOL, FTF1, A1BG, SCD, GPAT, G6PD, FAS, COMT, ApoA-1, INSIG1, Spot-14 and delta-3, delta-2-enoyl-CoA isomerase, said method comprising the steps of:
(a) selecting two or more host cells comprising said human polynucleotides wherein such host cells are heterologous to said polynucleotides;
(b) cloning said host cells and separating said clones into a test group and a control group;
(c) treating said test group using a compound; and
(d) determining the relative quantities of expression products of operably-linked polynucleotides to said human polynucleotides, as between said test group and said control group.

112. A method for identifying a compound which inhibits or promotes the activity of two or more human polynucleotides, wherein the human polynucleotides are control regions of genes selected from the group consisting of METP, GLOL, FTF1, A1BG, SCD, GPAT, G6PD, FAS, COMT, ApoA-1, INSIG1, Spot-14 and delta-3, delta-2-enoyl-CoA isomerase, comprising the steps of:
(a) selecting a test group having two or more host cells containing said human polynucleotides wherein such host cells are heterologous to said polynucleotides, parts thereof or isolated polynucleotides thereof and a control group;
(b) treating said test group using a compound; and
(c) determining the relative quantities of expression products of operably linked polynucleotides to said human polynucleotides, as between said test group and said control group.

113. A composition for treating a disorder involving fatty acid regulated genes comprising a compound which modulates two or more human polynucleotide control regions of genes selected from the group consisting of of METP, GLOL, FTF1, A1BG, SCD, GPAT, G6PD, FAS, COMT, ApoA-1, INSIG1, Spot-14 and delta-3, delta-2-enoyl-CoA isomerase and a pharmaceutically acceptable carrier.

114. A composition as claimed in claim 113 wherein said disorder is a PUFA disorder.

115. A composition as claimed in claim 114, wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

116. A composition as claimed in any one of claims 113 to 115, wherein said compound is selected from the group consisting of small organic molecules, peptides, polypeptides, antisense molecules, oligonucleotides, polynucleotides, fatty acids, and functional and chemical derivatives thereof.

117. The use of a composition as claimed in claims 113 for treating a disorder involving genes altered by fatty acids.

118. The use of a composition as claimed in claim 117 for treating a PUFA disorder.

119. The use of claim 118 wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

120. A method for detecting the presence of or the predisposition for a disorder involving genes altered by fatty acids, said method comprising determining the level of expression of two or more expression products of genes selected from the group consisting of human SCD, G6PD, GPAT, FAS, COMT, ApoA-1, INSIG1, Spot-14, delta-3, delta-2-enoyl-CoA isomerase, FTF1, GLOL, A1BG and METP, in a subject relative to a predetermined control level of expression, wherein any modified expression of said expression products as compared to said control indicates the presence of or the predisposition for a disorder involving genes altered by fatty acids.

121. A method of claim 120, wherein said disorder is a PUFA disorder.

122. A method of claim 121, wherein said disorder is selected from a group consisting of eczema, cardiovascular disorders, inflammation, body weight disorders, psychiatric disorders, cancer, cystic fibrosis, pre-menstrual syndrome, diabetes and diabetic complications.

123. A method of any one of claims 120 to 122 wherein said method is selected from the group consisting of immunoblotting, immunocytochemistry, enzyme-linked immunosorbent assay, in situ hybridization, reverse transcription polymerase chain reaction, radioimmunoassay, immunoradiometric assay, immunoenzymatic assay and polynucleotide and polypeptide microarrays.

124. A compound identified by the methods of any one of claims 9 to 13, 18 to 22, 31 to 35, 51 to 65, 84 to 91 or 111 to 112.

125. The use of a compound as claimed in claim 124 for treating a disorder involving fatty acid regulated genes.

126. The use of a compound as claimed in claim 125 wherein said disorder is a PUFA disorder.

127. The use of claim 126 wherein said disorder is selected from the group consisting of eczema, cardiovascular disorders, inflammation, body weight disorders, psychiatric disorders, cancer, cystic fibrosis, pre-menstrual syndrome, diabetes and diabetic complications.

128. The composition as claimed in any one of claims 38, 70 and 115, wherein the cardiovascular disorder is selected from the group consisting of hypertriglyceridemia, dyslipidemia, atherosclerosis, coronary artery disease, cerebrovascular disease and peripheral vascular disease.

129. The use as claimed in any one of claims 42, 74, 119, and 127, wherein the cardiovascular disorder is selected from the group consisting of hypertriglyceridemia, dyslipidemia, atherosclerosis, coronary artery disease, cerebrovascular disease and peripheral vascular disease.

130. The method as claimed in any one of claims 45, 49, 78, 81, 91, 95, 105 and 122, wherein the cardiovascular disorder is selected from the group consisting of hypertriglyceridemia, dyslipidemia, atherosclerosis, coronary artery disease, cerebrovascular disease and peripheral vascular disease.

131. The composition as claimed in any one of claims 38, 70 and 115, wherein the inflammation is selected from the group consisting of sinusitis, asthma, pancreatitis, osteoarthritis, rheumatoid arthritis and acne.

132. The use as claimed in any one of claims 42, 74, 119, and 127, wherein the inflammation is selected from the group consisting of sinusitis, asthma, pancreatitis, osteoarthritis, rheumatoid arthritis and acne.

133. The method as claimed in any one of claims 45, 49, 78, 81, 91, 95, 105 and 122, wherein the inflammation is selected from the group consisting of sinusitis, asthma, pancreatitis, osteoarthritis, rheumatoid arthritis and acne.

134. The composition as claimed in any one of claims 38, 70 and 115, wherein the body weight disorder is selected from the group consisting of obesity, cachexia and anorexia.

135. The use as claimed in any one of claims 42, 74, 119, and 127, wherein the body weight disorder is selected from the group consisting of obesity, cachexia and anorexia.

136. The method as claimed in any one of claims 45, 49, 78, 81, 91, 95, 105 and 122, wherein the body weight disorder is selected from the group consisting of obesity, cachexia and anorexia.
